(19) United States
(12) Patent Application Publication
(10) Pub. No.: US 2022/0155281 A1
(43) Pub. Date: May 19, 2022

(54) PROCESS CONTROL IN CELL BASED ASSAYS

(71) Applicant: Recursion Pharmaceuticals, Inc., Salt Lake City, UT (US)

(72) Inventors: Mason VICTORS, Sandy, UT (US); August ALLEN, Salt Lake City, UT (US); Berton Allen EARNSHAW, Cedar Hills, UT (US); Blake BORGESON, San Francisco, CA (US); Cedric ST-JEAN-LEBLANC, Montreal (CA)

(73) Assignee: Recursion Pharmaceuticals, Inc., Salt Lake City, UT (US)

(21) Appl. No.: 17/439,450

(22) PCT Filed: Mar. 11, 2020
(86) PCT No.: PCT/US20/22048

§ 371 Date: Sep. 15, 2021

Related U.S. Application Data
(60) Provisional application No. 62/819,375, filed on Mar. 15, 2019.

(51) Int. Cl. G01N 33/50; C12N 15/113; G01N 35/00; G16B 5/20; G16B 40/00

(52) U.S. Cl. CPC G01N 33/5008; C12N 15/113; G01N 35/00069; C12N 2310/14; G16B 40/00; G01N 2500/10; G01N 2035/00158; G16B 5/20

(57) ABSTRACT

Evaluating an effect of one or more perturbations on cells of a first cell type is described. One method includes obtaining a screen definition for a screen, where the screen includes a cell-based assay that is run on a temporarily contiguous basis using a plurality of multi-well plates. The method includes obtaining control vectors including measurements of corresponding features of cells in control wells. The method includes obtaining test vectors including measurements of corresponding features of cells in test wells. The method includes forming a variability model based on a variance across the of control vectors, and embedding test vectors onto the variability model, thereby obtaining a set of variability model values. The method includes using the set of variability model values to resolve an effect of at least one data perturbation in the plurality of data perturbations on the first cell type.

### CROSS REFERENCE TO RELATED APPLICATIONS

This application claims priority to and benefit of co-pending U.S. Provisional Patent Application No. 62/819,375 filed on Mar. 15, 2019 entitled “SYSTEMS AND METHODS FOR PROCESS CONTROL IN CELL BASED ASSAYS” by Mason Victors et al., having Attorney Docket No. 118623-5005-PR, and assigned to the assignee of the present application, the disclosure of which is hereby incorporated herein by reference in its entirety.

### BACKGROUND ART

There are approximately 6,000 rare diseases affecting an estimated 25 million people in the United States. Rare diseases disproportionately affect children, and many children with rare genetic diseases do not live to see their 5th birthday. Therapeutic development for these diseases has been slow, and less than 5% of rare diseases have an FDA-approved. This is due in part to the conventional requirement for a substantial understanding of the disease and the corresponding physiology prior to the design and implementation of a drug discovery strategy. Swinney and Xia, 2014, Future Med. Chem. 6(9):987-1002. However, often times, such understanding of rare diseases and their corresponding physiology does not exist, hindering the development of assays required for drug discovery.

High throughput screening (HTS) is a process used in pharmaceutical drug discovery to test large compound libraries containing thousands to millions of compounds for various biological effects. HTS typically uses robotics, such as liquid handlers and automated imaging devices, to conduct tens of thousands to tens of millions of assays, e.g., biochemical, genetic, and/or phenotypical, on the large compound libraries in multi-well plates, e.g., 96-well, 384-well, 1536-well, or 3456-well plates. In this fashion, lead-compounds that provide a desired biochemical, genetic, or phenotypic effect can be quickly identified from the large compound libraries, for further testing and development towards the goal of discovering a new pharmaceutical agent for disease treatment. For a review of basic HTS methodologies see, for example, Wildey et al., 2017, “Chapter Five—High-Throughput Screening,” Annual Reports in Medicinal Chemistry, Academic Press, 50:149-95, which is hereby incorporated by reference.

### BRIEF DESCRIPTION OF DRAWINGS

The accompanying drawings, which are incorporated in and form a part of the Description of Embodiments, illustrate various embodiments of the subject matter and, together with the Description of Embodiments, serve to explain principles of the subject matter discussed below. Unless specifically noted, the drawings referred to in this Brief Description of Drawings should be understood as not being drawn to scale. Herein, like reference numerals refer to corresponding parts throughout the several views of the drawings.

FIG. 1 illustrates an exemplary workflow for evaluating an effect of one or more perturbations on cells, in accordance with various embodiments of the present disclosure.

FIGS. 2A, 2B, and 2C collectively illustrate a device for evaluating an effect of one or more perturbations on cells, in accordance with various embodiments of the present disclosure.

FIG. 3 illustrates an example process for obtaining feature data for an effect of one or more perturbations on cells, in accordance with various embodiments of the present disclosure.

FIGS. 4A and 4B collectively illustrate an example process for training a variability model for use in evaluating an effect of one or more perturbations on cells, in accordance with various embodiments of the present disclosure.

FIGS. 5A and 5B collectively illustrate an example process for evaluating an effect of one or more perturbations on cells using a trained variability model, in accordance with various embodiments of the present disclosure.

FIGS. 6A, 6B, and 6C collectively illustrate an example process for training principal components for use in evaluating an effect of one or more perturbations on cells, in accordance with various embodiments of the present disclosure.

FIGS. 7A and 7B collectively illustrate an example process for evaluating an effect of one or more perturbations on cells using trained principal components, in accordance with various embodiments of the present disclosure.

FIG. 8 depicts an example method for evaluating an effect of one or more perturbations on cells of a first cell type, in accordance with various embodiments.

FIG. 9 shows 6-channel faux-colored composite image of HUVEC cells and individual channels: nuclei (blue), endoplasmic reticuli (green), actin (red), nucleoli and cytoplasmic RNA (cyan), mitochondria (magenta), and Golgi (yellow). The similarity in content between some channels is due in part to the spectral overlap between the fluorescent stains used in those channels.

FIG. 10 shows images of four different siRNA phenotypes. These images are from the same plate in a HUVEC experiment.

FIG. 11 show Images of the same siRNA in four cell types: HUVEC, RPE, HepG2, and U2OS.

FIG. 12 shows images of two different siRNA (rows) in HUVEC cells across four experimental batches (columns). Notice the visual similarity of images from the same batch.

### DESCRIPTION OF EMBODIMENTS

### Overview

Rare diseases represent an urgent area of great unmet medical need. This is due, in part, because conventional methods for screening compounds for drug identification rely on the development of a robust model assay for the disease. Because the sales potential for drugs treating rare diseases is low, there is much less incentive to spend the considerable time and resources necessary to develop such a robust model assay. Advantageously, the present disclosure addresses this need by disclosing drug discovery screening platforms that are quickly adaptable for use in screening compound libraries for any disease state, regardless of whether a model assay for the disease has been developed. The screening platform described herein leverages high-dimensional structural phenotypes across many different cellular perturbations in massively parallel high-throughput drug screens.

For example, in some embodiments, the methods, systems, and software described herein improve upon HTS by using control systems that facilitate comparison of large experiments run over an extended period of time. For example, in some embodiments, a control system creates a mathematical space in which variation within multi-dimensional phenotypic data is represented in a mathematical space defined by a series of control experiments. This decouples the significance of individual phenotypes from the test assays themselves, such that the mathematical space can be recreated later without having to re-run all of the test assays again. In this fashion, comparable statistical tests can be performed across different experiments.

Because HTS is dependent upon the development of a biological assay to screen against, HTS cannot conventionally be implemented for rare diseases for which a substantial understanding of the disease and the corresponding physiology does not exist. What is needed in the art and what is described herein are improved systems and methods for screening compound libraries to identify candidate therapies, e.g., particularly for rare diseases where a substantial understanding of the disease and the corresponding physiology does not yet exist. The present disclosure addresses, among others, the need for systems and methods that facilitate intelligent screening of chemical compound libraries without a subsequence understanding of the disease and the corresponding physiology. Further, the systems and methods described herein facilitate identification of compounds that rescue disease phenotype.

The methods and systems disclosed herein leverage automated biology and artificial intelligence. In some embodiments, the use of microscopy to measure hundreds of sub-cellular structural changes caused by pathogenic perturbations facilitates discovery of data-rich “marker-less” high-dimensional phenotypes in vitro across many individual disease models. High-throughput drug screens on these phenotypes uncovers promising drug candidates that rescue disease signatures. This unique approach allows rapid modeling and screening for potential treatments for hundreds of traditionally refractory diseases, making it ideally suited to tackle the urgent unmet medical need of patients with rare diseases.

In one aspect, the disclosure provides methods, systems, and computable readable media for evaluating an effect of one or more perturbations on cells of a first cell type. The methods include obtaining a screen definition for a screen, where the screen includes a cell-based assay, e.g., that is run on a temporarily contiguous basis, using a plurality of multi-well plates. the screen definition identifies a first plurality of control wells and a plurality of data wells in the plurality of multi-well plates. Each respective control well in the first plurality of control wells is labeled with a control perturbation label corresponding to a control perturbation in a first plurality of control perturbations that is independently included in the respective control well. Each respective data well in the plurality of data wells is labeled with a data perturbation label corresponding to a data perturbation in a plurality of data perturbations that is independently included in the respective data well. An aliquot of cells of the first cell type is included in each control well in the first plurality of control wells and in each data well in the plurality of data wells. The method includes obtaining, for each respective control well in the first plurality of control wells, a corresponding control vector including a plurality of elements, each respective element in the plurality of elements of the corresponding control vector including a measurement of a corresponding feature, in a plurality of features, of the aliquot of cells of the first cell type in the respective control well, thereby obtaining a first plurality of control vectors. The method includes obtaining, for each respective data well in the plurality of data wells, a corresponding data vector including the plurality of elements, each respective element in the plurality of elements of the corresponding data vector including a measurement of a corresponding feature, in the plurality of features, of the aliquot of cells of the first cell type in the respective data well, thereby obtaining a plurality of data vectors. The method includes forming a variability model based, at least in part, on all or a portion of a variance across the first plurality of control vectors, and embedding each data vector in the plurality of data vectors onto the variability model, thereby obtaining a set of variability model values for each data vector in the plurality of data vectors. Advantageously, the set of variability model values and the corresponding data perturbation label of each data well in the plurality of data wells can be used to resolve an effect of at least one data perturbation in the plurality of data perturbations on the first cell type.

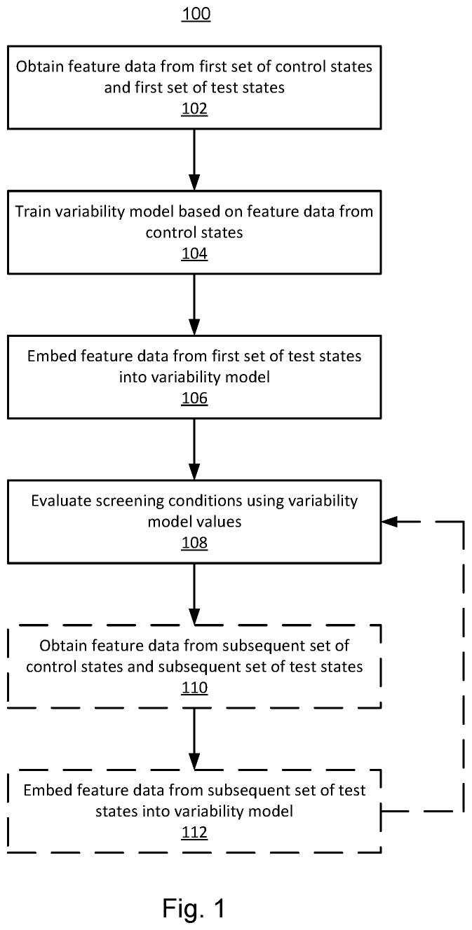

For example, as described below with reference to FIGS. 1 and 4-7, feature measurements from a first set of control experiments, which are performed each time a large phenotypic screen of a particular cell type is run, are used to define a mathematical space that accounts for variability within the control experiments. Test data is then embedded into this mathematical space, decupling individual phenotypic measurements from the overall variance of a particular perturbation, e.g., on cells. In some embodiments, a second set of control experiments, which are performed with each multi-well plate used in each instance of a large phenotypic screen.

### Definitions

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It will also be understood that, although the terms first, second, etc. may be used herein to describe various elements, these elements should not be limited by these terms. These terms are only used to distinguish one element from another. For example, a first control perturbation could be termed a second control perturbation, and, similarly, a second control perturbation could be termed a first control perturbation, without departing from the scope of the present disclosure. The first control perturbation and the second control perturbation are both control perturbations, but they are not the same control perturbation. Furthermore, the terms “subject,” “user,” and “patient” are used interchangeably herein.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “cell context” or “cellular context” refers to an experimental condition including an aliquot of cells of one or more cell types and a chemical environment, a culture medium and optionally a data perturbation, exclusive of a query perturbation, e.g., that does not include a compound of treatment being screened. That is, control states and test states constitute cell contexts, while query perturbation states constitute cell contexts that are exposed to a query perturbation. In some embodiments, the aliquot of cells is of a single cell type.

As used herein, a “perturbation” is an environmental factor that potentially changes a cell context in a measurable way as exhibited by a measureable change in at least one phenotype of the cell. It will be appreciated that not all perturbations in fact cause a measurable change in cell context and the present disclosure is designed to ascertain whether perturbations do, in fact, cause such changes and, in some embodiments, to quantify such changes caused by them. In some embodiments, a perturbation is a chemical composition. In some embodiments, a perturbation causes a cellular phenotype representative of a diseased cell phenotype. In some embodiments, a perturbation is compound that is exposed to, and acts upon, an aliquot of cells, e.g., an siRNA that knocks-down expression of a gene in the cell or a compound that perturbs a cellular process (e.g., inhibits a cellular signaling pathway, inhibits a metabolic pathway, inhibits a cellular checkpoint, etc.). In some embodiments, a perturbation is physical change to the cell context, e.g., a temperature change and/or a change in the surrounding chemical environment (e.g., a change in the nutrient composition of a cell culture medium in which a cell context is growing).

As used herein, a “control perturbation” refers to a perturbation used in an assay condition from which measured feature values will be used to manipulate feature values measured from assay conditions that includes a data perturbation, e.g., through normalization, standardization, or establishment of a phenotypic variation model. In some instances, an assay condition may include both a control perturbation and a compound whose therapeutic effects are being screened. Thus, in some embodiments, an assay condition including a control perturbation is used to both manipulate feature values measured from an assay condition that includes a data perturbation and serve to provide screening data used to evaluate the therapeutic effect of a compound.

As used herein, a “data perturbation” refers to a perturbation used in an assay condition from which measured feature values are not used to manipulate feature values measured from assay conditions employing other data perturbations.

As used herein, the term “control state” refers to an assay condition that includes a cell context that is perturbed by a control perturbation. In some embodiments, a control state also includes a compound whose therapeutic effects are being screened.

As used herein, the term “test state” refers to an assay condition that includes a cell context that is perturbed by a data perturbation. In some embodiments, a test state also includes a compound whose therapeutic effects are being screened.

### Compound Screening

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

Referring to FIG. 1, the present disclosure provides a method 100 for evaluating an effect of one or more perturbations and/or therapeutic candidate compounds on cells. The method includes obtaining (102) feature data from a first set of control states and a first set of test states, e.g., which may or may not also include a therapeutic candidate compound. Each control state in the set of control states and each test state in the set of test states includes a common cellular context. The method then includes training (104) a variability model based on feature data from first set of control states. As described below, in some embodiments, the variability model is based on only a subset of all features measured from the control states. Likewise, in some embodiments, the feature values used to train the variability model are normalized, standardized, and/or centered based on feature measurements from a separate set of control states (e.g., that is specific to the multi-well plate from which the control state was located). The method then includes embedding (106) feature data from the first set of test states into the variability model trained on the feature data from the first set of control states. In some embodiments, as described below, the feature values embedded into the variability model are normalized, standardized, and/or centered based on feature measurements from a separate set of control states (e.g., that is specific to the multi-well plate from which the test state was located). The method then includes evaluating (108, 5000) one or more screening conditions (e.g., the effect of a perturbation and/or candidate therapeutic compound on a cellular context) within the mathematical space defined by the trained variability model.

In some embodiment, the method also includes obtaining (110) feature data from a subsequent set of the same control states used in step 102 and a subsequent set of different test states than used in step 102 (or any previously measured test states). The method then includes embedding (112) feature data from the subsequent set of test states into the variability model trained on the feature data from the first set of control states. In some embodiments, as described below, the feature values embedded into the variability model are normalized, standardized, and/or centered based on feature measurements from a separate set of control states (e.g., that is specific to the multi-well plate from which the test state was located). The method then includes evaluating (108) one or more screening conditions (e.g., the effect of a perturbation and/or candidate therapeutic compound on a cellular context) within the mathematical space defined by the trained variability model. Multiple iterations of subsequent screening steps 110 and 112 can be performed.

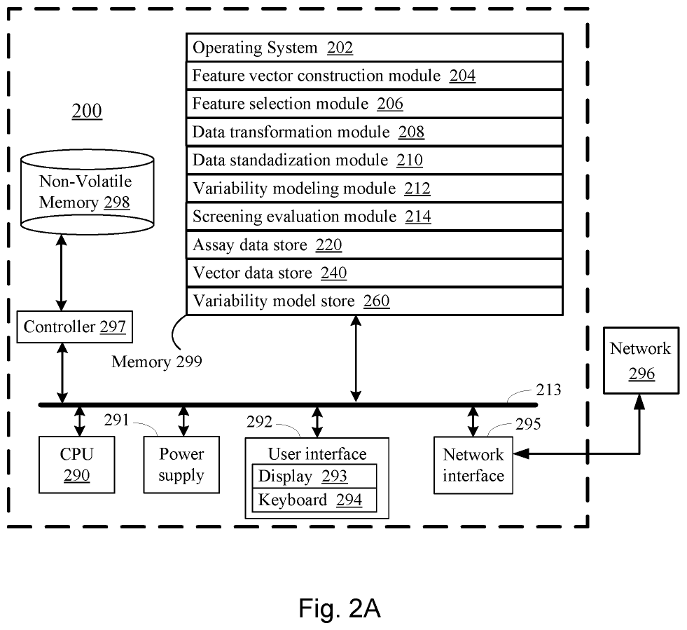

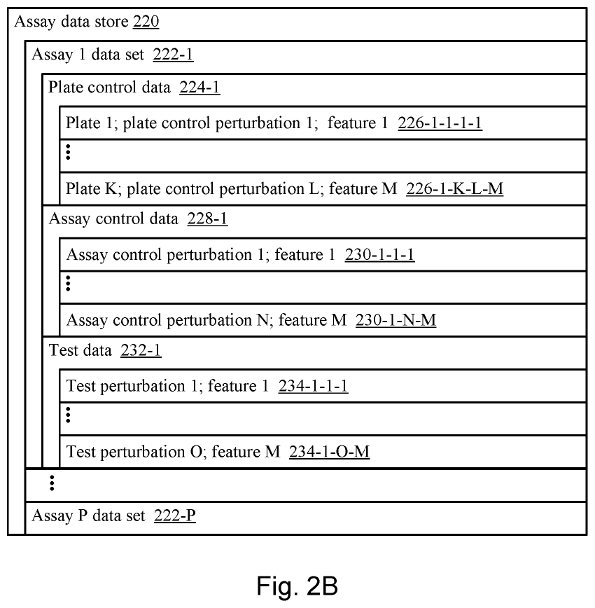

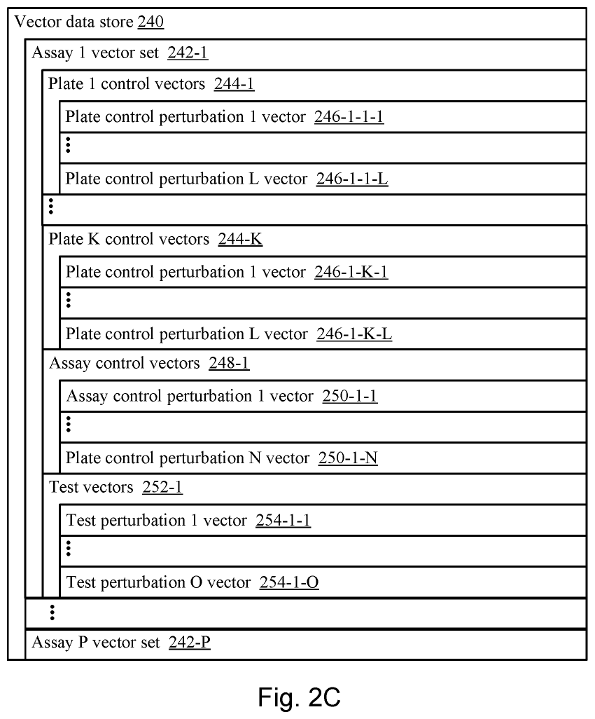

A detailed description of a system 200 for evaluating an effect of one or more perturbations and/or therapeutic candidate compounds on cells is described in conjunction with FIGS. 2A, 2B, and 2C. As such, FIGS. 2A, 2B, and 2C collectively illustrate the topology of a system, in accordance with an embodiment of the present disclosure.

Referring to FIG. 2A, in typical embodiments, system 200 comprises one or more computers. For purposes of illustration in FIG. 2A, system 200 is represented as a single computer that includes all of the functionality for evaluating an effect of one or more perturbations and/or therapeutic candidate compounds on cells. However, the disclosure is not so limited. In some embodiments, the functionality for evaluating an effect of one or more perturbations and/or therapeutic candidate compounds on cells is spread across any number of networked computers and/or resides on each of several networked computers and/or is hosted on one or more virtual machines at a remote location accessible across the communications network 296. One of skill in the art will appreciate that any of a wide array of different computer topologies are used for the application and all such topologies are within the scope of the present disclosure.

With the foregoing in mind, an example system 200 for evaluating an effect of one or more perturbations and/or therapeutic candidate compounds on a cell includes one or more processing units (CPU's) 290, a network or other communications interface 295, a memory 299 (e.g., random access memory), one or more magnetic disk storage and/or persistent devices 298 optionally accessed by one or more controllers 297, one or more communication busses 213 for interconnecting the aforementioned components, a user interface 292, the user interface 292 including a display 293 and input 294 (e.g., keyboard, keypad, touch screen), and a power supply 291 for powering the aforementioned components. In some embodiments, data in memory 299 is seamlessly shared with non-volatile memory 298 using known computing techniques such as caching. In some embodiments, memory 299 and/or memory 298 includes mass storage that is remotely located with respect to the central processing unit(s) 290. In other words, some data stored in memory 299 and/or memory 298 may in fact be hosted on computers that are external to the system 200 but that can be electronically accessed by the system 200 over an Internet, intranet, or other form of network or electronic cable (illustrated as 296 in FIG. 2) using network interface 295.

In some embodiments, the memory 299 of the system 200 for evaluating an effect of one or more perturbations and/or therapeutic candidate compounds on a cell:
an operating system 202 that includes procedures for handling various basic system services;
a feature vector construction module 204, e.g., for constructing plate control vectors 246, assay control vectors 250 and test vectors 254 from measured feature values (226; 230; 234);
a feature selection module 206, e.g., for removing features that provide less than a threshold amount of unique values across a set of assay states;
a data transformation module 208, e.g., for transforming individual feature measurement values by a predetermined function;
a data standardization module 210, e.g., for standardizing, normalizing, and/or centering a set of values (e.g., feature values, transformed feature values, or variability model values);
a variability modeling module 212, e.g., for training variability models on feature measurements of control states and embedding feature measurements of test states into the trained variability model;
a screening evaluation module 214, e.g., for evaluating the effects of a perturbation and/or candidate therapeutic compound on a cell context;
a feature measurement database 220, e.g., for storing assay data sets 222 that include one or more of plate control data 224 (e.g., plate control features measurements 226), assay control data 228 (e.g., assay control features measurements 230), and test data 232 (e.g., test features measurements 234);
a vector database 240, e.g., for storing assay vectors set 242 that include one or more of plate control vectors 244 (e.g., perturbation vectors 246), assay control vectors 248 (e.g., perturbation vectors 250), and test vectors 252 (e.g., perturbation vectors 254); and
a variability model database 260, e.g., for storing variability model value sets (543; 547; 743; 747) constructed by variability modeling module 212.

In some embodiments, modules 204, 206, 208, 210, 212, and/or 214 are accessible within any browser (phone, tablet, laptop/desktop). In some embodiments modules 204, 206, 208, 210, 212, and/or 214 run on native device frameworks, and are available for download onto the system 200 running an operating system 202 such as Android or iOS.

In some implementations, one or more of the above identified data elements or modules of the system 200 for evaluating an effect of one or more perturbations and/or therapeutic candidate compounds on a cell are stored in one or more of the previously described memory devices, and correspond to a set of instructions for performing a function described above. The above-identified data, modules or programs (e.g., sets of instructions) need not be implemented as separate software programs, procedures or modules, and thus various subsets of these modules may be combined or otherwise re-arranged in various implementations. In some implementations, the memory 298 and/or 299 optionally stores a subset of the modules and data structures identified above. Furthermore, in some embodiments the memory 298 and/or 299 stores additional modules and data structures not described above.

In some embodiments, device 200 for evaluating an effect of one or more perturbations and/or therapeutic candidate compounds on a cell is a smart phone (e.g., an iPHONE), laptop, tablet computer, desktop computer, or other form of electronic device. In some embodiments, the device 200 is not mobile. In some embodiments, the device 200 is mobile.

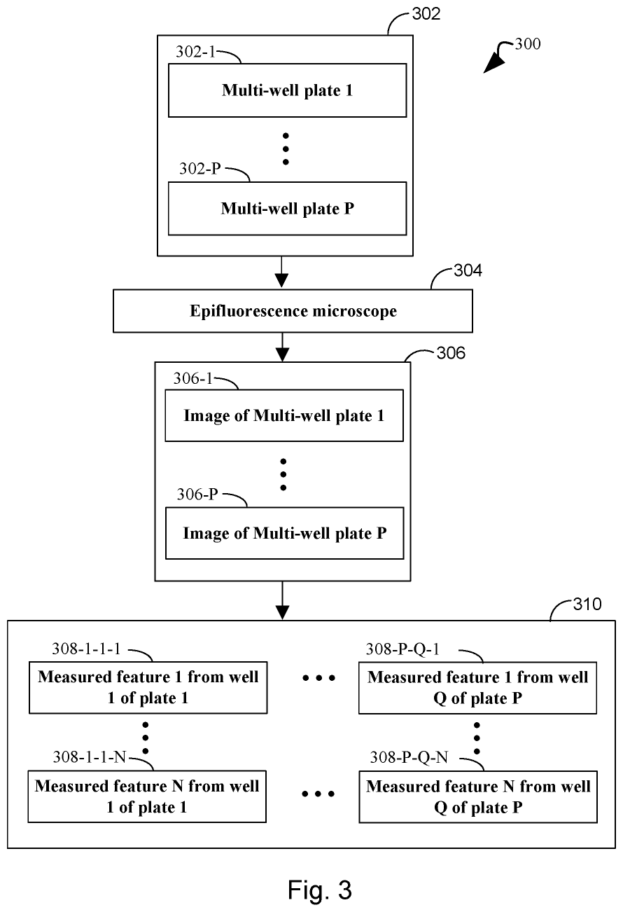

Referring to FIG. 3, in some embodiments, the present disclosure relies upon the acquisition of a data set 222 that includes measurements of a plurality of features 308 (e.g., plate control feature measurements 226, assay control feature measurements 230, and test feature measurements 234) for cell contexts exposed to one or more perturbation and/or candidate therapeutic compound, in one or more replicates, in one or more cell contexts, at one or more concentrations, e.g., As an example, each candidate compound i in a plurality of M compounds is introduced into wells of a multi-well plate 302 at each of k concentrations for each of l perturbed cell contexts in j instances, resulting in X wells containing compound i, where X=(j)*(k)*(l). N features are then measured from each well {1 . . . Q} of each multi-well plate {1 . . . P}, resulting in N*M*X* feature measurements for the candidate compounds.

In some embodiments, referring to FIG. 3, these feature measurements are acquired by capturing images 306 of the multi-well plates using, for example, epifluorescence microscopy using an epifluorescence microscope 304. The images 306 are then used as a basis for obtaining the measurements of the N different features from each of the wells in the multi-well plates, thereby forming dataset 310 (e.g., data set 222). Data set 310 is then used to generate multidimensional vectors (e.g., plate control vectors 246, assay control vectors 250, and test vectors 254) which, in turn, are used to evaluate the effects of a perturbation and/or candidate therapeutic compound on a cell context.

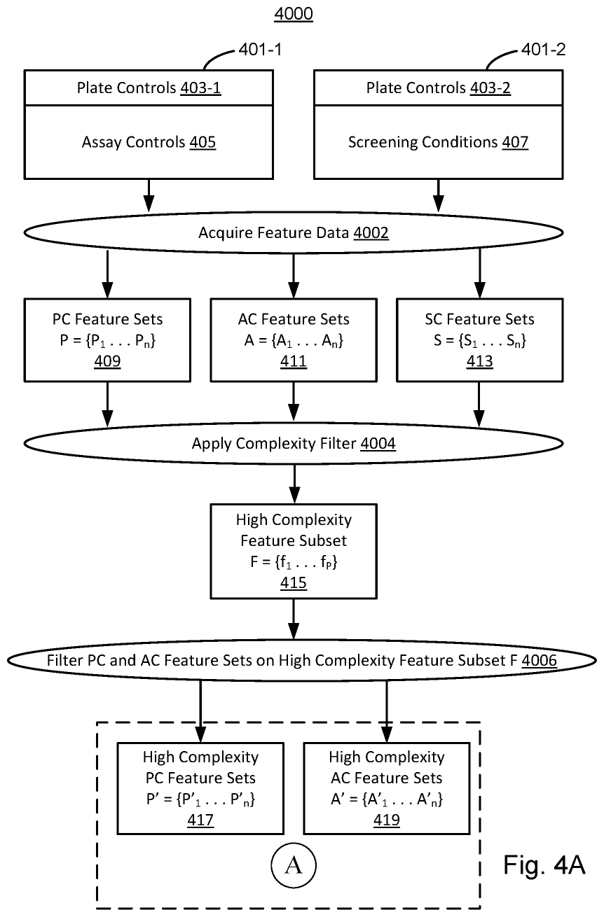

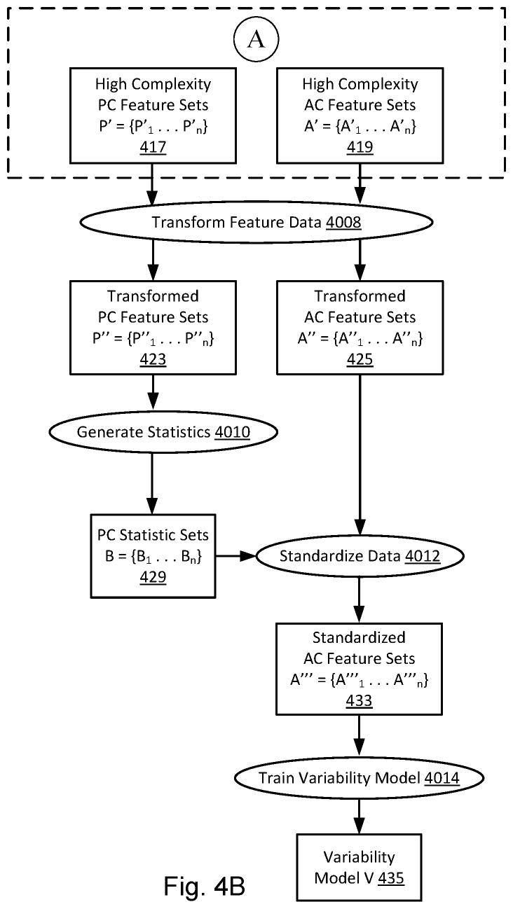

Now that details of a system 200 for evaluating an effect of one or more perturbations and/or therapeutic candidate compounds on a cell have been disclosed, details regarding a processes and features of the system, in accordance with an embodiment of the present disclosure, are disclosed below. Example processes are also described with reference to FIGS. 4A-4B, 5A-5B, 6A-6C, and 7A-7B. In some embodiments, such processes and features of the system are carried out by modules 204, 206, 208, 210, 212, and/or 214, as illustrated in FIG. 2. Referring to these methods, the systems described herein (e.g., system 200) include instructions for performing the methods for evaluating an effect of one or more perturbations and/or therapeutic candidate compounds on a cell.

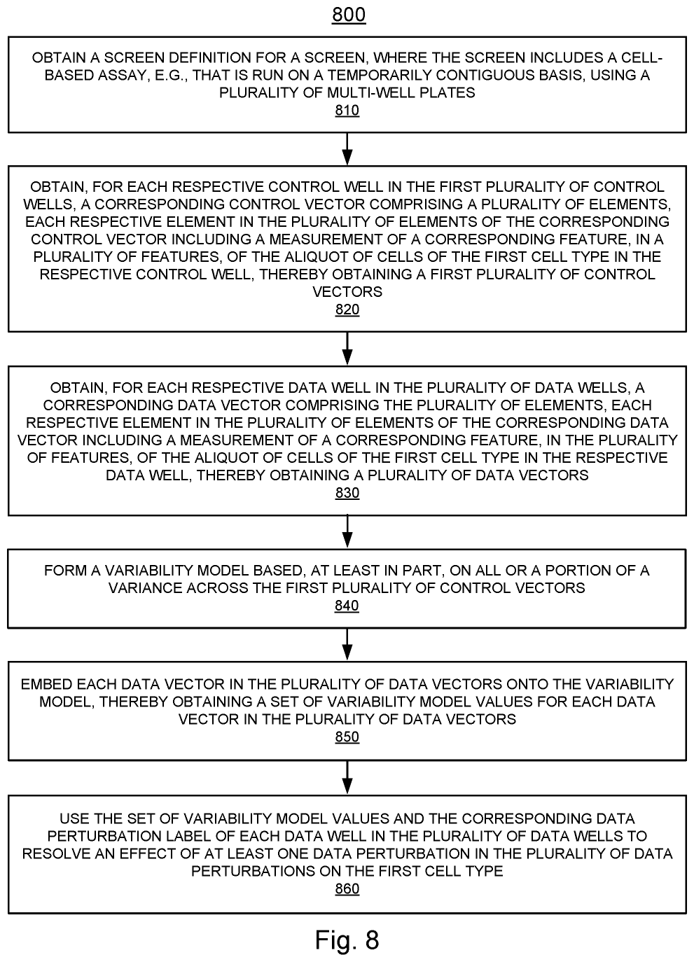

Referring now to FIG. 8, which depicts an example method 800 for evaluating an effect of one or more perturbations on cells of a first cell type, in accordance with various embodiments. Method 800 may be thought of as an overarching method, in which many of aspects of the method are described in greater detail with reference to the procedures in FIGS. 4A-4B, 5A-5B, 6A-6C, and 7A-7B. In some embodiments, aspects of method 800 are performed by a computer system such as computer system 200. In some embodiments, aspects of method 800 may be embedded as instructions on non-transitory computer readable media, which when executed cause a computer system, such as computer system 200 to perform the procedures.

At 810 of method 800, in various embodiments, the method includes obtaining a screen definition for a screen, where the screen includes a cell-based assay, e.g., that is run on a temporarily contiguous basis, using a plurality of multi-well plates. The screen definition identifies a first plurality of control wells and a plurality of data wells in the plurality of multi-well plates. Each respective control well in the first plurality of control wells is labeled with a control perturbation label corresponding to a control perturbation in a first plurality of control perturbations that is independently included in the respective control well. Each respective data well in the plurality of data wells is labeled with a data perturbation label corresponding to a data perturbation in a plurality of data perturbations that is independently included in the respective data well. An aliquot of cells of the first cell type is included in each control well in the first plurality of control wells and in each data well in the plurality of data wells.

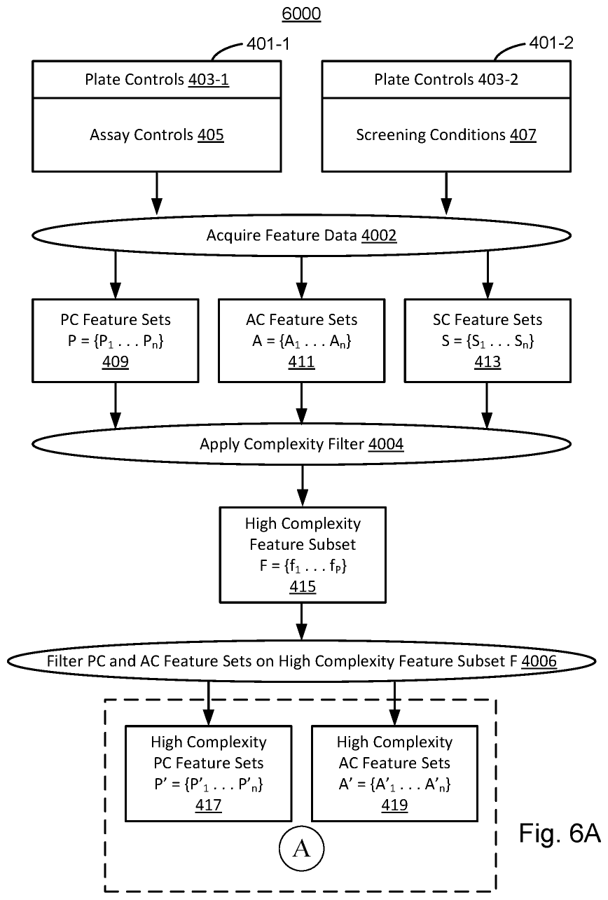

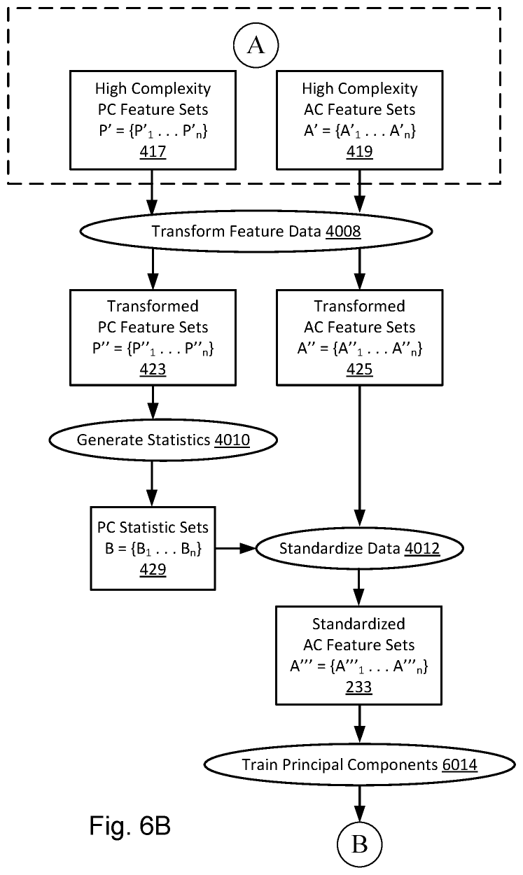

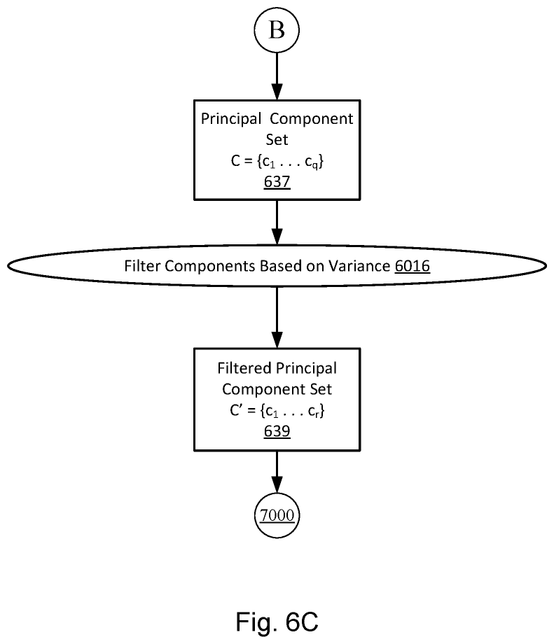

At 820 of method 800, in various embodiments, the method also includes obtaining, for each respective control well in the first plurality of control wells, a corresponding control vector comprising a plurality of elements, each respective element in the plurality of elements of the corresponding control vector including a measurement of a corresponding feature, in a plurality of features, of the aliquot of cells of the first cell type in the respective control well, thereby obtaining a first plurality of control vectors (e.g., assay control vectors 248 formed from assay control data 228 by feature vector control module 204, as illustrated in FIG. 2, and/or assay feature sets 411, as illustrated in FIGS. 4A and 6A, respectively).

At 830 of method 800, in various embodiments, the method also includes obtaining, for each respective data well in the plurality of data wells, a corresponding data vector comprising the plurality of elements, each respective element in the plurality of elements of the corresponding data vector including a measurement of a corresponding feature, in the plurality of features, of the aliquot of cells of the first cell type in the respective data well, thereby obtaining a plurality of data vectors (e.g., data vectors 252 formed from test data 232 by feature vector control module 204, as illustrated in FIG. 2, and/or screening condition feature sets 413, as illustrated in FIGS. 4A, 5A, and 6A, respectively).

In some embodiments, the underlying data (e.g., previously collected feature measurements) are obtained and vectors are constructed therefrom, e.g., by combining data received for individual feature measurements. In some embodiments, feature measurements are collected directly by the system (e.g., system 200), e.g., the system includes instructions for processing images acquired of microwell plates. In some embodiments, the vectors and/or underlying data for the vectors is obtained from a remote source, e.g., over network 296 via network interface 295.

At 840 of method 800, in various embodiments, the method then includes forming a variability model based, at least in part, on all or a portion of a variance across the first plurality of control vectors (e.g., training (4014) of variability model 435 using standardized assay control feature sets 433, as illustrated in FIG. 4B).

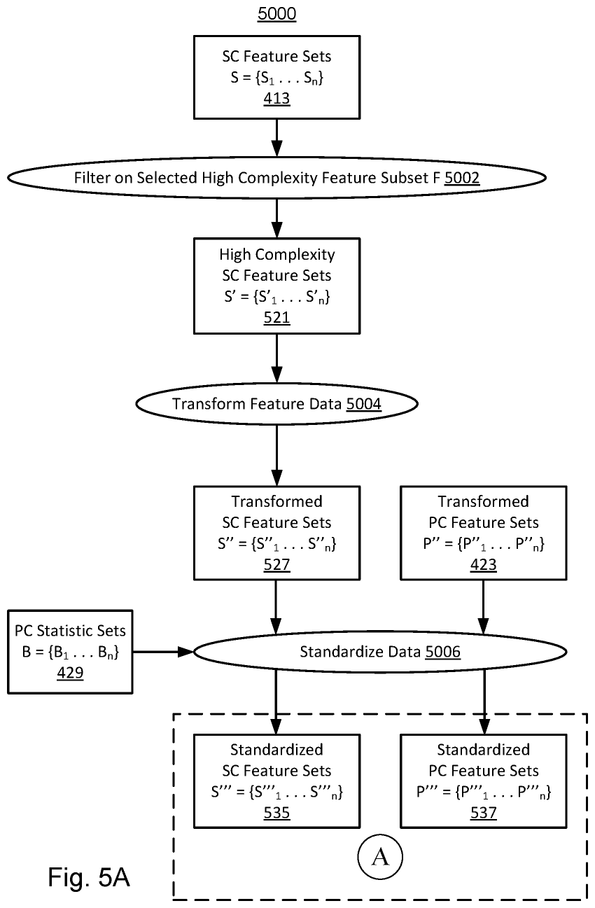

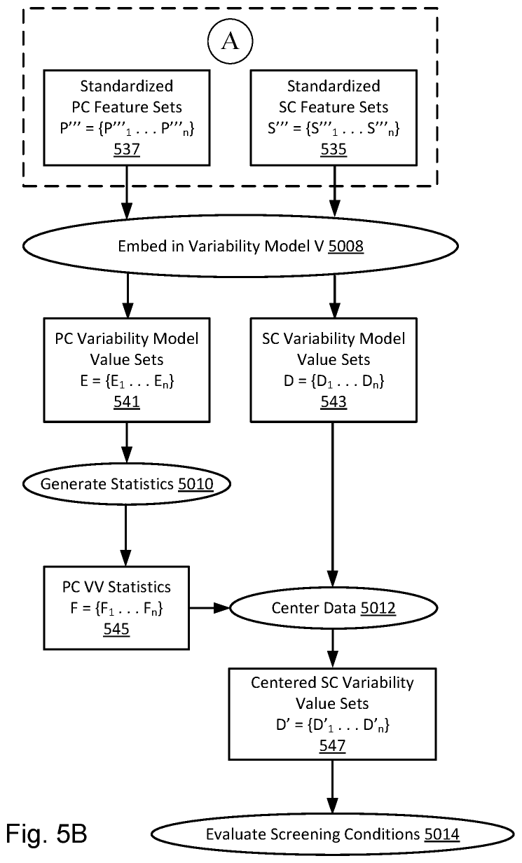

FIGS. 5A and 5B collectively illustrate an example process 5000 for evaluating an effect of one or more perturbations on cells using a trained variability model, in accordance with various embodiments of the present disclosure.

FIGS. 6A, 6B, and 6C collectively illustrate an example process 6000 for training principal components for use in evaluating an effect of one or more perturbations on cells, in accordance with various embodiments of the present disclosure. Many aspects of this process are the same as those illustrated in FIGS. 4A and 4B.

At 850 of method 800, in various embodiments, the method then includes embedding 5008 each data vector in the plurality of data vectors onto the variability model, thereby obtaining a set of variability model values for each data vector in the plurality of data vectors (e.g., embedding 4008 standardized screening condition feature sets 535 and standardized plate control features sets 537 onto variability model 435, to form plate control variability model value sets 541 and screening condition variability model value sets 543, as illustrated in FIG. 5B). From plate control variability model value sets 541 statistics are determined/generated 5010 (in a similar manner to 4010 of FIG. 4B), such as measure of central tendency and standard deviation, for each features across each well in a multi-well plate to generate a plate control VV statistic set 545 for the multi-well plate, as illustrated in FIG. 5B, and then using the plate control statistic set 545 to normalize/standardize/center 5012 screening condition variability model value sets 543 into centered screening condition variability model value sets 547, as illustrated in FIG. 5B).

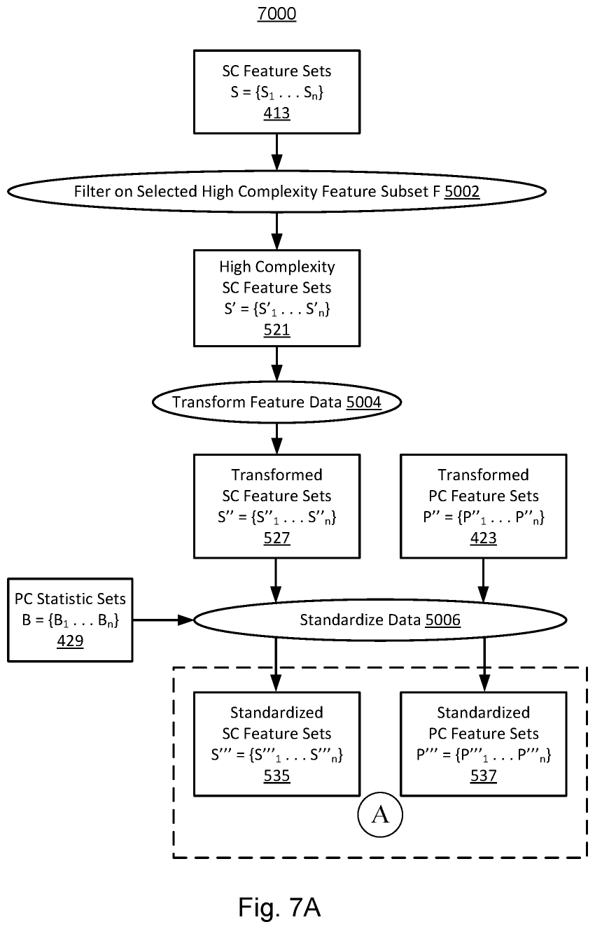

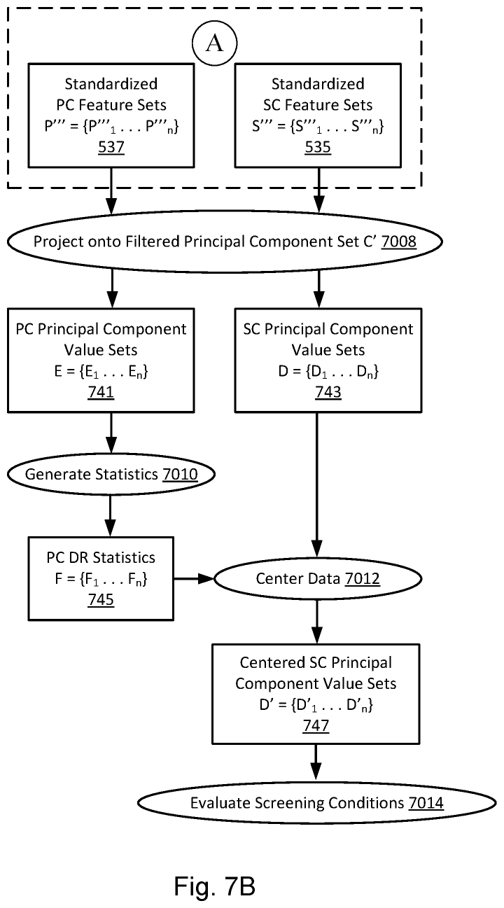

Alternatively, in some embodiments method then includes 7008 (FIG. 7C) each data vector in the plurality of data vectors onto a filtered principal component set c′ (639 of FIG. 6C), thereby obtaining a set of PC principal component model values for each data vector in the plurality of data vectors (e.g., embedding 7008 standardized screening condition feature sets 535 and standardized plate control features sets 537 onto set 639, to form plate control principal component value sets 741 and screening condition principal component value sets 743, as illustrated in FIG. 7B). From plate control screening condition value sets 741 statistics are determined/generated 7010 (in a similar manner to 4010 of FIG. 4B), such as measure of central tendency and standard deviation, for each features across each well in a multi-well plate to generate a plate control DR statistic set 745 for the multi-well plate, as illustrated in FIG. 7B, and then using the plate control DR statistic set 745 to normalize/standardize/center 7012 screening condition principal component value sets 743 into centered screening condition principal component value sets 747, as illustrated in FIG. 7B).

At 860 of method 800, in various embodiments, the method then includes using the set of variability model values and the corresponding data perturbation label of each data well in the plurality of data wells to resolve an effect of at least one data perturbation in the plurality of data perturbations on the first cell type (e.g., evaluating (5014) centered screening condition variability model value sets 547, as illustrated in FIG. 5B).

Additionally or alternatively, in some embodiments, method then includes using the set of principal component values and the corresponding data perturbation label of each data well in the plurality of data wells to resolve an effect of at least one data perturbation in the plurality of data perturbations on the first cell type (e.g., evaluating (7014) centered screening condition variability model value sets 747, as illustrated in FIG. 7B).

In some embodiments, as illustrated by process 4000 of FIGS. 4A and 4B, the first plurality of control wells is in a first subset of the plurality of plates, the plurality of data wells is in a second subset of the plurality of plates, and the second subset of the plurality of plates is other than the first subset of the plurality of plates (e.g., assay controls 405 and screening conditions 407 are in separate multi-well plates 401 (e.g., 401-1, 401-2)). In some embodiments, the first plurality of control wells consists of between 200 control wells and 1500 control wells in the second subset of the plurality of plates. In some embodiments, each control perturbation in the first plurality of control perturbations is a different siRNA.

In some embodiments, the screen definition further includes a second plurality of control wells (e.g., corresponding to plate controls 403, as illustrated in FIG. 4A). There is an aliquot of cells of the first cell type (e.g., the same cell type as in the first plurality of control wells and the data wells) in each control well in the second plurality of wells. The second plurality of control wells is present in each plate in the plurality of plates (e.g., each of multi-well plates 401 include the same set of plate controls 403, as illustrated in FIG. 4A). Each respective control well in the second plurality of control wells is labeled with a control perturbation label corresponding to a control perturbation in a second plurality of control perturbations that is independently included in the respective control well and the second plurality of control wells collectively represents each control perturbation in the second plurality of control perturbations. Accordingly, in some embodiments, method 800 includes, for each respective plate in the plurality of plates, obtaining, for each respective control well in the second plurality of control wells of the respective plate, a corresponding normalization vector comprising the plurality of elements (e.g., plate control feature sets 409 in FIG. 4A), each respective element in the plurality of elements of the normalization vector including a measurement of a corresponding feature, in the plurality of features, of the aliquot of cells of the first cell type in the respective control well, thereby obtaining a plurality of normalization vectors, and using the plurality of normalization vectors to normalize a set of data wells in the plurality of data wells that are in the respective plate prior to the obtaining (e.g., by determining/generating statistics 4010, such as measure of central tendency and standard deviation, for each features across each well in a multi-well plate to generate a plate control statistic set 429 for the multi-well plate, as illustrated in FIG. 4B, and then using the plate control statistic set 429 to normalize transformed screening condition feature sets 527, as illustrated in FIG. 5A).

In some embodiments, using the plurality of normalization vectors to normalize the set of data wells in the plurality of data wells that are in the respective plate includes computing a first measure of central tendency for each respective feature in the plurality of features across each corresponding normalization vector in the plurality of normalization features thereby forming a first plurality of measures of central tendency, each first measure of central tendency in the first plurality of measures of central tendency for a feature in the plurality of features. Then for each respective data well in the set of data wells in the plurality of data wells that are in the respective plate, for each respective feature in the plurality of features, subtracting a measured value for the respective feature by the first measure of central tendency corresponding to the respective feature and dividing the measured value for the respective feature by a standard deviation in measurement of the respective feature across the plurality of normalization vectors.

In some embodiments, the measure of central tendency of the measurement of the different feature is an arithmetic mean, weighted mean, midrange, midhinge, trimean, Winsorized mean, median, or mode of the different feature across a plurality of control aliquots of the cells representing the respective control perturbation in between a plurality of corresponding wells in the plurality of wells.

In some embodiments, the variability model is a plurality of dimension reduction components, and method 800 includes obtaining, for each respective control well in the second plurality of control wells of the respective plate, a corresponding dimension reduction normalization vector comprising a dimension reduction component value for each respective dimension reduction component, in the plurality of dimension reduction components by projecting the measurement of the corresponding features, in the plurality of features for the respective plate, specified by the respective dimension reduction component onto the respective dimension reduction component thereby obtaining a plurality of dimension reduction normalization vectors, and using the plurality of dimension reduction normalization vectors to standardize the set of data wells (4012 of FIG. 4B, 5006 of FIG. 5A) in the plurality of data wells that are in the respective plate prior to the computing.

In some embodiments, using the plurality of dimension reduction normalization vectors to standardize the set of data wells 4012/5006 in the plurality of data wells that are in the respective plate includes computing a second measure of central tendency for each respective dimension reduction component in the plurality of dimension reduction components across each corresponding dimension reduction normalization vector in the plurality of dimension reduction normalization vectors thereby forming a plurality of second measures of central tendency, each second measure of central tendency in the plurality of second measures of central tendency for a dimension reduction component in the plurality of dimension reduction components. Then, for each respective data well in the set of data wells in the respective plate, for each respective dimension reduction component in the plurality of dimension reduction components, subtracting a measured value for the respective dimension reduction component by the second measure of central tendency corresponding to the respective dimension reduction component across the plurality of dimension reduction normalization vectors.

In some embodiments, method 800 includes, prior to the forming variability model 4014, pruning the plurality of features by removing from the plurality of features each feature in the plurality of features that fails to satisfy a diversity threshold across the first plurality of control vectors (e.g., by applying a complexity filter 4004, as illustrated in FIG. 4A, to one or more of plate control (PC) feature sets 409, assay control (AC) feature sets 411, and screening conditions (SC) feature set 413 and identifying features that do not provide a threshold amount of variation across the corresponding measurements, thereby forming high complexity feature subset 415, which can be applied to each feature set 409, 411, and 413, to form (via filtering 4006, 5002) high complexity feature sets 417, 419, and 521 as illustrated in FIGS. 4A, 5A, and 6A).

In some embodiments, the variability model is a plurality of dimension reduction components, and the plurality of dimension reduction components account for at least ninety percent of the variance of the plurality of features across the first plurality of control vectors. For example, as illustrated in FIGS. 6A/6B and 7A/7B, in some embodiments, the dimension reduction components are principal components 637, which are pruned/filtered based on variance 6016 to provide filtered principal component set 639, containing the principal components that account for the greatest variance in the training set, e.g., at least 90%, 95%, 99%, 99.9%, 99.99%, or more variance. Filtered principal component sets 639 may be provided for use in process 7000 illustrated in FIGS. 7A and 7B. In some embodiments, the variability model is a plurality of dimension reduction components, and wherein the plurality of dimension reduction components account for at least ninety-nine percent of the variance of the plurality of features across the first plurality of control vectors.

In some embodiments, the plurality of dimension reduction components is a plurality of principal components and wherein the forming (840 of FIG. 8) comprises applying principal component analysis to the plurality of features across the first plurality of control vectors (e.g., training (6014) principal components against standardized assay control feature sets 233, as illustrated in FIG. 6B).

In some embodiments, for each respective control well in the first plurality of control wells, the plurality of elements of the corresponding control vector further comprises, for each respective feature in the plurality of features a transform, selected from among a set of transforms in accordance with a feature transform lookup table, of the measurement of the respective feature in the respective control well, and for each respective data well in the plurality of data wells, the plurality of elements of the corresponding data vector further comprises, for each respective feature in the plurality of features, a transform, selected from among a set of transforms in accordance with the feature transform lookup table, of the measurement of the respective feature in the respective data well. For instance, transforming (4008, 5004) feature sets 419 and 521, as illustrated in FIGS. 4B and 5A, respectively into transformed PC features sets 423 and transformed AC feature sets 425 (FIGS. 4B and 5A) or transformed screening conditions features sets 527 (FIG. 5A). In some embodiments, for each respective control well in the second plurality of control wells, the plurality of elements of the corresponding normalization vector further comprises, for each respective feature in the plurality of features, a transform, selected from among a set of transforms in accordance with a feature transform lookup table, of the measurement of the respective feature in the respective control well. For instance, transforming (4008) feature set 417, as illustrated in FIG. 4B.

In some embodiments, a transform in the set of transforms is a natural log transform of the measurement of the respective feature or a natural log transform of the measurement of the respective feature adjusted by a fixed increment. In some embodiments, the set of transforms comprises (i) a natural log transform of the measurement of the respective feature, (ii) a natural log transform of the measurement of the respective feature adjusted by a first fixed increment, and (iii) a natural log transform of the measurement of the respective feature adjusted by a second fixed increment. In some embodiments, the first fixed increment is 0.1 and the second fixed increment is 1.

In some embodiments, the first measure of central tendency for a respective feature is an arithmetic mean, weighted mean, midrange, midhinge, trimean, Winsorized mean, median, or mode of the respective feature across the plurality of normalization vectors. In some embodiments, the second measure of central tendency for a respective dimension reduction component is an arithmetic mean, weighted mean, midrange, midhinge, trimean, Winsorized mean, median, or mode of the respective dimension reduction component across the plurality of dimension reduction components.

In some embodiments, each feature in the plurality of features represents a color, texture, or size of the cell or an enumerated portion of the cell.

In some embodiments, obtaining the control and data vectors (e.g., acquiring feature data 4002, as illustrated in FIG. 4A) includes imaging a corresponding well in the plurality of data wells or in the plurality of control wells to form a corresponding two-dimensional pixelated image having a corresponding plurality of native pixel values and wherein a different feature in the plurality of features arises as a result of a convolution or a series convolutions and pooling operators run against native pixel values in the corresponding plurality of native pixel values of the corresponding two-dimensional pixelated image.

In some embodiments, the aliquot of the cells of a respective control well is exposed to the respective control perturbation in the respective control well for at least one hour prior to obtaining the measurement of each feature in the plurality of features. In some embodiments, the aliquot of the cells of a respective control well is exposed to the respective control perturbation in the respective control well for at least one hour, two hours, three hours, one day, two days, three days, four days, or five days prior to obtaining the measurement of each feature in the plurality of features. In some embodiments, each control perturbation in the plurality of control perturbations is a different siRNA.

In some embodiments, the aliquot of the cells of a respective data well is exposed to a data perturbation, in a plurality of data perturbations, in the respective data well for at least one hour prior to obtaining the measurement of each feature in the plurality of features. In some embodiments, the aliquot of the cells of a respective data well is exposed to a data perturbation, in a plurality of data perturbations, in the respective data well for at least one hour, two hours, three hours, one day, two days, three days, four days, or five days prior to obtaining the measurement of each feature in the plurality of features. In some embodiments, each data perturbation in the plurality of data perturbations is a different siRNA.

In some embodiments, the variability model is a plurality of dimension reduction components that consists of between 100 dimension reduction components and 300 dimension reduction components. In some embodiments, the variability model is a neural network.

In some embodiments, each feature in the plurality of features is an optical feature that is optically measured. In some embodiments, a first subset of the plurality of features are optical features that are optically measured and a second subset of the plurality of features are non-optical features. In some embodiments, each feature in the plurality of features is a feature that is non-optically measured. The skilled artisan will know of other feature measurements suitable for use in the present methods, for example, as described in detail below.

In some embodiments, each feature in the plurality of features represents a color, texture, or size of the cell or an enumerated portion of the cell. In some embodiments, obtaining the feature measurements includes imaging a corresponding well in the plurality of wells to form a corresponding two-dimensional pixelated image having a corresponding plurality of native pixel values and where a different feature in the plurality of features of the obtaining arises as a result of a convolution or a series convolutions and pooling operators run against native pixel values in the corresponding plurality of native pixel values of the corresponding two-dimensional pixelated image. That is, in some embodiments, the plurality of features includes latent features of an image of the respective well in the multi-well plate.

In some embodiments, the plurality of control perturbations comprises a toxin, a cytokine, a predetermined drug, a siRNA, an sgRNA, a cell culture condition, or a genetic modification. In some embodiments, each data perturbation in the plurality of data perturbations is a toxin, a cytokine, a predetermined drug, a siRNA, an sgRNA, a cell culture condition, or a genetic modification.

In some embodiments, the set of data perturbations consists of a plurality of target siRNA that directly affect (e.g., suppress) expression of a gene associated with the test state (4036). For instance, in some embodiments, a perturbation being tested partially disrupts the expression of a gene or a function of a gene product and the set of data perturbations includes different siRNA that suppress expression of the gene (e.g., by targeting different sequences of the gene).

In some embodiments, the set of data perturbations includes a plurality of target siRNA that directly affect expression of one of a plurality of genes corresponding to proteins in the same pathway associated with the test state, e.g., a metabolic or signaling pathway related to a disease of interest. For instance, in some embodiments, a perturbation being tested partially disrupts the function of a pathway the set of data perturbations includes different siRNA that target genes encoding different proteins participating in the pathway. In some embodiments, multiple siRNA are used to target any one of the genes involved in the pathway (e.g., by targeting different sequences of the gene).

In some embodiments, the set of data perturbations includes a small interfering RNA (siRNA) that specifically recognizes a particular gene in the aliquot of first cells. Each siRNA is a double-stranded RNA molecule, 20-25 base pairs in length that interferes with the expression of a specific gene with a complementary nucleotide sequence by degrading mRNA after transcription preventing translation of the gene. An siRNA is an RNA duplex that can reduce gene expression through enzymatic cleavage of a target mRNA mediated by the RNA induced silencing complex (RISC). An siRNA has the ability to inhibit targeted genes with near specificity. See, Agrawal et al., 2003, “RNA interference: biology, mechanism, and applications,” Microbiol Mol Biol Rev. 67: 657-85; and Reynolds et al., 2004, “Rational siRNA design for RNA interference,” Nature Biotechnology 22, 326-330, each of which is hereby incorporated by reference. In some such embodiments, the perturbation is achieved by transfecting the siRNA into the cells, DNA-vector mediated production, or viral-mediated siRNA synthesis. See, for example, Paddison et al., 2002, “Short hairpin RNAs (shRNAs) induce sequence-specific silencing in mammalian cells,” Genes Dev. 16:948-958; Sui et al., 2002, A DNA vector-based RNAi technology to suppress gene expression in mammalian cells,” Proc Natl Acad Sci USA 99:5515-5520; Brummelkamp et al., 2002, “A system for stable expression of short interfering RNAs in mammalian cells,” Science 296:550-553; Paddison et al., 2004, “Short hairpin activated gene silencing in mammalian cells,” Methods Mol Biol 265:85-100; Wong et al. 2003, “CIITAregulated plexin-A1 affects T-cell-dendritic cell interactions, Nat Immunol 2003, 4:891-898; Tomar et al., 2003, “Use of adeno-associated viral vector for delivery of small interfering RNA. Oncogene 22:5712-5715; Rubinson et al., 2003 “A lentivirus-based system to functionally silence genes in primary mammalian cells, stem cells and transgenic mice by RNA interference,” Nat Genet 33:401-406; Moore et al., 2005, “Stable inhibition of hepatitis B virus proteins by small interfering RNA expressed from viral vectors,” J Gene Med; and Tran et al., 2003, “Expressing functional siRNAs in mammalian cells using convergent transcription, BMC Biotechnol 3:21; each of which is hereby incorporated by reference.

In some embodiments, the set of data perturbations includes a material that is taken directly from cells or from fluids, tissues or organs of patients exhibiting a disease of interest (e.g., synovial fluid from rheumatoid arthritis patients). In some embodiments this material is referred to as a “conditioned medium.” For instance, by way of example, in some embodiments the material is a synovial tissue explant (See, Beekhuizen et al., 2011, “Osteoarthritic synovial tissue inhibition of proteoglycan production in human osteoarthritic knee cartilage: establishment and characterization of a long-term cartilage-synovium coculture,” Osteoarthritis 63, 1918, which is hereby incorporated by reference) that is either immediately used as a test perturbation or is cultured for a predetermined period of time prior to use as a perturbation. By way of another example, in some embodiments the material is mesenchymal stem cells (MSCs) that have been isolated and cultured from heparinized femoral-shaft marrow aspirate of human patients undergoing total hip arthroplasty, seeded in cell medium (e.g., Dulbecco's Modified Eagle Medium). See, Buul, 2012, “Mesenchymal stem cells secrete factors that inhibit inflammatory processes in short-term osteoarthritic synovium and cartilage explant culture,” Osteoarthritis and Cartilage 20, 1186, which is hereby incorporated by reference. See also, Kay et al., 2017, “Mesenchymal Stem Cell-Conditioned Medium Reduces Disease Severity and Immune Responses in Inflammatory Arthritis,” Nature 7, 18019, which is hereby incorporated by reference, for an example of the preparation of a condition medium in the form of murine MSCs isolated form BALB/C mice. By way of still another example, in some embodiments, the material is human synovial explants or cartilage explants obtained as surgical waste material from patients undergoing knee replacement surgery. In such embodiments, the perturbation is the material extracted directly from cells or from fluids, tissues or organs of patients exhibiting a disease of interest that is either used immediately after extraction, or after the material has been cultured for a period of time. In some embodiments, the material is cultured in the presence of factors that are intended to stimulate the material. For instance, in the case where the material is mesenchymal stem cells, in some embodiments, by way of example, the material is cultured in the presence of TNFα and IFNγ to stimulate the secretion of immunomodulatory factors by MSCs. See, Buul, 2012, Osteoarthritis and Cartilage 20, 1186, which is hereby incorporated by reference. For another example of the preparation of conditioned medium, see Martin, 1981, “Isolation of a pluripotent cell line from early mouse embryos cultured in medium conditioned by teratocarcinoma stem cells,” PNAS 78, 7634, which is hereby incorporated by reference.

In some embodiments, the set of data perturbations includes a short hairpin RNA (shRNA). See, Taxman et al., 2006, “Criteria for effective design, construction, and gene knockdown by shRNA vectors,” BMC Biotechnology 6:7 (2006), which is hereby incorporated by reference. In some such embodiments, the perturbation is achieved by DNA-vector mediated production, or viral-mediated siRNA synthesis as generally discussed in the references cited above for siRNA.

In some embodiments, the set of data perturbations includes a single guide RNA (sgRNA) used in the context of palindromic repeat (CRISPR) technology. See, Sander and Young, 2014, “CRISPR-Cas systems for editing, regulating and targeting genomes,” Nature Biotechnology 32, 347-355, hereby incorporated by reference, in which a catalytically-dead Cas9 (usually denoted as dCas9) protein lacking endonuclease activity to regulate genes in an RNA-guided manner. Targeting specificity is determined by complementary base-pairing of a single guide RNA (sgRNA) to the genomic loci. sgRNA is a chimeric noncoding RNA that can be subdivided into three regions: a 20 nt base-pairing sequence, a 42 nt dCas9-binding hairpin and a 40 nt terminator. In some embodiments, when designing a synthetic sgRNA for use as a perturbation, only the 20 nt base-pairing sequence is modified from the overall template. Additionally, in some embodiments, secondary variables are considered such as off target effects and maintenance of the dCas9-binding hairpin structure. In some embodiments, the Cas9 is rendered catalytically inactive by introducing point mutations in the two catalytic residues (D10A and H840A) of the gene encoding Cas9. See Jinek et al., 2012, “A Programmable Dual-RNA-Guided DNA Endonuclease in Adaptive Bacterial Immunity,” Science 337, (6096), 816, which is hereby incorporated by reference. In doing so, dCas9 is unable to cleave dsDNA but retains the ability to target DNA. In some such embodiments, the perturbation is achieved by DNA-vector mediated production, or viral-mediated sgRNA synthesis as generally discussed in the references cited above for siRNA.

In some embodiments, the set of data perturbations includes a cytokine or mixture of cytokines. See Heike and Nakahata, 2002, “Ex vivo expansion of hematopoietic stem cells by cytokines,” Biochim Biophys Acta 1592, 313-321, which is hereby incorporated by reference, for suitable assays for exposing entities to perturbations in the form of cytokines (e.g., in vitro assays such as long-term culture-initiating cell (LTCIC) assay, cobblestone area-forming cell (CAFC) assay, high proliferative potential colony-forming cell (HPP-CFC) assay, and colony-forming unit-blast (CFU-BI) assay, as well as in vivo assays using animal models). In some embodiments entities are exposed to perturbations in the form of cytokines (e.g., lymphokines, chemokines, interferons, tumor necrosis factors, etc.). In some embodiments entities are exposed to perturbations in the form of lymphokines (e.g., Interleukin 2, Interleukin 3, Interleukin 4, Interleukin 5, Interleukin 6, granulocyte-macrophage colony-stimulating factor, interferon gamma, etc.). In some embodiments entities are exposed to perturbations in the form of chemokines such as homeostatic chemokines (e.g., CCL14, CCL19, CCL20, CCL21, CCL25, CCL27, CXCL12, CXCL13, etc.) and/or inflammatory chemokines (e.g., CXCL-8, CCL2, CCL3, CCL4, CCL5, CCL11, CXCL10). In some embodiments entities are exposed to perturbations in the form of interferons (IFN) such as a type I IFN (e.g., IFN-α, IFN-β, IFN-ε, IFN-κ and IFN-ω), a type II IFN (e.g., IFN-γ), or a type III IFN. In some embodiments entities are exposed to perturbations in the form of tumor necrosis factors such as TNFα or TNF alpha.

In some embodiments, the set of data perturbations includes a compound. In some such embodiments the activity of such a compound against the cells of the first cell type is assayed using a phosphoflow technique such as one disclosed in Krutzik et al., 2008, “High-content single-cell drug screening with phosphospecific flow cytometry,” Nature Chemical Biology 4, 132-142, which is hereby incorporated by reference. In some embodiments the test perturbation is a compound having a molecular weight of less than 2000 Daltons. In some embodiments, the test perturbation is any organic compound having a molecular weight of less than 2000 Daltons, of less than 4000 Daltons, of less than 6000 Daltons, of less than 8000 Daltons, of less than 10000 Daltons, or less than 20000 Daltons.

In some embodiments, the set of data perturbations includes a chemical compound that satisfies the Lipinski rule of five criteria. In some embodiments, the test perturbation is an organic compound that satisfies two or more rules, three or more rules, or all four rules of the Lipinski's Rule of Five: (i) not more than five hydrogen bond donors (e.g., OH and NH groups), (ii) not more than ten hydrogen bond acceptors (e.g., N and O), (iii) a molecular weight under 500 Daltons, and (iv) a LogP under 5. The “Rule of Five” is so called because three of the four criteria involve the number five. See, Lipinski, 1997, “Experimental and computational approaches to estimate solubility and permeability in drug discovery and development settings,” Adv. Drug Del. Rev. 23, 3-26, which is hereby incorporated herein by reference in its entirety. In some embodiments, the test perturbation satisfies one or more criteria in addition to Lipinski's Rule of Five. For example, in some embodiments, the test perturbation is a compound with five or fewer aromatic rings, four or fewer aromatic rings, three or fewer aromatic rings, or two or fewer aromatic rings.

In some embodiments, the set of data perturbations includes a protein perturbation such as a peptide aptamer. Peptide aptamers are combinatorial protein reagents that bind to target proteins with a high specificity and a strong affinity. By so doing, they can modulate the function of their cognate targets. In some embodiments, a peptide aptamer comprises one (or more) conformationally constrained short variable peptide domains, attached at both ends to a protein scaffold. Because peptide aptamers introduce perturbations that are similar to those caused by therapeutic molecules, their use identifies and/or validates therapeutic targets with a higher confidence level than is typically provided by methods that act upon protein expression levels. The combinatorial nature of peptide aptamers enables them to ‘decorate’ numerous polymorphic protein surfaces, whose biological relevance can be inferred through characterization of the peptide aptamers. Bioactive aptamers that bind druggable surfaces can be used in displacement screening assays to identify small-molecule hits to the surfaces. See, for example, Baines and Colas, 2006, “Peptide Aptamers as guides for small-molecule drug discovery,” Drug Discovery Today 11, 334-341, which is hereby incorporated by reference. In some embodiments a test perturbation is a peptide aptamer, that is, an artificial protein selected or engineered to bind specific target molecules. In some such embodiments, a peptide aptamer comprises one or more peptide loops of variable sequence displayed by a protein scaffold. In some embodiments the peptide aptamer is isolated from a combinatorial library. In some embodiments such a combinatorial library isolate is further improved by directed mutation or rounds of variable region mutagenesis and selection. In some embodiments, libraries of peptide aptamers are used as “mutagens,” in which a library that expresses different peptide aptamers is introduced into a population of entities, for selection of a desired phenotype, and an identification of those aptamers that cause the desired phenotype.

In some embodiments, the set of data perturbations includes a peptide aptamer derivatized with one or more functional moieties that can cause specific postranslational modification of their target proteins, or change the subcellular localization of the targets. See, for example, Colas et al., 2000, “Targeted modification and transportation of cellular proteins,” Proc. Natl. Acad. Sci. USA. 97 (25): 13720-13725, which is hereby incorporated by reference. In some embodiments, the peptides that form the aptamer variable regions are synthesized as part of the same polypeptide chain as the scaffold and are constrained at their N and C termini by linkage to it. This double structural constraint decreases the diversity of the conformations that the variable regions can adopt. As a consequence, peptide aptamers can bind their targets tightly, with binding affinities comparable to those shown by antibodies (nanomolar range). Peptide aptamer scaffolds are typically small, ordered, soluble proteins. One such scaffold is Escherichia coli thioredoxin, the trxA gene product (TrxA). See, Reverdatto et al., 2015, “Peptide aptamers: development and applications,” Curr. Top. Med. Chem. 15 (12): 1082-1101, which is hereby incorporated by reference. In these molecules, a single peptide of variable sequence is displayed instead of the Gly-Pro motif in the TrxA -Cys-Gly-Pro-Cys- active site loop. Improvements to TrxA include substitution of serines for the flanking cysteines, which prevents possible formation of a disulfide bond at the base of the loop, introduction of a D26A substitution to reduce oligomerization, and optimization of codons for expression in human cells. Reverdatto et al., further discloses other scaffolds that can be used, as does Škrlec et al., 2015, “Non-immunoglobulin scaffolds: a focus on their targets,” Trends Biotechnol. 33 (7): 408-418, which is hereby incorporated by reference. In some embodiments, the peptide aptamers are selected yeast two-hybrid systems and/or combinatorial peptide libraries constructed by phage display and other surface display technologies such as mRNA display, ribosome display, bacterial display and yeast display (e.g., biopannings). In some embodiments, the perturbation is a peptide aptamer that uses a peptide in the MimoDB database. See Huang et al., 2011, “MimoDB 2.0: a mimotope database and beyond,” Nucleic Acids Research. 40 (1): D271-D277, which is hereby incorporated by reference.

In some embodiments, the set of data perturbations includes a peptide that selectively affects protein-protein interactions within cells of the first cell type. In some such embodiments this protein-protein interaction affects an intracellular signaling event. See, for example, Souroujon and Mochly-Rosen, 1998, “Peptide modulators of protein-protein interactions in intracellular signaling,” Nature Biotechnology 16, 919-924, which is hereby incorporated by reference.

In some embodiments, the set of data perturbations includes a nucleic acid perturbation such as a nucleic acid aptamer. Nucleic acid aptamers are short synthetic single-stranded oligonucleotides that specifically bind to various molecular targets such as small molecules, proteins, nucleic acids, and even cells and tissues. See, Ni el al., 2011, “Nucleic acid aptamers: clinical applications and promising new horizons,” Curr Med Chem 18(27), 4206, which is hereby incorporated by reference. In some instance nucleic acid aptamers are selected from a biopanning method such as SELEX (Systematic Evolution of Ligands by Exponential enrichment). See, Ellington and Szostak, 1990, “In vitro selection of RNA molecules that bind specific ligands,” Nature 346(6287), 818; and Tuerk and Gold, 1990, “Systematic evolution of ligands by exponential enrichment: RNA ligands to bacteriophage T4 DNA polymerase,” Science 249(4968), 505, each of which is hereby incorporated by reference. The SELEX screening method begins with a random sequence library of ssDNA or ssRNA that spans 20-100 nucleotides (nt) in length. The randomization of nucleic acid sequences provides a diversity of 4ⁿ, with n corresponding to the number of randomized bases. Diversities on the order of ˜10¹⁶ aptamers can typically generated and screened in the SELEX methods. Each random sequence region is flanked by constant sequences that is used for capture or priming. To overcome exonuclease degradation, aptamers can be chemically synthesized and capped with modified or inverted nucleotides to prevent terminal degradation. Modified oligonucleotides can also be incorporated within the aptamer, either during or after selection, for enhanced endonuclease stability. Some modified nucleotide triphosphates, particularly 2′-O-modified pyrimidines, can be efficiently incorporated into nucleic acid aptamer transcripts by T7 RNA polymerases. Common chemical modifications included during selection are 2′-amino pyrimidines and 2′-fluoro pyrimidines. See, Ni et al., 2011, “Nucleic acid aptamers: clinical applications and promising new horizons,” Curr Med Chem 18(27), 4206, which is hereby incorporated by reference.

In some embodiments, the set of data perturbations includes an antibody or other form of biologic. In some embodiments, a library of test perturbations is used, where each member of the library is a different antibody. In some such embodiments, the library of antibodies comprises 100 antibodies, 1000 antibodies, or ten thousand antibodies. In some such embodiments, libraries of antibodies are generated using phage display techniques such as those disclosed in Wu et al., 2010, “Therapeutic antibody targeting of individual Notch receptors,” Nature 464, 1052-1057, which is hereby incorporated by reference. In some embodiments, a library of test perturbations is used, where each member of the library is a different biologic. In some such embodiments, the library of biologics comprises 100 biologics, 1000 biologics, or ten thousand biologics. In some such embodiments, entities are exposed to perturbations in the form of antibodies. For instance, in some such embodiments, such antibodies selectively bind to a transmembrane protein expressed by the entities, causing a cascading signal that selectively regulates a transcriptional program within the cells of the first cell type. For instance, as disclosed in Wu et al., id., receptors within the Notch family are widely expressed transmembrane proteins that function as key conduits through which mammalian cells communicate to regulate cell fate and growth. Ligand binding triggers a conformational change in the receptor negative regulatory region (NRR) that enables ADAM (a disintegrin and metalloproteinases) protease cleavage at a juxtamembrane site that otherwise lies buried within the quiescent NRR. Subsequent intramembrane proteolysis catalyzed by the c-secretase complex liberates the intracellular domain (ICD) to initiate the downstream Notch transcriptional program. Thus, in some embodiments, the test perturbation is an antibody that is exposed to the cells of the first cell type thereby causing a selective change in the transcription of one or more genes within the cells.

In some embodiments, the set of data perturbations includes a zinc finger transcription factor. In some such embodiments, the zinc finger protein transcription factor is encoded into vector that is transformed into the cells of the first cell type, thereby causing the control of expression of one or more targeted genes within the cells of the first cell type. In some such embodiments, a sequence that is common to multiple (e.g., functionally related) genes in the cells of the first cell type is used by a perturbation in the form of a zinc finger protein in order to control the transcription of all these genes with a single perturbation in the form of a zinc finger transcription factor. In some embodiments, the perturbation in the form of a zinc finger transcription factor targets a family of related gene in the cells of the first cell type by targeting and modulating the expression of the endogenous transcription factors that control them. See, for example, Doyon, 2008, “Heritable targeted gene disruption in zebrafish using designed zinc-finger nucleases,” Nature Biotechnology 26, 702-708, which is hereby incorporated by reference.

In some embodiments, the set of data perturbations includes perturbation that build confidence around the specificity of a biological signal related to a specific disease or other form of biological signal under study, for example, a particular phenotype exhibited by the cells of the first cell type) by uniquely inhibiting a gene in a biological pathway that is proximal (related) to the disease (or other form of biological signal under study) while each control perturbation has effects of similar magnitude on genes of cells of the first cell type that are not proximal to the genes of the biological signal under study. As such, in some embodiments the set of data perturbations provide a biological effect by targeting genetic components of the cells of the first type associated with the biological signal (e.g., disease) under study whereas the control perturbations target genetic components of the cells that are not proximal to the biological signal under study.

In some embodiments, each perturbation in the set of data perturbations is an siRNA, an sgRNA, or an shRNA.

In some embodiments, the plurality of target siRNA consists of between 4 and 12 different target siRNA. In some embodiments, the plurality of test siRNA includes at least 3, 4, 5, 6, 7, 8, 9, 10, 11, 12, 13, 14, 15, 20, 25, 30, 35, 40, 45, 50, 75, 100, 250, or more test siRNA.

In some embodiments, the set of data perturbations comprises a toxin, a cytokine, a predetermined drug, a siRNA, an sgRNA, a cell culture condition, or a genetic modification other than a control perturbation.

### Cell Contexts

As described above, control states and test states each refer to an experimental condition that generally includes a cell context. In some embodiments, the cell contexts used in the control and test states are exposed to a perturbation, as described above. In some embodiments, the cell contexts used in the control and test states are perturbed (e.g., by exposure to a compound or physical condition and/or through mutation of the cellular genome), to represent a ‘diseased’ phenotype. Accordingly, in some embodiments, the control and test states are then exposed to a candidate therapeutic compound and/or physical conditions.

In some embodiments, a cell context is one or more cells that have been deposited within a well of a multi-well plate 302, such as a particular cell line, primary cells, or a co-culture system. In some embodiments, as described herein with reference to FIG. 3, in some embodiments, a compound in a compound library is exposed to a plurality of different perturbed cell contexts, e.g., at least two, three, four, five, six, seven, eight, nine, ten, or more perturbed cell contexts. In some embodiments, a compound in a compound library, is exposed to a single perturbed cell context (e.g., a single cell line or primary cell type).

Examples of cell types that are useful to be included in a cell context include, but are not limited to, U2OS cells, A549 cells, MCF-7 cells, 3T3 cells, HTB-9 cells, HeLa cells, HepG2 cells, HEKTE cells, SH-SY5Y cells, HUVEC cells, HMVEC cells, primary human fibroblasts, and primary human hepatocyte/3T3-J2 fibroblast co-cultures. In some embodiments a cell line used as a basis fora cell context is a culture of human cells. In some embodiments, a cell line used as a basis for a cell context is any cell line set forth in Table 1 below, or a genetic modification of such a cell line. In some embodiments each cell line used as a different cell context in the screening method is from the same species. In some embodiments the cell lines used for a cell context in the screening method can be from more than one species. For instance, a first cell line used as a first context is from a first species (e.g., human) and a second cell line used as a second context is from a second species (e.g., monkey).

TABLE 1
Example Cell Types Used as a Basis for Providing
Cell Context in Some Embodiments

| Cell Name | Tissue Type | Tissue | Phenotype | Primary |
|---|---|---|---|---|
| jb6 p+ c141 | Mouse | Skin | Adherent | no |
| jcam1.6 | Human | Lymphocyte | Suspension | no |
| jb6 rt101 | Mouse | Epithelial | Either | yes |
| jy | Human | Lymphocyte | Suspension | no |
| k562 | Human | Bone | Suspension | no |
| j82 | Human | Bladder | Adherent | no |
| ivec cells | Human | Endothelial | Adherent | no |
| jeg-3 | Human | Other | Adherent | no |
| jurkat | Human | Lymphocyte | Suspension | no |
| j558l | Mouse | Blood | Suspension | no |
| k46 | Mouse | Lymphocyte | Suspension | no |
| j774 cells | Mouse | Macrophage | Adherent | no |
| knrk | Rat | Epithelial | Either | no |
| keratinocytes | Mouse | Keratinocyte | Adherent | yes |
| kc1 | Drosophila | Default | Adherent | no |
| | Melanogaster | | | |
| kc18-2-40 cells | Human | Keratinocyte | Adherent | no |
| kt-3 | Human | Lymphocyte | Suspension | no |
| kmst-6 | Human | Skin | Adherent | no |
| l1210-fas | Mouse | Myoblast | Suspension | yes |
| kb | Human | Fibroblast | Adherent | no |
| keratinocytes | Human | Keratinocyte | Adherent | yes |
| kg-1 cells | Human | Bone marrow | Suspension | no |
| ks cells | Human | Skin | Adherent | yes |
| kd83 | Mouse | Blood | Suspension | no |
| l-m(tk-) | Mouse | Connective | Adherent | no |
| l8 cells | Rat | Myoblast | Adherent | yes |
| lk35.2 | Mouse | Lymphocyte | Suspension | no |
| l1210 | Mouse | Monocyte | Suspension | yes |
| lan-5 | Human | Brain | Adherent | no |
| llc-pk1 | Pig | Kidney | Adherent | no |
| lewis lung carcinoma, | Mouse | Lung | Either | no |
| llc | | | | |
| l6e9 | Rat | Muscle | Adherent | no |
| lmh | Chicken | Liver | Adherent | no |
| l6 cells | Rat | Muscle | Adherent | no |
| lisn c4 (nih 3t3 | Mouse | Fibroblast | Adherent | yes |
| derivative | | | | |
| overexpressing egf) | | | | |
| lap1 | Mouse | Lymphocyte | Suspension | yes |
| lap3 | Mouse | Embryo | Adherent | no |
| l929 | Mouse | Fibroblast | Adherent | no |
| mg87 | Mouse | Fibroblast | Adherent | no |
| min6 | Mouse | Default | Either | no |
| mel | Mouse | Other | Adherent | no |
| melenoma cells | Human | Melanoma | Adherent | yes |
| mdbk | Cow | Kidney | Adherent | no |
| mkn45 gastric cancer | Human | Stomach | Adherent | yes |
| mewo | Human | Melanoma | Adherent | no |
| mda-mb-468 | Human | Breast/Mammary | Adherent | no |
| mdck | Dog | Kidney | Adherent | no |
| mf4/4 | Mouse | Macrophage | Adherent | no |
| me-180 | Human | Cervix | Adherent | yes |
| mes-sa | Human | Uterus | Adherent | no |
| mg-63 cells | Human | Bone | Adherent | no |
| mono-mac-6 cells | Human | Blood | Suspension | no |
| monocytes | Human | Blood | Suspension | yes |
| mrc-5 | Human | Lung | Adherent | yes |
| mob cells | Mouse | Osteoblast | Adherent | yes |
| msc human | Human | Bone marrow | Adherent | yes |
| mesenchymal stem cell | | | | |
| mt-2 | Human | Lymphocyte | Adherent | yes |
| mouse embryonic | Mouse | Fibroblast | Adherent | yes |
| fibroblasts | | | | |
| mnt1 | Human | Skin | Adherent | yes |
| ms1 | Mouse | Pancreas | Adherent | no |
| mr1 | Rat | Embryo | Adherent | no |
| mt4 | Human | Lymphocyte | Suspension | yes |
| molt4 (human acute t | Human | Blood | Suspension | no |
| lymphoblastic | | | | |
| leukaemia) | | | | |
| hep3b | Human | Liver | Adherent | no |
| hepatic stellate cells | Rat | Liver | Adherent | yes |
| hela 229 cells | Human | Cervix | Either | yes |
| hep2 | Human | Epithelial | Adherent | no |
| hela-cd4 | Human | Epithelial | Adherent | no |
| hct116 | Human | Colon | Adherent | no |
| hepatocytes | Mouse | Liver | Adherent | yes |
| hela s3 | Human | Cervix | Adherent | no |
| hel | Human | Lymphocyte | Suspension | yes |
| hela cells | Human | Cervix | Adherent | no |
| hela t4 | Human | Blood | Suspension | no |
| hepg2 | Human | Liver | Adherent | no |
| high 5 (bti-tn-5b1-4) | Insect | Embryo | Adherent | no |
| hit-t15 cells | Hamster | Epithelial | Adherent | no |
| hepatocytes | Rat | Liver | Adherent | yes |
| hitb5 | Human | Muscle | Adherent | yes |
| hi299 | Human | Lung | Adherent | no |
| hfff2 | Human | Foreskin | Adherent | yes |
| hib5 | Rat | Brain | Adherent | yes |
| hm-1 embryonic stem | Mouse | Other | Adherent | yes |
| cells | | | | |
| hitb5 | Human | Muscle | Adherent | yes |
| hl-60 | Human | Lymphocyte | Suspension | no |
| hl-5 | Mouse | Heart | Adherent | no |
| hl-1 | Mouse | Heart | Adherent | no |
| glya | Hamster | Ovary | Adherent | no |
| gamma 3t3 | Mouse | Fibroblast | Adherent | no |
| gh3 | Rat | Pituitary | Adherent | no |
| granta-519 | Human | Blood | Suspension | no |
| freestyle 293 | Human | Kidney | Suspension | no |
| g401 | Human | Connective | Adherent | no |
| fto-2b (rat hepatoma) | Rat | Liver | Suspension | yes |
| cells | | | | |
| gh4c1 | Rat | Pituitary | Adherent | yes |
| fsdc, murine dendritic | Mouse | Blood | Either | no |
| cell | | | | |
| goto | Human | Neuroblastoma | Adherent | yes |
| gc-2spd (ts) | Mouse | Epithelial | Adherent | no |
| glomeruli | Rat | Lung | Adherent | yes |
| frt | Rat | Thyroid | Suspension | no |
| h19-7/igf-ir | Rat | Brain | Suspension | no |
| gt1 | Mouse | Brain | Adherent | no |
| griptite? 293 msr | Human | Kidney | Adherent | no |
| h441 | Human | Lung | Adherent | yes |
| h-500, leydig tumor cell | Rat | Testes | Adherent | yes |
| h4 | Human | Glial | Adherent | no |
| guinea pig endometrial | Guinea Pig | Ovary | Adherent | yes |
| stromal cells | | | | |
| h187 | Human | Lung | Adherent | yes |
| h35 | Rat | Liver | Adherent | no |
| h-7 | Mouse | Bone marrow | Suspension | no |
| h1299 | Human | Lung | Adherent | no |
| granulosa cells | Mouse | Ovary | Either | yes |
| hbl100 cells | Human | Breast/Mammary | Adherent | no |
| h9c2 | Rat | Myoblast | Adherent | no |
| hbec-90 | Human | Brain | Adherent | no |
| has-p | Mouse | Breast/Mammary | Adherent | yes |
| hasmcs | Human | Muscle | Adherent | no |
| hc11 | Mouse | Breast/Mammary | Adherent | no |
| hacat | Human | Keratinocyte | Adherent | yes |
| hb60-5 cells | Mouse | Spleen | Adherent | no |
| h4iie | Rat | Liver | Adherent | yes |
| hca-7 | Human | Colon | Adherent | yes |
| hcd57 | Mouse | Blood | Suspension | no |
| haecs | Human | Aorta | Adherent | yes |
| rpe.40 | Hamster | Kidney | Adherent | yes |
| rcme, rabbit coronary | Rabbit | Endothelial | Adherent | yes |
| microvessel endothelial | | | | |
| rko, rectal carcinoma | Human | Colon | Adherent | no |
| cell line | | | | |
| ros, rat osteoblastic cell | Rat | Osteoblast | Adherent | yes |
| line | | | | |
| rh18 | Human | Muscle | Adherent | no |
| rcho | Rat | Default | Adherent | no |
| rccd1 | Rat | Kidney | Adherent | no |
| s194 cells | Mouse | Lymphocyte | Adherent | yes |
| rin 1046-38 | Rat | Pancreas | Suspension | no |
| rw-4 | Mouse | Embryo | Adherent | yes |
| rj2.2.5 | Human | Lymphocyte | Suspension | no |
| rk13 | Rabbit | Kidney | Adherent | no |
| remc | Rat | Breast/Mammary | Adherent | no |
| sk-br-3 | Human | Breast/Mammary | Adherent | no |
| s49.1 | Mouse | Thymus | Suspension | no |
| schizosaccharomyces | Yeast | Other | Either | yes |
| pombe | | | | |
| sf9 | Insect | Ovary | Suspension | no |
| sf21 | Insect | Other | Either | yes |
| sf21ae | Insect | Other | Either | yes |
| sh-sy5y | Human | Brain | Either | no |
| s2-013 | Human | Pancreas | Either | yes |
| saos-2 | Human | Bone | Adherent | no |
| siha | Human | Cervix | Adherent | no |
| scc12, human squamous | Human | Skin | Adherent | yes |
| cell carcinoma line | | | | |
| (c12c20) | | | | |
| shep | Human | Brain | Adherent | no |
| sk-lms-1 | Human | Other | Adherent | no |
| sk-n-sh, neuronal cells | Human | Brain | Adherent | yes |
| sk-n-as | Human | Neuroblastoma | Adherent | no |
| sknmc | Human | Brain | Adherent | no |
| sk-hep-1 cells | Human | Skin | Either | yes |
| skov3 | Human | Ovary | Adherent | no |
| sk-n-be(2) | Human | Neuroblastoma | Adherent | yes |
| smmc7721 | Human | Liver | Adherent | no |
| smooth muscle cells | Rat | Aorta | Adherent | yes |
| (aortic) rasmc (a7-r5) | | | | |
| sl2 | Drosophila | Default | Either | no |
| | melanogaster | | | |
| sk-ut-1 | Human | Muscle | Adherent | no |
| n2a | Mouse | Neuroblastoma | Adherent | no |
| myocytes (ventricular) | Rat | Heart | Adherent | yes |
| mtln3 | Rat | Breast/Mammary | Adherent | no |
| n1e-115 | Mouse | Brain | Adherent | no |
| mtsv1-7 | Human | Epithelial | Adherent | no |
| murine alveolar | Rat | Lung | Adherent | no |
| macrophages cell line | | | | |
| mhs | | | | |
| n18tg cells | Mouse | Neuroblastoma | Adherent | no |
| n13 | Mouse | Brain | Adherent | no |
| mutu group3, b-cell line | Human | Lymphocyte | Suspension | no |
| mtd-1a | Mouse | Epithelial | Adherent | yes |
| mutu i | Human | Lymphocyte | Suspension | no |
| mv1lu | Mink | Lung | Adherent | no |
| ncb20 | Mouse | Neuroblastoma | Adherent | yes |
| nb324k | Human | Kidney | Adherent | no |
| neural stem cells | Rat | Brain | Either | yes |
| neuroblastoma | Human | Brain | Adherent | yes |
| nci-h23 | Human | Lung | Adherent | no |
| nci-h460 | Human | Lung | Adherent | no |
| neurons (astrocytes) | Rat | Brain | Adherent | yes |
| neuro 2a, a murine | Mouse | Neuroblastoma | Adherent | no |
| neuroblastoma cell line | | | | |
| nbt-ii | Rat | Bladder | Adherent | no |
| neuons (astrocytes) | Rat | Astrocyte | Adherent | yes |
| nci-h295 | Human | Kidney | Adherent | no |
| nci-h358 | Human | Lung | Adherent | no |
| neuons (hippocampal & | Rat | Brain | Adherent | yes |
| septal) | | | | |
| neurons | Mouse | Brain | Adherent | yes |
| nhdf | Human | Fibroblast | Adherent | no |
| neurons (post- | Rat | Brain | Adherent | yes |
| natal/adult) | | | | |
| nhbe | Human | Lung | Adherent | yes |
| ng108-15 | Mouse | Neuroblastoma | Adherent | no |
| neurons (embryonic | Rat | Brain | Adherent | yes |
| cortical) | | | | |
| neurons (cortical) | Mouse | Other | Adherent | yes |
| ng 125 | Human | Neuroblastoma | Adherent | no |
| nhf3 | Human | Fibroblast | Adherent | no |
| neurospora crassa | Fungi | Embryo | Adherent | yes |
| neurons (superior | Rat | Brain | Adherent | yes |
| cervical ganglia - scg) | | | | |
| neurons (ganglia) | Frog | Brain | Either | yes |
| ns20y | Mouse | Neuroblastoma | Adherent | no |
| nrk | Rat | Fibroblast | Adherent | yes |
| nmumg | Mouse | Breast/Mammary | Adherent | no |
| o23 | Hamster | Fibroblast | Adherent | no |
| nt2 | Human | Fibroblast | Adherent | no |
| nhff | Human | Foreskin | Adherent | yes |
| nih 3t3, 3t3-l1 | Mouse | Fibroblast | Adherent | no |
| ohio helas | Human | Cervix | Suspension | no |
| nih 3t6 | Mouse | Fibroblast | Adherent | no |
| nih 3t3-l1, nih 3t3 | Mouse | Embryo | Adherent | no |
| nt2-d1 | Human | Testes | Adherent | no |
| nih 3t3-l1, nih 3t3 ( ) | Mouse | Embryo | Adherent | no |
| orbital fibroblast | Human | Fibroblast | Adherent | yes |
| osteoblasts | Rat | Bone | Adherent | yes |
| p19 cells | Mouse | Embryo | Adherent | yes |
| ovcar-3 | Human | Ovary | Adherent | no |
| opaec cells | Sheep | Endothelial | Adherent | no |
| ovarian surface | Human | Ovary | Adherent | yes |
| epithelial (ose) | | | | |
| p388d1 | Mouse | Macrophage | Adherent | yes |
| p825, mastocytoma cells | Mouse | Macrophage | Adherent | yes |
| p19cl6 | Mouse | Heart | Adherent | no |
| omega e | Mouse | Embryo | Adherent | no |
| ok, derived from renal | Opossum | Kidney | Adherent | yes |
| proximal tubules | | | | |
| p815, mastocytoma cells | Mouse | Macrophage | Adherent | yes |
| p3.653 × ag8 murine | Mouse | Bone marrow | Adherent | yes |
| myeloma cells | | | | |
| paju, human neural | Human | Brain | Adherent | yes |
| crest-derived cell line | | | | |
| pac-1 | Rat | Aorta | Adherent | no |
| parp−/− mouse | Mouse | Fibroblast | Suspension | no |
| embryonic fibroblasts | | | | |
| pci-13 | Human | Skin | Adherent | no |
| pc 6 | Rat | Glial | Adherent | no |
| (pheochromocytoma-6) | | | | |
| pancreatic islets | Rat | Pancreas | Adherent | yes |
| peripheral blood | Human | Blood | Either | yes |
| lymphocytes | | | | |
| pc-3 | Human | Prostate | Either | no |
| pc-12 | Rat | Brain | Adherent | no |
| panc1 | Human | Pancreas | Adherent | no |
| per.c6 ® | Human | Retina | Either | no |
| pa 317 or pt67 mouse | Mouse | Fibroblast | Adherent | yes |
| fibroblast with herpes | | | | |
| thymidine kinase (tk) | | | | |
| gene | | | | |
| pam212, mouse | Mouse | Keratinocyte | Adherent | yes |
| keratinocytes | | | | |
| peripheral blood | Human | Blood | Suspension | yes |
| mononuclear cells | | | | |
| (pbmc) | | | | |
| qt6 | Quail | Fibroblast | Adherent | no |
| pu5-1.8 cells | Mouse | Macrophage | Suspension | no |
| primary lymphoid (oka) | Shrimp | Lymphocyte | Adherent | yes |
| organ from penaeus | | | | |
| shrimp | | | | |
| ps120, an nhe-deficient | Hamster | Lung | Adherent | yes |
| clone derived from | | | | |
| ccl39 cells | | | | |
| phoenix-eco cells | Human | Embryo | Adherent | no |
| quail embryos | Quail | Embryo | Either | yes |
| plb985 | Human | Blood | Suspension | no |
| rabbit pleural | Rabbit | Lung | Adherent | no |
| mesothelial | | | | |
| r1 embryonic stem cell, | Mouse | Embryo | Either | no |
| es | | | | |
| rabbit vsmc, vascular | Rabbit | Muscle | Adherent | yes |
| smooth muscle cells | | | | |
| raec, rat aortic | Rat | Aorta | Adherent | yes |
| endothelial cells | | | | |
| raji | Human | Lymphocyte | Suspension | no |
| rat epithelial cells | Rat | Epithelial | Adherent | yes |
| raw 264.7 cells, murine | Mouse | Macrophage | Adherent | yes |
| macrophage cells | | | | |
| ramos | Human | Lymphocyte | Suspension | no |
| rat hepatic ito cells | Rat | Liver | Adherent | yes |
| rat adipocyte | Rat | Adipose | Adherent | yes |
| rat c5, glioma cells | Rat | Glial | Adherent | yes |
| rat-1, rat fibroblasts | Rat | Fibroblast | Adherent | yes |
| rat 2, rat fibroblasts | Rat | Fibroblast | Adherent | yes |
| rat glomerular mesangial | Rat | Kidney | Adherent | yes |
| mc cells | | | | |
| raw cells | Rat | Peritoneum | Suspension | no |
| rat-6 (r6), rat embryo | Rat | Fibroblast | Adherent | yes |
| fibroblast | | | | |
| hmec-1 | Human | Endothelial | Adherent | yes |
| hre h9 | Rabbit | Uterus | Adherent | no |
| hmn 1 | Mouse | Neuroblastoma | Adherent | yes |
| ht-29 | Human | Colon | Adherent | no |
| hos | Human | Osteoblast | Adherent | no |
| hs68 | Human | Foreskin | Adherent | yes |
| hmcb | Human | Skin | Adherent | no |
| hs-578t | Human | Breast/Mammary | Adherent | no |
| hnscc | Human | Skin | Adherent | no |
| hpb-all | Human | Lymphocyte | Suspension | no |
| hmvec-l | Human | Lung | Adherent | no |
| hsy-eb | Human | Other | Adherent | no |
| huh 7 | Human | Liver | Adherent | no |
| htlm2 | Mouse | Breast/Mammary | Adherent | yes |
| hut 78 | Human | Skin | Suspension | no |
| ht1080 | Human | Fibroblast | Adherent | no |
| huvec, huaec | Human | Umbilicus | Adherent | yes |
| htla230 | Human | Neuroblastoma | Adherent | yes |
| hybridoma | Mouse | Spleen | Suspension | no |
| ib3-1 | Human | Lung | Adherent | no |
| ht22 | Mouse | Brain | Adherent | yes |
| human skeletal muscle | Human | Muscle | Adherent | yes |
| ht4 | Human | Testes | Adherent | yes |
| hutu 80 | Human | Colon | Adherent | yes |
| in vivo mouse brain | Mouse | Bone | Either | yes |
| in vivo rat brain | Rat | Brain | Either | yes |
| iec-6 rie | Rat | Epithelial | Adherent | no |
| imr-32 | Human | Neuroblastoma | Adherent | no |
| ic11 | Mouse | Testes | Adherent | no |
| imr-90 | Human | Lung | Adherent | no |
| in vivo rat lung | Rat | Lung | Either | yes |
| in vivo rat liver | Rat | Liver | Either | yes |
| ins-1 | Rat | Pancreas | Adherent | no |
| in vivo rabbit eye | Rabbit | Other | Either | yes |
| in vivo mouse | Mouse | Other | Either | yes |
| imdf | Mouse | Skin | Adherent | no |
| in vivo pig | Pig | Other | Either | yes |
| caski | Human | Cervix | Adherent | no |
| cerebellar | Mouse | Brain | Adherent | yes |
| cd34+ monocytes | Human | Monocyte | Suspension | yes |
| cfk2 | Rat | Bone | Adherent | no |
| cem | Human | Blood | Suspension | no |
| catha, cath.a | Mouse | Brain | Either | no |
| ccl-16-b9 | Hamster | Lung | Adherent | no |
| ch12f3-2a | Mouse | Lymphocyte | Suspension | no |
| cf2th | Dog | Thymus | Adherent | no |
| cardiomyocytes | Human | Heart | Adherent | yes |
| cg-4 | Rat | Glial | Adherent | no |
| cell.220(b8) | Human | Default | Suspension | no |
| cardiomyocytes | Rat | Heart | Adherent | yes |
| chick embryo fibroblasts | Chicken | Embryo | Adherent | yes |
| chicken sperm | Chicken | Sperm | Adherent | yes |
| cho k1 | Hamster | Ovary | Adherent | no |
| cho 58 | Hamster | Ovary | Adherent | no |
| cho-b7 | Hamster | Ovary | Adherent | no |
| chick embryo | Chicken | Embryo | Adherent | yes |
| blastodermal cells | | | | |
| cho -b53 | Hamster | Ovary | Adherent | yes |
| chick embryo | Chicken | Embryo | Adherent | yes |
| chondrocytes | | | | |
| chinese hamster lung | Hamster | Lung | Adherent | no |
| cho dg44 | Hamster | Ovary | Either | no |
| cho - b53 jf7 | Hamster | Ovary | Adherent | yes |
| chicken hepatocytes | Chicken | Liver | Adherent | yes |
| cos-1 | Primate - Non | Kidney | Adherent | no |
| | Human | | | |
| cho-lec1 | Hamster | Ovary | Adherent | yes |
| clone a | Human | Colon | Adherent | no |
| cho-lec2 | Hamster | Ovary | Adherent | no |
| colo205 | Human | Colon | Adherent | no |
| chu-2 | Human | Epithelial | Adherent | no |
| cmt-93 | Mouse | Rectum | Adherent | no |
| cho-s | Hamster | Ovary | Suspension | no |
| cho-leu c2gnt | Hamster | Ovary | Adherent | no |
| cho-trvb | Hamster | Ovary | Adherent | no |
| clone-13, mutant b | Human | Lymphocyte | Suspension | no |
| lymphoblastoid | | | | |
| cj7 | Mouse | Embryo | Adherent | no |
| smooth muscle cells | Rat | Muscle | Adherent | yes |
| (aortic) | | | | |
| splenocytes | Mouse | Spleen | Suspension | yes |
| smooth muscle cells | Rat | Muscle | Adherent | yes |
| (vascular) | | | | |
| sp1 | Mouse | Breast/Mammary | Adherent | no |
| stem | Rat | Bone | Suspension | yes |
| spoc-1 | Rat | Trachael | Adherent | no |
| snb19 | Human | Brain | Adherent | no |
| splenocytes (resting b | Mouse | Spleen | Suspension | yes |
| cells) | | | | |
| splenocytes (b cells t2) | Mouse | Spleen | Suspension | yes |
| svr | Mouse | Pancreas | Adherent | no |
| stem cells | Human | Bone marrow | Suspension | yes |
| smooth muscle cells | Human | Muscle | Adherent | yes |
| (vascular) | | | | |
| smooth muscle cells | Rabbit | Aorta | Adherent | yes |
| (vascular) | | | | |
| t3cho/at1a | Hamster | Ovary | Either | no |
| t-rex-cho | Hamster | Ovary | Adherent | no |
| t-rex-293 | Human | Kidney | Adherent | no |
| sw620 | Human | Colon | Adherent | no |
| t lymphocytes (t cells) | Mouse | Lymphocyte | Adherent | yes |
| t lymphocytes cytotoxic | Mouse | Lymphocyte | Either | yes |
| (ctl) cells | | | | |
| sw480 | Human | Colon | Adherent | no |
| t lymphocytes (t cells) | Human | Lymphocyte | Adherent | yes |
| sw13 | Human | Adrenal | Adherent | no |
| | | gland/cortex | | |
| t47d, t-47d | Human | Breast/Mammary | Adherent | no |
| t24 | Human | Bladder | Adherent | no |
| t-rex hela | Human | Cervix | Adherent | no |
| tr2 | Mouse | Brain | Adherent | no |
| tig | Human | Fibroblast | Adherent | yes |
| t98g | Human | Brain | Adherent | no |
| tsa201 | Human | Embryo | Adherent | no |
| tobacco protoplasts | Plant | Other | Suspension | yes |
| thp-1 | Human | Blood | Suspension | yes |
| tk.1 | Mouse | Lymphocyte | Suspension | no |
| tib-90 | Mouse | Fibroblast | Adherent | no |
| ta3 | Mouse | Breast/Mammary | Adherent | no |
| tyknu cells | Human | Ovary | Adherent | no |
| u-937 | Human | Macrophage | Suspension | no |
| tgw-nu-1 | Human | Bladder | Adherent | no |
| b-lcl | Human | Blood | Suspension | no |
| b4.14 | Primate - Non | Kidney | Adherent | yes |
| | Human | | | |
| b82 m721 | Mouse | Fibroblast | Adherent | no |
| b-tc3 | Mouse | Pancreas | Adherent | no |
| b16-f10 | Mouse | Melanoma | Adherent | no |
| b82 | Mouse | Fibroblast | Adherent | no |
| as52 | Hamster | Ovary | Adherent | no |
| b lymphocytes | Human | Blood | Suspension | yes |
| b35 | Rat | Neuroblastoma | Adherent | yes |
| b65 | Rat | Neuroblastoma | Adherent | no |
| b11 | Mouse | Spleen | Suspension | no |
| att-20 | Mouse | Pituitary | Adherent | no |
| bcl-1 | Mouse | Lymphocyte | Adherent | no |
| bac | Cow | Adrenal Gland | Adherent | yes |
| balb/c 3t3, 3t3-a31 | Mouse | Fibroblast | Adherent | no |
| be(2)-c | Human | Neuroblastoma | Adherent | no |
| bewo | Human | Other | Adherent | no |
| balb/mk | Mouse | Epithelial | Adherent | no |
| beas-2b | Human | Lung | Adherent | no |
| bewo | Human | Uterus | Adherent | yes |
| baf3, ba/fi | Mouse | Lymphocyte | Suspension | no |
| bcec | Human | Brain | Adherent | yes |
| bc3h1 | Mouse | Brain | Adherent | yes |
| baec | Cow | Aorta | Adherent | no |
| a10 | Rat | Muscle | Adherent | no |
| a1.1 | Mouse | Lymphocyte | Adherent | yes |
| a72 | Dog | Connective | Adherent | no |
| a549 | Human | Lung | Adherent | no |
| a204 | Human | Muscle | Adherent | yes |
| a6 | Frog | Kidney | Adherent | no |
| a875 | Human | Melanoma | Adherent | yes |
| a498 | Human | Kidney | Adherent | no |
| a172 | Human | Brain | Adherent | yes |
| a-431 | Human | Skin | Adherent | no |
| a20 | Mouse | Lymphocyte | Suspension | yes |
| arpe-19 | Human | Retina | Adherent | no |
| alpha t3 | Human | Pituitary | Adherent | no |
| akr | Mouse | Spleen | Adherent | no |
| ar4-2j | Rat | Pancreas | Adherent | no |
| aortic endothelial cells | Human | Aorta | Adherent | yes |
| achn | Human | Kidney | Adherent | yes |
| adventitial fibroblasts | Human | Aorta | Adherent | yes |
| am12 | Mouse | Blood | Suspension | no |
| anterior pituitary | Human | Pituitary | Adherent | yes |
| gonadotropes | | | | |
| ae-1 | Mouse | Spleen | Suspension | no |
| ab1 | Mouse | Embryo | Adherent | no |
| anjou 65 | Human | Default | Either | no |
| crfk | Cat | Kidney | Adherent | no |
| d.mel-2 | Insect | Embryo | Either | no |
| ct26 | Mouse | Colon | Either | yes |
| cowpea plant embryos | Fungi | Embryo | Adherent | yes |
| cos-7 | Primate - Non | Kidney | Adherent | no |
| | Human | | | |
| crl6467 | Mouse | Liver | Adherent | no |
| cwr22rv1 | Human | Prostate | Adherent | no |
| ct60 | Hamster | Ovary | Adherent | no |
| cos-gs1 | Primate - Non | Kidney | Adherent | no |
| | Human | | | |
| cos-m6 | Primate - Non | Kidney | Adherent | yes |
| | Human | | | |
| cv-1 | Primate - Non | Kidney | Adherent | no |
| | Human | | | |
| ctll-2 | Mouse | Lymphocyte | Suspension | no |
| d3 embryonic stem cells | Mouse | Embryo | Adherent | no |
| du145 | Human | Prostate | Adherent | no |
| do-11.10 | Mouse | Lymphocyte | Suspension | no |
| daudi | Human | Lymphocyte | Suspension | no |
| d10 | Mouse | Lymphocyte | Suspension | no |
| dgz | Plant | Other | Adherent | yes |
| dictyostelium | Amoeba | Other | Suspension | yes |
| dt40 | Chicken | Bursa | Suspension | no |
| drosophila kc | Insect | Embryo | Adherent | yes |
| df1 | Chicken | Fibroblast | Adherent | no |
| dc 2.4 cells | Mouse | Blood | Either | no |
| daoy | Human | Other | Adherent | no |
| lovo | Human | Colon | Adherent | no |
| lncap | Human | Prostate | Adherent | no |
| m21 | Human | Melanoma | Adherent | no |
| lsv5 | Human | Keratinocyte | Adherent | no |
| ltk | Mouse | Connective | Adherent | no |
| m1 | Rat | Embryo | Adherent | no |
| m3z | Human | Breast/Mammary | Adherent | no |
| m21-l | Human | Melanoma | Adherent | no |
| lymphoid cell line | Rat | Lymphocyte | Suspension | no |
| m-imcd | Mouse | Kidney | Adherent | yes |
| m12.4 | Mouse | Lymphocyte | Adherent | no |
| m21-14 | Human | Melanoma | Adherent | no |
| mat b iii | Rat | Breast/Mammary | Adherent | no |
| mda-mb-453 | Human | Breast/Mammary | Adherent | no |
| mca-rh7777 | Rat | Liver | Adherent | no |
| ma104 | Primate - Non | Kidney | Adherent | no |
| | Human | | | |
| magi-ccr5 | Human | Epithelial | Adherent | no |
| mda-mb-231 | Human | Breast/Mammary | Adherent | no |
| mcf-10 | Human | Breast/Mammary | Adherent | no |
| mc3t3-e1 | Mouse | Osteoblast | Adherent | no |
| mc ardle 7777 | Rat | Liver | Either | yes |
| macrophages | Mouse | Peritoneum | Adherent | yes |
| mcf-7 | Human | Breast/Mammary | Adherent | no |
| macrophages | Human | Blood | Either | yes |
| maize protoplasts | Plant | Other | Adherent | no |
| umr 106-01 | Rat | Bone | Adherent | no |
| uc729-6 | Human | Lymphocyte | Either | no |
| u9737 | Human | Lymphocyte | Suspension | no |
| uok257 | Human | Kidney | Adherent | no |
| u373mg | Human | Astrocyte | Adherent | no |
| wit49 wilms tumor | Human | Lung | Either | yes |
| vero | Primate - Non | Kidney | Adherent | no |
| | Human | | | |
| u87, u87mg | Human | Astrocyte | Adherent | no |
| umrc6 | Human | Kidney | Adherent | no |
| u251 cells | Human | Glial | Adherent | no |
| u2os | Human | Bone | Adherent | no |
| bovine chromaffin cells | Cow | Adrenal Gland | Adherent | yes |
| bowes melanoma cells | Human | Skin | Adherent | no |
| boll weevil brl-ag-3c | Insect | Other | Adherent | no |
| bm5 | Insect | Ovary | Suspension | no |
| bhk-21 | Hamster | Kidney | Either | no |
| bosc 23 | Human | Kidney | Adherent | yes |
| bms-black mexican | Default | Default | Suspension | yes |
| sweet protoplasts | | | | |
| bfc012 | Mouse | Embryo | Adherent | no |
| bone marrow cells | Mouse | Bone marrow | Suspension | yes |
| bone marrow derived | Human | Bone marrow | Adherent | yes |
| stromal cells | | | | |
| bs-c-1, bsc-1 | Primate - Non | Kidney | Adherent | no |
| | Human | | | |
| bjab | Human | Lymphocyte | Suspension | no |
| bnl cl.2 (cl2) | Mouse | Liver | Adherent | no |
| btm (bovine trachael | Cow | Muscle | Adherent | no |
| myocytes) | | | | |
| c2c12 | Mouse | Muscle | Adherent | no |
| c3a | Human | Liver | Adherent | no |
| c1.39t | Human | Fibroblast | Adherent | no |
| bt cells | Cow | Fibroblast | Adherent | no |
| bsc-40 | Primate - Non | Kidney | Adherent | no |
| | Human | | | |
| c33 | Human | Cervix | Adherent | no |
| c1c12 | Mouse | Muscle | Adherent | no |
| c127 | Mouse | Epithelial | Adherent | no |
| bt549 | Human | Breast/Mammary | Adherent | no |
| c1r, hmy2.c1r | Human | Lymphocyte | Adherent | yes |
| c13-nj | Human | Glial | Adherent | no |
| canine gastric parietal | Dog | Stomach | Adherent | yes |
| cells | | | | |
| calu-3 | Human | Lung | Adherent | yes |
| cak | Mouse | Fibroblast | Adherent | no |
| c57bl/6 cells | Mouse | Heart | Adherent | no |
| caco-2 cells | Human | Colon | Adherent | no |
| c3h 10t1/2 | Mouse | Fibroblast | Adherent | no |
| ca77 | Rat | Thyroid | Adherent | no |
| c6 cells | Rat | Brain | Adherent | no |
| calu-6 | Human | Lung | Adherent | no |
| capan-2 | Human | Pancreas | Adherent | no |
| c4-2 | Human | Prostate | Adherent | no |
| 143b | Human | Bone marrow | Either | no |
| 1064sk | Human | Foreskin | Adherent | yes |
| 16-9 | Human hamster | Other | Adherent | no |
| | hybrid cell line - | | | |
| | transfected with | | | |
| | two human genes | | | |
| 2008 | Human | Ovary | Adherent | no |
| 208f | Rat | Fibroblast | Adherent | no |
| 293-h | Human | Kidney | Either | no |
| 293 | Human | Kidney | Either | no |
| 293 ebna | Human | Kidney | Adherent | no |
| 293t | Human | Kidney | Either | no |
| 2pk3 | Mouse | Lymphocyte | Suspension | no |
| 293-f | Human | Kidney | Either | no |
| 2780 | Human | Ovary | Adherent | no |
| 293s | Human | Kidney | Either | no |
| 2774 | Human | Ovary | Adherent | no |
| 3y1 | Rat | Fibroblast | Adherent | yes |
| 82-6 | Human | Fibroblast | Adherent | no |
| 9hte | Human | Trachael | Adherent | yes |
| 3.12 | Mouse | Lymphocyte | Either | yes |
| 5637 | Human | Bladder | Adherent | no |
| 4t1 | Mouse | Breast/Mammary | Adherent | no |
| 3t3-f442a | Mouse | Other | Adherent | yes |
| 33.1.1 | Mouse | Lymphocyte | Suspension | no |
| 32d | Mouse | Bone marrow | Either | no |
| 4de4 | Mouse | Bone marrow | Either | yes |
| e1-ts20 | Human | Breast/Mammary | Adherent | yes |
| embryonic stem cells | Mouse | Embryo | Adherent | yes |
| e. histolytica | Amoeba | Other | Suspension | yes |
| ef88 | Mouse | Fibroblast | Adherent | yes |
| el-4 | Mouse | Thymus | Suspension | no |
| ebc-1 | Human | Lung | Adherent | no |
| duck (in vivo) | Duck | Other | Suspension | yes |
| ecv | Human | Endothelial | Adherent | no |
| ecr-293 | Human | Kidney | Adherent | no |
| e14tg2a | Mouse | Embryo | Adherent | no |
| e36 | Hamster | Lung | Adherent | no |
| endothelial cells | Rat | Aorta | Adherent | yes |
| (pulmonary aorta) | | | | |
| endothelial cells (aortic) | Pig | Aorta | Adherent | yes |
| ewing sarcoma coh cells | Human | Bone | Suspension | no |
| f9 | Mouse | Testes | Adherent | no |
| fibroblasts (cardiac) | Rat | Fibroblast | Adherent | yes |
| f442-a | Mouse | Preadiopocyte | Adherent | no |
| es-2 ovarian clear cell | Human | Ovary | Adherent | no |
| adenocarcinoma | | | | |
| fetal neurons | Rat | Brain | Adherent | yes |
| epithelial cells | Human | Epithelial | Adherent | yes |
| (sra01/04) | | | | |
| fibroblasts (embryo) | Rat | Fibroblast | Adherent | yes |
| fgc-4 | Rat | Liver | Adherent | yes |
| fak−/− | Mouse | Embryo | Adherent | yes |
| es-d3 | Mouse | Embryo | Adherent | no |
| epithelial cells (rte) | Rat | Trachael | Adherent | yes |
| foreskin fibroblast | Human | Foreskin | Adherent | no |
| flp-in jurkat | Human | Lymphocyte | Suspension | no |
| flp-in cho | Hamster | Ovary | Adherent | no |
| fibroblasts (neonatal | Human | Skin | Adherent | yes |
| dermal) | | | | |
| flp-in 293 | Human | Kidney | Adherent | no |
| flp-in t-rex 293 | Human | Kidney | Adherent | no |
| flp-in cv-1 | Primate - Non | Kidney | Adherent | no |
| | Human | | | |
| fibroblasts | Chicken | Skin | Adherent | yes |
| fibroblasts (normal) | Human | Fibroblast | Adherent | yes |
| fl5.12 | Mouse | Liver | Suspension | no |
| fm3a | Mouse | Breast/Mammary | Adherent | no |
| fr | Rat | Fibroblast | Adherent | no |
| nalm6 | Human | Other | Suspension | no |

As described above, in some embodiments, in the test states and control states the cell context is further perturbed, e.g., to simulate a disease phenotype. In some embodiments, the perturbation is an environmental factor applied to the cell context, e.g., that perturbs the cell relative to a reference environment (such as a growth medium that is commonly used to culture the particular cell). For example, in some embodiments, the cell context includes a component in a growth medium that significantly changes the metabolism of the one or more cells, e.g., a compound that is toxic to the one or more cells, that slows cellular metabolism, that increases cellular metabolism, that inhibits a checkpoint, that disrupts mitosis and/or meiosis, or that otherwise changes a characteristic of cellular metabolism. As other examples, the perturbation could be a shift in the osmolality, conductivity, pH, or other physical characteristic of the growth environment.

In some embodiments, the perturbation includes a mutation within the genome of the one or more cells, e.g., a human cell line in which a gene has been mutated or deleted. In some embodiments, a cell context is a cell line that has one or more documented structural variations (e.g., a documented single nucleotide polymorphism “SNP”, an inversion, a deletion, an insertion, or any combination thereof). In some such embodiments, the one or more documented structural variations are homozygous variations. In some such embodiments, the one or more documented structural variations are heterozygous variations. As an example of a homozygous variation in a diploid genome, in the case of a SNP, both chromosomes contain the same allele for the SNP. As an example of a heterozygous variation in a diploid genome, in the case of the SNP, one chromosome has a first allele for the SNP and the complementary chromosome has a second allele for the SNP, where the first and second allele are different.

In some embodiments, the perturbation includes one or more nucleic acid (e.g., one or more siRNA) that are designed to suppress (e.g., knock-down or knock-out) expression of one or more genes in one or more cell types of the cell context. In some embodiments, the perturbation includes a plurality of nucleic acids (e.g., a plurality of siRNA) that are designed to suppress expression of the same gene in one or more cell types of the cell context. For example, 2, 3, 4, 5, 6, 7, 8, 9, 10, or more siRNA molecules targeting different sequences (e.g., overlapping and/or non-overlapping) of the same gene. In some embodiments, the perturbation includes one or more nucleic acid (e.g., one or more siRNA) that are designed to suppress expression of multiple genes, e.g., 2, 3, 4, 5, 6, 7, 8, 9, 10, or more genes. In some embodiments, the plurality of genes express proteins involved in a common pathway (e.g., a metabolic or signaling pathway) in one or more cell types of the cell context. In some embodiments, the plurality of genes express proteins involved in different pathways in one or more cell types of the cell context. In some embodiments, the different pathways are partially redundant pathways for a particular biological function, e.g., different cell cycle checkpoint pathways. In some embodiments, the perturbation suppresses expression of a gene known to be associated with a disease (e.g., a checkpoint inhibitor gene associated with a cancer). In some embodiments, the perturbation suppresses expression of a gene known to be associated with a cellular phenotype (e.g., a gene that causes a metabolic phenotype in cultured cells when suppressed). In some embodiments, the perturbation suppresses expression of a gene that has not previously been associated with a disease or cellular phenotype.

In some embodiments, a cell context is perturbed by exposure to a small interfering RNA (siRNA), e.g., a double-stranded RNA molecule, 20-25 base pairs in length that interferes with the expression of a specific gene with a complementary nucleotide sequence by degrading mRNA after transcription preventing translation of the gene. An siRNA is an RNA duplex that can reduce gene expression through enzymatic cleavage of a target mRNA mediated by the RNA induced silencing complex (RISC). An siRNA has the ability to inhibit targeted genes with near specificity. See, Agrawal et al., 2003, “RNA interference: biology, mechanism, and applications,” Microbiol Mol Biol Rev. 67: 657-85; and Reynolds et al., 2004, “Rational siRNA design for RNA interference,” Nature Biotechnology 22, 326-330, each of which is hereby incorporated by reference. In some such embodiments, the perturbation is achieved by transfecting the siRNA into the one or more cells, DNA-vector mediated production, or viral-mediated siRNA synthesis. See, for example, Paddison et al., 2002, “Short hairpin RNAs (shRNAs) induce sequence-specific silencing in mammalian cells,” Genes Dev. 16:948-958; Sui et al., 2002, A DNA vector-based RNAi technology to suppress gene expression in mammalian cells,” Proc Natl Acad Sci USA 99:5515-5520; Brummelkamp et al., 2002, “A system for stable expression of short interfering RNAs in mammalian cells,” Science 296:550-553; Paddison et al., 2004, “Short hairpin activated gene silencing in mammalian cells,” Methods Mol Biol 265:85-100; Wong et al. 2003, “CIITAregulated plexin-A 1 affects T-cell-dendritic cell interactions, Nat Immunol 2003, 4:891-898; Tomar et al., 2003, “Use of adeno-associated viral vector for delivery of small interfering RNA. Oncogene 22:5712-5715; Rubinson et al., 2003 “A lentivirus-based system to functionally silence genes in primary mammalian cells, stem cells and transgenic mice by RNA interference,” Nat Genet 33:401-406; Moore et al., 2005, “Stable inhibition of hepatitis B virus proteins by small interfering RNA expressed from viral vectors,” J Gene Med; and Tran et al., 2003, “Expressing functional siRNAs in mammalian cells using convergent transcription, BMC Biotechnol 3:21; each of which is hereby incorporated by reference.

In some embodiments, a cell context is perturbed by exposure to a short hairpin RNA (shRNA). See, Taxman et al., 2006, “Criteria for effective design, construction, and gene knockdown by shRNA vectors,” BMC Biotechnology 6:7 (2006), which is hereby incorporated by reference. In some such embodiments, the perturbation is achieved by DNA-vector mediated production, or viral-mediated siRNA synthesis as generally discussed in the references cited above for siRNA.

In some embodiments, a cell context is perturbed by exposure to a single guide RNA (sgRNA) used in the context of palindromic repeat (e.g., CRISPR) technology. See, Sander and Young, 2014, “CRISPR-Cas systems for editing, regulating and targeting genomes,” Nature Biotechnology 32, 347-355, hereby incorporated by reference, in which a catalytically-dead Cas9 (usually denoted as dCas9) protein lacking endonuclease activity to regulate genes in an RNA-guided manner. Targeting specificity is determined by complementary base-pairing of a single guide RNA (sgRNA) to the genomic loci. sgRNA is a chimeric noncoding RNA that can be subdivided into three regions: a 20 nt base-pairing sequence, a 42 nt dCas9-binding hairpin and a 40 nt terminator. In some embodiments, when designing a synthetic sgRNA, only the 20 nt base-pairing sequence is modified from the overall template. In some such embodiments, the perturbation is achieved by DNA-vector mediated production, or viral-mediated sgRNA synthesis.

In some embodiments, a cell context is optimized for non-optical measurements of features, e.g., via RNASeq, L1000, proteomics, toxicity assays, publicly available bioassay data, in-house generated bioassays, microarrays, or chemical toxicity assays, etc.

In some embodiments, a cell context for a test state and corresponding query state is generated by perturbing a particular cell line with a cytokine or mixture of cytokines. See Heike and Nakahata, 2002, “Ex vivo expansion of hematopoietic stem cells by cytokines,” Biochim Biophys Acta 1592, 313-321, which is hereby incorporated by reference. In some embodiments the cell context includes cytokines (e.g., lymphokines, chemokines, interferons, tumor necrosis factors, etc.). In some embodiments a cell context includes lymphokines (e.g., Interleukin 2, Interleukin 3, Interleukin 4, Interleukin 5, Interleukin 6, granulocyte-macrophage colony-stimulating factor, interferon gamma, etc.). In some embodiments a cell context includes chemokines such as homeostatic chemokines (e.g., CCL 14, CCL19, CCL20, CCL21, CCL25, CCL27, CXCL12, CXCL13, etc.) and/or inflammatory chemokines (e.g., CXCL-8, CCL2, CCL3, CCL4, CCL5, CCL11, CXCL10). In some embodiments a cell context includes interferons (IFN) such as a type I IFN (e.g., IFN-α, IFN-β, IFN-ϵ, IFN-κ and IFN-ω), a type II IFN (e.g., IFN-γ), or a type III IFN. In some embodiments a cell context includes tumor necrosis factors such as TNFα or TNF alpha.

In some embodiments, a cell context for a test state and corresponding query state is generated by perturbing a particular cell line with a protein, such as a peptide aptamer. Peptide aptamers are combinatorial protein reagents that bind to target proteins with a high specificity and a strong affinity. By so doing, they can modulate the function of their cognate targets. In some embodiments, a peptide aptamer comprises one (or more) conformationally constrained short variable peptide domains, attached at both ends to a protein scaffold. In some embodiments, a cell context is perturbed with peptide aptamer derivatized with one or more functional moieties that can cause specific postranslational modification of their target proteins, or change the subcellular localization of the targets. See, for example, Colas et al., 2000, “Targeted modification and transportation of cellular proteins,” Proc. Natl. Acad. Sci. USA. 97 (25): 13720-13725, which is hereby incorporated by reference. In some embodiments, a cell context is perturbed with a peptide that selectively affects protein-protein interactions within an entity. In some such embodiments this protein-protein interaction affects an intracellular signaling event. See, for example, Souroujon and Mochly-Rosen, 1998, “Peptide modulators of protein-protein interactions in intracellular signaling,” Nature Biotechnology 16, 919-924, which is hereby incorporated by reference. In some embodiments, a cell context is perturbed with an antibody or other form of biologic.

In some embodiments, a cell context is generated by perturbing a particular cell line with a nucleic acid, such as a nucleic acid aptamer. Nucleic acid aptamers are short synthetic single-stranded oligonucleotides that specifically bind to various molecular targets such as small molecules, proteins, nucleic acids, and even cells and tissues. See, Ni el al., 2011, “Nucleic acid aptamers: clinical applications and promising new horizons,” Curr Med Chem 18(27), 4206, which is hereby incorporated by reference. In some instance nucleic acid aptamers are selected from a biopanning method such as SELEX (Systematic Evolution of Ligands by Exponential enrichment). See, Ellington and Szostak, 1990, “In vitro selection of RNA molecules that bind specific ligands,” Nature 346(6287), 818; and Tuerk and Gold, 1990, “Systematic evolution of ligands by exponential enrichment: RNA ligands to bacteriophage T4 DNA polymerase,” Science 249(4968), 505, each of which is hereby incorporated by reference. The SELEX screening method begins with a random sequence library of ssDNA or ssRNA that spans 20-100 nucleotides (nt) in length. The randomization of nucleic acid sequences provides a diversity of 4ⁿ, with n corresponding to the number of randomized bases. Diversities on the order of ˜10¹⁶ aptamers can typically generated and screened in the SELEX methods. Each random sequence region is flanked by constant sequences that is used for capture or priming. To overcome exonuclease degradation, aptamers can be chemically synthesized and capped with modified or inverted nucleotides to prevent terminal degradation. Modified oligonucleotides can also be incorporated within the aptamer, either during or after selection, for enhanced endonuclease stability. Some modified nucleotide triphosphates, particularly 2′-O-modified pyrimidines, can be efficiently incorporated into nucleic acid aptamer transcripts by T7 RNA polymerases. Common chemical modifications included during selection are 2′-amino pyrimidines and 2′-fluoro pyrimidines. See, Ni et al., 2011, “Nucleic acid aptamers: clinical applications and promising new horizons,” Curr Med Chem 18(27), 4206, which is hereby incorporated by reference.

In some embodiments, a cell context is generated by perturbing a particular cell line with a zinc finger transcription factor. In some such embodiments, the zinc finger protein transcription factor is encoded into vector that is transformed into the one or more cells, thereby causing the control of expression of one or more targeted components within the one or more cells. In some such embodiments, a sequence that is common to multiple (e.g., functionally related) components in the entity is used by a perturbation in the form of a zinc finger protein in order to control the transcription of all these component with a single perturbation in the form of a zinc finger transcription factor. In some embodiments, the perturbation in the form of a zinc finger transcription factor targets a family of related components in an entity by targeting and modulating the expression of the endogenous transcription factors that control them. See, for example, Doyon, 2008, “Heritable targeted gene disruption in zebrafish using designed zinc-finger nucleases,” Nature Biotechnology 26, 702-708, which is hereby incorporated by reference.

In some embodiments, a cell context is generated by introducing a mutation into the genome of a cell line, e.g., an insertion, deletion, inversion, transversion, etc. Generally, the mutation disrupts the expression or function of a target gene.

### Features

Each of the feature measurements 226, 230, and 234 used to form the basis of elements of vectors 246, 250, and 254, is selected from a plurality of measured features. In some embodiments, the one or more feature measurements include one or more of morphological features, expression data, genomic data, epigenomic data, epigenetic data, proteomic data, metabolomics data, toxicity data, bioassay data, etc.

In some embodiments, the corresponding set of elements in each vector includes between 5 test elements and 100,000 test elements. Likewise, in some embodiments, the corresponding set of elements includes a range of elements falling within the larger range discussed above, e.g., from 100 to 100,000, from 1000 to 100,000, from 10,000 to 100,000, from 5 to 10,000, from 100 to 10,000, from 1000 to 10,000, from 5 to 1000, from 100 to 1000, and the like. Generally, the more elements included in the data points, the more information available to distinguish the on-target and off-target effects of the query perturbations. On the other hand, as the number of elements in the set increases, the computational resources required to process the data and manipulate the multidimensional vectors also increases.

In some embodiments, each feature is an optical feature that is optically measured, e.g., using fluorescent labels (e.g., cell painting) or using native imaging, as described herein and known to the skilled artisan. In some embodiments, when each feature is an optical feature, a single image collection step (e.g., that obtains a single image or a series of images at multiple wavebands) can be used to collect image data from multiple samples, e.g., an entire multi-well plate. In some embodiments, a number of images are collected for each well in a multi-well plate. Feature extraction is then performed electronically from the collected image(s), limiting the experimental time required to extract features from a large plurality of cell contexts and compounds.

In some embodiments, a first subset of the features are optical features that are optically measured (e.g., e.g., using fluorescent labels (e.g., cell painting)), and a second subset of the features are non-optical features. Non-limiting examples of non-optical features include gene expression, protein levels, single endpoint bio-assays, metabolome data, microenvironment data, microbiome data, genome sequence and associated features (e.g., epigenetic data such as methylation, 3D genome structure, chromatin accessibility, etc.), and a relationship and/or change in a particular feature over time, e.g., within a single sample or across a plurality of samples in a time series. Further details about these and other types of non-optical features, as well as collection of data associated with these features, is provided below.

In some embodiments, each feature is a feature that is non-optically measured Non-limiting examples of non-optical features include gene expression, protein levels, single endpoint bio-assays, metabolome data, microenvironment data, microbiome data, genome sequence and associated features (e.g., epigenetic data such as methylation, 3D genome structure, chromatin accessibility, etc.), and a relationship and/or change in a particular feature over time, e.g., within a single sample or across a plurality of samples in a time series. Further details about these and other types of non-optical features, as well as collection of data associated with these features, is provided below. Thus, in some embodiments, multiple assays are performed for each instance (e.g., replicate) of a respective cell context that is exposed to a respective compound, e.g., both a nucleic acid microarray assay and a bioassay are performed from different instances of a respective cell context exposed to a respective compound.

In some embodiments, one or more of the features is determined from a non-cell-based assay. That is, in some embodiments, data collected from in vitro experiments performed in the absence of a cell is used in the construction of the multidimensional vectors described herein.

### Optically-Measured Features

In some embodiments, one or more of the features represent morphological features of a cell, or an enumerated portion of a cell, upon exposure of a respective compound in the cell context. Example features include, but are not limited to cell area, cell perimeter, cell aspect ratio, actin content, actin texture, cell solidity, cell extent, cell nuclear area, cell nuclear perimeter, cell nuclear aspect ratio, and algorithm-defined features (e.g., latent features). In some embodiment, example features include, but are not limited to, any of the features found in Table S2 of the reference Gustafsdottir S M, et al., PLoS ONE 8(12): e80999. doi:10.1371/journal.pone.0080999 (2013), which is hereby incorporated by reference.

In some embodiments, such morphological features are measured and acquired using the software program Cellprofiler. See Carpenter et al., 2006, “CellProfiler: image analysis software for identifying and quantifying cell phenotypes,” Genome Biol. 7, R100 PMID: 17076895; Kamentsky et al., 2011, “Improved structure, function, and compatibility for CellProfiler: modular high-throughput image analysis software,” Bioinformatics 2011/doi. PMID: 21349861 PMCID: PMC3072555; and Jones et al., 2008, CellProfiler Analyst: data exploration and analysis software for complex image-based screens, BMC Bioinformatics 9(1):482/doi: 10.1186/1471-2105-9-482. PMID: 19014601 PMCID: PMC261443, each of which is hereby incorporated by reference.

In some embodiments, the measurement of one or more feature is a fluorescent microscopy measurement of the different feature. In some embodiments, the one or more optical emitting compounds are dyes and where the vector for a compound in the plurality of compounds includes respective measurements of features in the plurality of features for the cell context in the presence of each of at least three different dyes. In some embodiments, the one or more optical emitting compounds are dyes and data points 276, 280, and 284 include respective measurements of features in the plurality of features for the cell context in the presence of each of at least five different dyes.

Accordingly, in some embodiments, one or more feature is measured after exposure of the cell context to the compound and to a panel of fluorescent stains that emit at different wavelengths, such as Concanavalin A/Alexa Fluor 488 conjugate (Invitrogen, cat. no. C11252), Hoechst 33342 (Invitrogen, cat. no. H3570), SYTO 14 green fluorescent nucleic acid stain (Invitrogen, cat. no. S7576), Phalloidin/Alexa Fluor 568 conjugate (Invitrogen, cat. no. A12380), and/or MitoTracker Deep Red (Invitrogen, cat. no. M22426). In some embodiments, measured features include one or more of staining intensities, textural patterns, size, and shape of the labeled cellular structures, as well as correlations between stains across channels, and adjacency relationships between cells and among intracellular structures. In some embodiments, two, three, four, five, six, seven, eight, nine, ten, or more than 10 fluorescent stains, imaged in two, three, four, five, six, seven, or eight channels, are used to measure features including different cellular components and/or compartments.

In some embodiments, one or more features are measured from single cells, groups of cells, and/or a field of view. In some embodiments, features are measured from a compartment or a component (e.g., nucleus, endoplasmic reticulum, nucleoli, cytoplasmic RNA, F-actin cytoskeleton, Golgi, plasma membrane, mitochondria) of a single cell. In some embodiments, each channel includes (i) an excitation wavelength range and (ii) a filter wavelength range in order to capture the emission of a particular dye from among the set of dyes the cell has been exposed to prior to measurement. An example of the dye that is being invoked and the type of cellular component that is measured as a features for five suitable channels is provided in Table 2 below, which is adapted from Table 1 of Bray et al., 2016, “Cell Painting, a high-content image-based assay for morphological profiling using multiplexed fluorescent dyes,” Nature Protocols, 11, p. 1757-74, which is hereby incorporated by reference.

TABLE 2
Example Channels Used for Measuring Features

| | | | | Entity |
|---|---|---|---|---|
| | | Filter | Filter | component or |
| Channel | Dye | (excitation; nm) | (emission; nm) | compartment |
| 1 | Hoechst 33342 | 387/11 | 417-477 | Nucleus |
| 2 | Concanavalin A/Alexa | 472/30a | 503-538a | Endoplasmic |
| | Fluor 488 conjugate | | | reticulum |
| 3 | SYTO 14 green | 531/40 | 573-613 | Nucleoli, |
| | fluorescent nucleic | | | cytoplasmic |
| | acid stain | | | RNAb |
| 4 | Phalloidin/Alexa Fluor | 562/40 | 622-662c | F-actin |
| | 568 conjugate, wheat- | | | cytoskeleton, |
| | germ agglutinin/Alexa | | | Golgi, plasma |
| | Fluor 555 conjugate | | | membrane |
| 5 | MitoTracker Deep Red | 628/40 | 672-712 | Mitochondria |

Cell Painting and related variants of cell painting represent another form of imaging technique that holds promise. Cell painting is a morphological profiling assay that multiplexes six fluorescent dyes, imaged in five channels, to reveal eight broadly relevant cellular components or organelles. Cells are plated in multi-well plates, perturbed with the treatments to be tested, stained, fixed, and imaged on a high-throughput microscope. Next, automated image analysis software identifies individual cells and measures any number between one and tens of thousands (but most often approximately 1,000) morphological features (various measures of size, shape, texture, intensity, etc. of various whole-cell and sub-cellular components) to produce a profile that is suitable for the detection of even subtle phenotypes. Profiles of cell populations treated with different experimental perturbations can be compared to suit many goals, such as identifying the phenotypic impact of chemical or genetic perturbations, grouping compounds and/or genes into functional pathways, and identifying signatures of disease. See, Bray et al., 2016, Nature Protocols 11, 1757-1774.

In some embodiments, the measurement of a feature is a label-free imaging measurement of the different feature. In some embodiments, one or more feature is measured by the label-free imaging technique after exposure of the cell context to a compound. Non-invasive, label free imaging techniques have emerged, fulfilling the requirements of minimal cell manipulation for cell based assays in a high content screening context. Among these label free techniques, digital holographic microscopy (Rappaz et al., 2015 Automated multi-parameter measurement of cardiomyocytes dynamics with digital holographic microscopy,” Opt. Express 23, 13333-13347) provides quantitative information that is automated for end-point and time-lapse imaging using 96- and 384-well plates. See, for example, Kuhn, J. 2013, et al., “Label-free cytotoxicity screening assay by digital holographic microscopy,” Assay Drug Dev. Technol. 11, 101-107; Rappaz et al., 2014 “Digital holographic microscopy: a quantitative label-free microscopy technique for phenotypic screening,” Comb. Chem. High Throughput Screen 17, 80-88; and Rappaz et al., 2015 in Label-Free Biosensor Methods in Drug Discovery (ed. Fang, Y.) 307-325, Springer Science+Business Media). Light sheet fluorescence microscopy (LSFM) holds promise for the analysis of large numbers of samples, in 3D high resolution and with fast recording speed and minimal photo-induced cell damage. LSFM has gained increasing popularity in various research areas, including neuroscience, plant and developmental biology, toxicology and drug discovery, although it is not yet adapted to an automated HTS setting. See, Pampaloni et al., 2014, “Tissue-culture light sheet fluorescence microscopy (TC-LSFM) allows long-term imaging of three-dimensional cell cultures under controlled conditions,” Integr. Biol. (Camb.) 6, 988-998; Swoger et al., 2014, “Imaging cellular spheroids with a single (selective) plane illumination microscope,” Cold Spring Harb. Protoc., 106-113; and Pampaloni et al., 2013, “High-resolution deep imaging of live cellular spheroids with light-sheet-based fluorescence microscopy,” Cell Tissue Res. 352, 161-177.

In some embodiments, the measurement of one or more features is a bright field measurement of the different feature. In some embodiments, one or more feature is measured by bright field microscopy after exposure of the cell context to a compound. In contrast to measurements obtained by fluorescent microscopy, which requires exposing the cell context to one of more fluorescent stain, bright field microscopy does not require the use of stains, reducing phototoxicity and simplifying imaging setup. Although the lack of stains reduces the contrast provided in bright field images, as compared to fluorescent images, various techniques have been developed to improve cellular imaging in this fashion. For example, Quantitative Phase Microscopy relies on estimation of a phase map generated from images acquired at different focal lengths. See, for example, Curl C L, et al., Cytometry A 65:88-92 (2005), which is incorporated by reference herein. Similarly, a phase map can be measured using lowpass digital filtering, followed by segmentation of individual cells. See, for example, Ali R., et al., Proc. 5th IEEE International Symposium on Biomedical Imaging: From Nano to Macro, ISBI:181-84 (2008), which is incorporated by reference herein. Texture analysis, e.g., where cell contours are extracted after segmentation, can also be used in conjunction with bright field microscopy. See, for example, Korzynska A, et al., Pattern Anal Appl 10:301-19 (2007). Yet other techniques are also available to facilitate use of bright filed microscopy, including z-projection based methods. See, for example, Selinummi J., et al., PLoS One, 4(10):e7497 (2009).

In some embodiments, the measurement of one or more features is phase contrast measurement of the different feature. In some embodiments, one or more feature is measured by phase contrast microscopy after exposure of the cell context to a compound. Images obtained by phase contrast or differential interference contrast (DIC) microscopy can be digitally reconstructed and quantified. See Koos, 2015, “DIC image reconstruction using an energy minimization framework to visualize optical path length distribution,” Sci. Rep. 6, 30420.

Although particular imaging techniques are specifically described herein, the methods provided herein could be performed using features measured from any of a number of microscope modalities.

In some embodiments, each feature represents a color, texture, or size of the cell context, or an enumerated portion of the cell context, upon exposure of the cell context to the amount of the respective compound. Example features include, but are not limited to cell area, cell perimeter, cell aspect ratio, actin content, actin texture, cell solidity, cell extent, cell nuclear area, cell nuclear perimeter, and cell nuclear aspect ratio. In some embodiment, example features include, but are not limited to, any of the features found in Table S2 of the reference Gustafsdottir S M, et al., PLoS ONE 8(12): e80999. doi:10.1371[journal.pone.0080999 (2013), which is hereby incorporated by reference.

In some embodiments, one or more of the measured features are latent features, e.g., extracted from an image of the cell context after exposure to the compound. In one embodiments, each respective instance of the plurality of instances of the cell context is imaged to form a corresponding two-dimensional pixelated image having a corresponding plurality of native pixel values and where a feature in the plurality of features comprises a result of a convolution or a series convolutions and pooling operators run against native pixel values in the plurality of native pixel values of the corresponding two-dimensional pixelated image. While this is an example of a latent feature that can be derived from an image, other latent features and mathematical combinations of latent features can also be used. A non-limiting example of the use of latent features in image-based profiling of cellular structure is found in Ljosa, V., et al., J Biomol. Screen., 18(10):10.1177/1087057113503553 (2013), which is incorporated herein by reference.

### Non-Optically-Measured Features

In some embodiments one or more of the measured features include expression data, e.g., obtained using a whole transcriptome shotgun sequencing (RNA-Seq) assay that quantifies gene expression from cells (e.g., a single cell) in counts of transcript reads mapped to gene constructs. As such, in some embodiments, RNA-Seq experiments aim at reconstructing all full-length mRNA transcripts concurrently from millions of short reads. RNA-Seq facilitates the ability to look at alternative gene spliced transcripts, post-transcriptional modifications, gene fusion, mutations/SNPs and changes in gene expression over time, or differences in gene expression in different groups or treatments. See, for example, Maher et al., 2009, “Transcriptome sequencing to detect gene fusions in cancer,” Nature. 458 (7234): 97-101, which is hereby incorporated by reference. In addition to mRNA transcripts, RNA-Seq can evaluate and quantify individual members of different populations of RNA including total RNA, mRNA, miRNA, IncRNA, snoRNA, or tRNA within entities. As such, in some embodiments, one or more of the features that is measured is an individual amount of a specific RNA species as determined using RNA-Seq techniques. In some embodiments, RNA-Seq experiments produce counts of component (e.g., digital counts of mRNA reads) that are affected by both biological and technical variation. In some embodiments RNA-Seq assembly is performed using the techniques disclosed in Li et al., 2008, “IsoLasso: A LASSO Regression Approach to RNA-Seq Based Transcriptome Assembly,” Cell 133, 523-536 which is hereby incorporated by reference.

In some embodiments one or more of the measured features are obtained using transcriptional profiling methods such an L1000 panel that measures a set of informative transcripts. In such an approach, ligation-mediated amplification (LMA) followed by capture of the amplification products on fluorescently addressed microspheres beads is extended to a multiplex reaction (e.g., a 1000-plex reaction). For instance, cells growing in 384-well plates are lysed and mRNA transcripts are captured on oligo-dT-coated plates. cDNAs are synthesized from captured transcripts and subjected to LMA using locus-specific oligonucleotides harboring a unique 24-mer barcode sequence and a 5′ biotin label. The biotinylated LMA products are detected by hybridization to polystyrene microspheres (beads) of distinct fluorescent color, each coupled to an oligonucleotide complementary to a barcode, and then stained with streptavidin-phycoerythrin. In this way, each bead can be analyzed both for its color (denoting landmark identity) and fluorescence intensity of the phycoerythrin signal (denoting landmark abundance). See Subramanian et al., “A Next Generation Connectivity Map: L1000 Platform and the First 1,000,000 Profiles,” Cell 171(6), 1437, which is hereby incorporated by reference. In some embodiments, between 500 and 1500 different informative transcripts are measured using this assay.

In some embodiments one or more of the measured features are obtained using microarrays. A microarray (also termed a DNA chip or biochip) is a collection of microscopic nucleic acid spots attached to a solid surface that can be used to measure the expression levels of large numbers of genes simultaneously. Each nucleic acid spot contains picomoles of a specific nucleic acid sequence, known as probes (or reporters or oligos). These can be a short section of a gene or other nucleic acid element that are used to hybridize a cDNA or cRNA (also called anti-sense RNA) sample (called target) under high-stringency conditions. For instance, by way of a non-limiting example, in some embodiments, the microarrays such as the Affymetrix GeneChip microarray, a high density oligonucleotide gene expression array, is used. Each gene on an Affymetrix microarray GeneChip is typically represented by a probe set consisting of 11 different pairs of 25-bp oligos covering features of the transcribed region of that gene. Each pair consists of a perfect match (PM) and a mismatch (MM) oligonucleotide. The PM probe exactly matches the sequence of a particular standard genotype, often one parent of a cross, while the MM differs in a single substitution in the central, 13” base. The MM probe is designed to distinguish noise caused by non-specific hybridization from the specific hybridization signal. See, Jiang, 2008, “Methods for evaluating gene expression from Affymetrix microarray datasets,” BMC Bioinformatics 9, 284, which is hereby incorporated by reference.

In some embodiments one or more of the measured features are obtained using ChIP-Seq data. See, for example, Quigley and Kintner, 2017, “Rfx2 Stabilizes Foxj1 Binding at Chromatin Loops to Enable Multiciliated Cell Gene Expression,” PLoS Genet 13, e1006538, which is hereby incorporated by reference. In some embodiments, ChIP-seq is used to determine how transcription factors and other chromatin-associated proteins influence phenotype-affecting mechanisms in entities (e.g., cells). Specific DNA sites in direct physical interaction with transcription factors and other proteins can be isolated by chromatin immunoprecipitation. ChIP produces a library of target DNA sites bound to a protein of interest (component) in vivo. Parallel sequence analyses are then used in conjunction with whole-genome sequence databases to analyze the interaction pattern of any protein with DNA (Johnson et al., 2007, “Genome-wide mapping of in vivo protein-DNA interactions,” Science. 316: 1497-1502, which is hereby incorporated by reference) or the pattern of any epigenetic chromatin modifications. This can be applied to the set of ChIP-able proteins and modifications, such as transcription factors, polymerases and transcriptional machinery, structural proteins, protein modifications, and DNA modifications.

ChIP selectively enriches for DNA sequences bound by a particular protein (component) in living cells (entities). The ChIP process enriches specific cross-linked DNA-protein complexes using an antibody against the protein (component) of interest. Oligonucleotide adaptors are then added to the small stretches of DNA that were bound to the protein of interest to enable massively parallel sequencing. After size selection, all the resulting ChIP-DNA fragments are sequenced concurrently using a genome sequencer. A single sequencing run can scan for genome-wide associations with high resolution, meaning that features can be located precisely on the chromosomes. Various sequencing methods can be used. In some embodiments the sequences are analyzed using cluster amplification of adapter-ligated ChIP DNA fragments on a solid flow cell substrate to create clusters of clonal copies. The resulting high density array of template clusters on the flow cell surface is sequenced by a Genome analyzing program. Each template cluster undergoes sequencing-by-synthesis in parallel using fluorescently labelled reversible terminator nucleotides. Templates are sequenced base-by-base during each read. Then, the data collection and analysis software aligns sample sequences to a known genomic sequence to identify the ChIP-DNA fragments.

In some embodiments one or more of the measured features are obtained using ATAC-seq (Assay for Transposase-Accessible Chromatin using sequencing), which is a technique used in molecular biology to study chromatin accessibility. See Buenrostro et al., 2013, “Transposition of native chromatin for fast and sensitive epigenomic profiling of open chromatin, DNA-binding proteins and nucleosome position,” Nature Methods 10, 1213-1218, which is hereby incorporated by reference. In some embodiments, ATAC-seq make use of the action of the transposase Tn5 on the genomic DNA of an entity. See, for example, Buenrostro et al., 2015, “ATAC-seq: A Method for Assaying Chromatin Accessibility Genome-Wide,” Current Protocols in Molecular Biology: 21.29.1-21.29.9, which is hereby incorporated by reference. Transposases are enzymes catalyzing the movement of transposons to other parts in the genome. While naturally occurring transposases have a low level of activity, ATAC-seq employs a mutated hyperactive transposase. The high activity allows for highly efficient cutting of exposed DNA and simultaneous ligation of specific sequences, called adapters. Adapter-ligated DNA fragments are then isolated, amplified by PCR and used for next generation sequencing. See Buenrostro et al., 2013, “Transposition of native chromatin for fast and sensitive epigenomic profiling of open chromatin, DNA-binding proteins and nucleosome position,” Nature Methods 10, 1213-1218, which is hereby incorporated by reference.

While not intending to be limited to any particular theory, transposons are believed to incorporate preferentially into genomic regions free of nucleosomes (nucleosome-free regions) or stretches of exposed DNA in general. Thus enrichment of sequences from certain loci in the genome indicates absence of DNA-binding proteins or nucleosome in the region. An ATAC-seq experiment will typically produce millions of next generation sequencing reads that can be successfully mapped on the reference genome. After elimination of duplicates, each sequencing read points to a position on the genome where one transposition (or cutting) event took place during the experiment. One can then assign a cut count for each genomic position and create a signal with base-pair resolution. This signal is used as a features in some embodiments of the present disclosure. Regions of the genome where DNA was accessible during the experiment will contain significantly more sequencing reads (since that is where the transposase preferentially acts), and form peaks in the ATAC-seq signal that are detectable with peak calling tools. In some embodiments, such peaks, and their locations in the genome are used as features. In some embodiments, these regions are further categorized into the various regulatory element types (e.g., promoters, enhancers, insulators, etc.) by integrating further genomic and epigenomic data such as information about histone modifications or evidence for active transcription. Inside the regions where the ATAC-seq signal is enriched, one can also observe sub-regions with depleted signal. These sub-regions, typically only a few base pairs long, are considered to be “footprints” of DNA-binding proteins. In some embodiments, such footprints, or their absence or presence thereof are used as features.

In some embodiments flow cytometry methods using Luminex beads, are used to obtain values for one or more of the measured features. See for example, Susal et al., 2013, Transfus Med Hemother 40, 190-195, which is hereby incorporated by reference. For instance, the Luminex-supported single antigen bead (L-SAB) test allows for the characterization of human leukocyte antigen (HLA) antibody specificities. In such a flow cytometric method, microbeads coated with recombinant single antigen HLA molecules are employed in order to differentiate antibody reactivity in two reaction tubes against 100 different HLA class I and 100 different HLA class II alleles. An approximation of the strength of antibody reactivity is derived from the mean fluorescence intensity (MFI) and in some embodiments this serves as features in the present disclosure. In addition to antibody reactivity against HLA-A, -B, -C, -DR and -DQB antigens, L-SAB is capable of detecting antibodies against HLA-DQA, -DPA, and -DPB antigens. In some embodiments, other Luminex kits are used for detection of non-HLA antibodies in order to derive values for one or more features for entities in accordance with the present disclosure. For instance, in some embodiments, major histocompatibility complex class I-related chain A (MICA) and human neutrophil antibodies, and kits that utilize, instead of recombinant HLA molecules, affinity purified pooled human HLA molecules obtained from multiple cell lines (screening test to detect presence of HLA antibodies without further specification) or phenotype panels in which each bead population bears either HLA class I or HLA class II proteins of a cell lines derived from a single individual (panel reactivity, PRA-test) are used to determine value for features for entities in accordance with an embodiment of the present disclosure.

In some embodiments, flow cytometry methods, such fluorescent cell barcoding, is used to obtain values for one or more of the measured features. Fluorescent cell barcoding (FCB) enables high throughput, e.g., high content flow cytometry by multiplexing samples of entities prior to staining and acquisition on the cytometer. Individual cell samples (entities) are barcoded, or labeled, with unique signatures of fluorescent dyes so that they can be mixed together, stained, and analyzed as a single sample. By mixing samples prior to staining, antibody consumption is typically reduced 10 to 100-fold. In addition, data robustness is increased through the combination of control and treated samples, which minimizes pipetting error, staining variation, and the need for normalization. Finally, speed of acquisition is enhanced, enabling large profiling experiments to be run with standard cytometer hardware. See, for example, Krutzik, 2011, “Fluorescent Cell Barcoding for Multiplex Flow Cytometry,” Curr Protoc Cytom Chapter 6: Unit 6.31, which is hereby incorporated by reference.

In some embodiments, metabolomics is used to obtain values for one or more of the features. Metabolomics is a systematic evaluation of small molecules in order to obtain biochemical insight into disease pathways. In some embodiments, such metabolomics comprises evaluation of plasma metabolomics in diabetes (Newgard et al., 2009, “A branched-chain amino acid-related metabolic signature that differentiates obese and lean humans and contributes to insulin resistance,” Cell Metab 9: 311-326, 2009) and ESRD (Wang, 2011, “RE: Metabolite profiles and the risk of developing diabetes,” Nat Med 17: 448-453). In some embodiments, urine metabolomics is used to obtain values for one or more of the features. Urine metabolomics offers a wider range of measurable metabolites because the kidney is responsible for concentrating a variety of metabolites and excreting them in the urine. In addition, urine metabolomics may offer direct insights into biochemical pathways linked to kidney dysfunction. See, for example, Sharma, 2013, “Metabolomics Reveals Signature of Mitochondrial Dysfunction in Diabetic Kidney Disease,” J Am Soc Nephrol 24, 1901-12, which is hereby incorporated by reference.

In some embodiments, mass spectrometry is used to obtain values for one or more of the measured features. For instance, in some embodiments, protein mass spectrometry is used to obtain values for one or more of the measured features. In particular, in some embodiments, biochemical fractionation of native macromolecular assemblies within entities followed by tandem mass spectrometry is used to obtain values for one or more of the measured features. See, for example, Wan et al., 2015, “Panorama of ancient metazoan macromolecular complexes,” Nature 525, 339-344, which is hereby incorporated by reference. Tandem mass spectrometry, also known as MS/MS or MS2, involves multiple steps of mass spectrometry selection, with some form of fragmentation occurring in between the stages. In a tandem mass spectrometer, ions are formed in the ion source and separated by mass-to-charge ratio in the first stage of mass spectrometry (MS1). Ions of a particular mass-to-charge ratio (precursor ions) are selected and fragment ions (product ions) are created by collision-induced dissociation, ion-molecule reaction, photodissociation, or other process. The resulting ions are then separated and detected in a second stage of mass spectrometry (MS2). In some embodiments the detection and/or presence of such ions serve as the one or more of the measured features.

In some embodiments, the features that are observed for an entity or a plurality of entities are post-translational modifications that modulate activity of proteins within a cell. In some such embodiments, mass spectrometric peptide sequencing and analysis technologies are used to detect and identify such post-translational modifications. In some embodiments, isotope labeling strategies in combination with mass spectrometry are used to study the dynamics of modifications and this serves as a measured feature. See for example, Mann and Jensen, 2003 “Proteomic analysis of post-translational modifications,” Nature Biotechnology 21, 255-261, which is hereby incorporated by reference. In some embodiments, mass spectrometry is user to determine splice variants in entities, for instance, splice variants of components within entities, and such splice variants and the detection of such splice variants serve as measured features. See for example, Nilsen and Graveley, 2010, “Expansion of the eukaryotic proteome by alternative splicing, 2010, Nature 463, 457-463, which is hereby incorporated by reference.

In some embodiments, imaging cytometry is used to obtain values for one or more of the measured features. Imaging flow cytometry combines the statistical power and fluorescence sensitivity of standard flow cytometry with the spatial resolution and quantitative morphology of digital microscopy. See, for example, Basiji et al., 2007, “Cellular Image Analysis and Imaging by Flow Cytometry,” Clinics in Laboratory Medicine 27, 653-670, which is hereby incorporated by reference.

In some embodiments, electrophysiology is used to obtain values for one or more of the measured features. See, for example, Dunlop et al., 2008, “High-throughput electrophysiology: an emerging paradigm for ion-channel screening and physiology,” Nature Reviews Drug Discovery 7, 358-368, which is hereby incorporated by reference.

In some embodiments, proteomic imaging/3D imaging is used to obtain values for one or more of the measured features. See for example, United States Patent Publication No. 20170276686 A1, entitled “Single Molecule Peptide Sequencing,” which is hereby incorporated by reference. Such methods can be used to large-scale sequencing of single peptides in a mixture from an entity, or a plurality of entities at the single molecule level.

### Assay Parameters

As described herein with reference to FIG. 3, in some embodiments, each feature measurement is obtained in replicate, e.g., each condition (e.g., each control state, teste state, and/or query state) is performed more than once and each feature measurement is obtained from each instance of the condition. In some embodiments, feature measurements are obtained from at least 2, 3, 4, 5, 6, 7, 8, 9, 10, 15, 20, 25, 50, 75, 100, 500, or more instances of every condition, e.g., experimental conditions are prepared in two or more replicates.

Similarly, as described herein with reference to FIG. 3, in some embodiments, each query perturbation (e.g., compound) is exposed to each cell context at a plurality of concentrations. In some embodiments, each query perturbation (e.g., compound) is exposed to each cell context using at least 2, 3, 4, 5, 6, 7, 8, 9, 10, or more concentrations. Similarly, in some embodiments, each feature measurement is obtained at each concentration in replicate.

With respect to the concentrations of compounds used for any particular query perturbation, the skilled artisan will know how to select a concentration for a given compound. In some embodiments, each compound will be used at the same concentrations. In some embodiments, different compounds will be used at different concentrations, e.g., based upon one or more known or expected property of the compound such as molecular weight, solubility, presence or particular functional groups, known or expected interactions, known or expected toxicity, etc. For example, in some embodiments, where a respective compound is known to be toxic to a cell type used in a particular cell context, the concentration of the compound may be adjusted, e.g., relative to the concentration used for other compounds. Generally, the time over which a cell context is exposed to a compound is influenced by the particular feature being measured and/or the particular assay from which the feature data is being generated. For example, where the assay being used measures a phenomenon that occurs rapidly following exposure of the cell context to the compound, the cell context does not need to be exposed to the compound for a long period of time prior to measurement of the feature. Conversely, where the assay being used measures a phenomenon that occurs slowly, or after a significant delay, following exposure of the cell context to the compound, a longer incubation time should be used prior to measuring the feature.

In some embodiments, e.g., where latent features are being extracted from a cell context, the time over which the cell context is exposed to a compound prior to measurement is determined stochastically. In some embodiments, the time over which the cell context is exposed to a compound prior to measurement is determined based on experience or trial and error with a particular assay or phenomenon. In one embodiment, exposure of the amount of the respective compound to the cell context is for at least one hour prior to obtaining the measurement. In some embodiments, the measurement is obtained by cellular imaging, e.g., using fluorescent labels (e.g., cell painting) or using native imaging, as described herein and known to the skilled artisan. In some embodiments, exposure of the amount of the respective compound to the cell context is for at least one hour prior to obtaining an image.

In some embodiments feature data is acquired using an automated cellular imaging system (e.g., ImageXpress Micro, Molecular Devices), where cell contexts have been arranged in multi-well plates (e.g., 384-well plates) after they have been stained with a panel of dyes that emit at different discrete wavelengths (e.g., Hoechst 33342, Alexa Fluor 594 phalloidin, etc.) and exposed to a perturbation. In some embodiments the cell contexts are imaged with an exposure that is a determined by the marker dye used (e.g., 15 ms for Hoechst, 1000 ms for phalloidin), at 20× magnification with 2× binning. For each well, in some embodiments the optimal focus is found using laser auto-focusing on a particular dye channel (e.g., the Hoechst channel). In some embodiments the automated microscope is then programmed to collect a z-stack of 32 images (z=0 at the optimal focal plane, 16 images above the focal plane, 16 below) with 2 sm between slices. In some embodiments each well contains several thousand cells in them, and thus each digital representation of a well captured by a camera represents several thousand cells in each of several different wells. In some embodiments, segmentation software is used to identify individual cells in the digital images and moreover various components (e.g., cellular components) within individual cells. Once the cellular components are segmented and identified, mathematical transformations are performed on these components on order to obtain the measurements of features.

### Dimensional Reduction

In some embodiments, the variability model is a dimensional reduction technique that uses a statistical feature selection or feature extraction procedure known in the art, for example, principal component analysis, non-negative matrix factorization, kernel PCA, graph-based kernel PCA, linear discriminant analysis, generalized discriminant analysis, and use of an autoencoder. This, in turn, reduces the computational burden of analyzing the data set by compressing the data in order to make the method more computationally efficient, e.g., by allowing the computer to apply an algorithm to the smaller dataset rather than the full dataset.

Principle component analysis (PCA) reduces the dimensionality of a multidimensional data point by transforming the plurality of elements (e.g., measured elements 226, 230, and/or 234) to a new set of variables (principal components) that summarize the features of the training set. See, for example, Jolliffe, 1986, Principal Component Analysis, Springer, New York, which is hereby incorporated by reference. PCA is also described in Draghici, 2003, Data Analysis Tools for DNA Microarrays, Chapman & Hall/CRC, which is hereby incorporated by reference. Principal components (PCs) are uncorrelated and are ordered such that the kth PC has the kth largest variance among PCs across the observed data for the features. The kth PC can be interpreted as the direction that maximizes the variation of the projections of the data points such that it is orthogonal to the first k−1 PCs. The first few PCs capture most of the variation in the observed data. In contrast, the last few PCs are often assumed to capture only the residual “noise” in the observed data. As such, the principal components derived from PCA can serve as the basis of vectors that are used in accordance with the present disclosure.

Non-negative matrix factorization and non-negative matrix approximation reduce the dimensionality of a multidimensional matrix by factoring the matrix into two matrices, each of which have significantly lower dimensionality, but which provide a product having the same, or approximately the same, dimensionality as the original higher-dimensional matrix. See, for example, Lee and Seung, “Learning the parts of objects by non-negative matrix factorization, Nature, 401(6755):788-91 (1999), which is hereby incorporated by reference. See also Dhillon and Sra, “Generalized Nonnegative Matrix Approximations with Bregman Divergences,” Advances in Neural Information Processing Systems 18 (NIPS 2005), which is hereby incorporated by reference.

Kernel PCA is an extension of PCA in which N elements of a vector are mapped onto a N-dimensional space using a non-trivial, arbitrary function, creating projections of the elements onto principle components lying on a lower dimensional subspace. In this fashion, kernel PCA is better equipped than PCA to reduce the dimensionality of non-linear data. See, for example, Scholkopf, “Nonlinear Component Analysis as a Kernel Eigenvalue Problem,” Neural Computation, 10: 1299-1319 (198), which is hereby incorporated by reference.

Linear discriminant analysis (LDA), like PCA, reduces the dimensionality of a multidimensional vector by transforming the plurality of elements (e.g., measured elements 216) to a new set of variables (principal components) that summarize the features of the training set. However, unlike PCA, LDA is a supervised feature extraction method which (i) calculates between-class variance, (ii) calculates within-class variance, and then (iii) constructs a lower dimensional-representation that maximizes between-class variance and minimizes within-class variance. See, for example, Tharwat, A., et al., “Linear discriminant analysis: A detailed tutorial,” AI Communications, 30:169-90 (2017), which is hereby incorporated by reference.

Generalized discriminant analysis (GDA), similar to kernel PCA, maps non-linear input elements of multidimensional vectors into higher-dimensional space to provide linear properties of the elements, which can then be analyzed according to classical linear discriminant analysis. In this fashion, GDA is better equipped than LDA to reduce the dimensionality of non-linear data. See, for example, Baudat and Anouar, “Generalized Discriminant Analysis Using a Kernel Approach,” Neural Comput., 12(10):2385-404 (2000).

Autoencoders are artificial neural networks used to learn efficient data codings in an unsupervised learning algorithm that applies backpropagation. Autoencoders consist of two parts, an encoder and a decoder. The encoder reads an input vector and compress it to a lower-dimensional vector, and the decoder reads the compressed vector and recreates the input vector. See, for example, Chapter 14 of Goodfellow et al., “Deep Learning,” MIT Press (2016), which is hereby incorporated by reference.

Yet other dimension reductions techniques known in the art may also be applied to the methods described herein. For example, in some embodiments, a subset of measured features is selected for inclusion in a reduced dimension representation of a data point, while discarding other features, e.g., based on optimality criterion in linear regression. See, for example, Draper and Smith, “Applied Regression Analysis,” 2d Edition, New York: John Wiley & Sons, Inc. (1981), which is hereby incorporated by reference. Similarly, in some embodiments, discrete methods, in which features are either selected or discarded, e.g., a leaps and bounds procedure, are used. See, for example, Furnival and Wilson, “Regressions by Leaps and Bounds,” Technometrics, 16(4):499-511 (1974), which is hereby incorporated by reference. Likewise, in some embodiments, linear regression by forward selection, backward elimination, or bidirectionsl elimination are used. See, for example, Draper and Smith, “Applied Regression Analysis,” 2d Edition, New York: John Wiley & Sons, Inc. (1981). In yet other embodiments, shrinkage methods, e.g., methods that reduce/shrink the redundant or irrelevant features in a more continuous fashion are used, e.g., ridge regression, Lasso, and Derived Input Direction Methods (e.g., PCR, PLS).

### First Example Embodiment

According to a first example embodiment, an image set for cellular morphological variation across many experimental batches. High-throughput screening techniques are in common use in many fields of biology, however it is well-known that measurements from high-throughput screens are confounded by the introduction of non-biological artifacts that arise from variability in the technical execution of different experimental batches. These batch effects are known to obscure biological conclusions and it is therefore necessary to correct for them. While a number of techniques have been proposed, to our knowledge there is not a publicly-available biological dataset that was designed specifically to systematically study batch effect correction. To this end, a set of 125,568 high-resolution fluorescence microscopy images of human cells under more than 1,100 genetic perturbations in 51 experimental batches across four cell types. A visual inspection of the images by batch makes it clear that the set indeed demonstrates significant batch effects. The image set in detail. A classification task is designed to study batch effect correction on these images, and provide some baseline results for the task. The images will further development of effective methods for removing batch effects that generalize well to unseen experimental batches and share these methods with the scientific community.

High-throughput screening techniques are in common use in many biological fields, including genetics (Echeverri & Perrimon, 2006; Zhou et al., 2014) and drug discovery (Broach et al., 1996; Macarron et al., 2011; Swinney & Anthony, 2011; Boutros et al., 2015). Such techniques are capable of generating large amounts of data that, when coupled with modern machine learning methods, could help in answering fundamental questions in biology, and addressing serious issues such as the exponential rise in the cost of developing an approved drug, which is now estimated to be well over $2 billion (Scannell et al., 2012; DiMasi et al., 2016). However, creating such large volumes of biological data necessarily requires the data to be generated in experimental batches, or groups of experiments that are executed at similar times under similar conditions. Even when experiments are carefully designed to control for technical variables such as temperature, humidity, and reagent concentration, the measurements taken from these screens are confounded by non-biological artifacts that arise from variability in the technical execution of each batch. These batch effects create factors of variation within the data that are irrelevant to the biological variables under study, but are unfortunately often correlated with them. It is therefore necessary to correct for batch effects before drawing any biological conclusions from measurements taken from high-throughput screens (Leek et al., 2010; Parker & Leek, 2012; Soneson et al., 2014; Nygaard et al., 2016).

Many computational methods have been designed for dealing with batch effects (Leek et al., 2010; Chen et al., 2011; Lazar et al., 2012; Parker & Leek, 2012; Leek et al., 2012; Goh et al., 2017; Shaham et al., 2017), yet there do not seem to be any publicly-available biological datasets that were systematically created to study them. Here such a dataset is provided, consisting of images of human cells under more than 1,100 different genetic perturbations across 51 experimental batches and four cell types. A machine learning task is it utilized which that gauges the effectiveness of the batch effect correction method-correctly classify the genetic perturbation present in each image in a held-out set of batches. Thus, in order for the classifier to generalize to unseen batches, it must learn to separate biological and technical factors in test images and make predictions only on the biological factors.

This dataset and task will be of interest to the rapidly growing community of researchers applying machine learning methods to complex biological data sets, especially those working with high-throughput phenotypic screens (Angermueller et al., 2016; Kraus et al., 2016; Caicedo et al., 2017; Kraus et al., 2017; Ando et al., 2017; Chen et al., 2018). The specific task of removing batch effects is relevant to the broader life sciences community and can provide insights that enable researchers to develop improved methods for working with other biological datasets. In addition, the dataset is of interest to the larger community of machine learning researchers working in computer vision, especially those in the areas of domain adaptation, transfer learning, and k-shot learning.

### Description of an Example Dataset

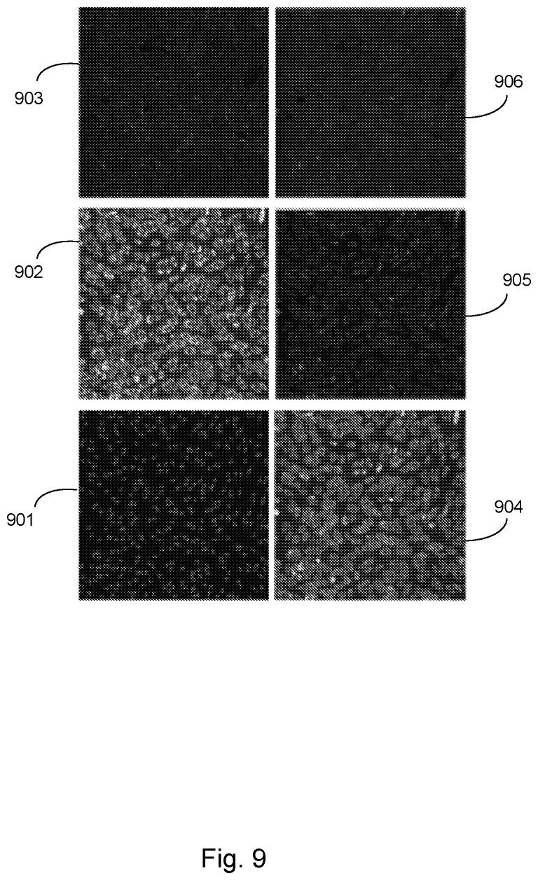

The image set was produced by automated high-throughput screening. It is comprised of fluorescence microscopy images of human cells of four different types-HUVEC, RPE, HepG2, and U2OS—which were acquired using a 6-channel variation of the Cell Painting imaging protocol (Bray et al., 2016). An example image is provided in FIG. 9.

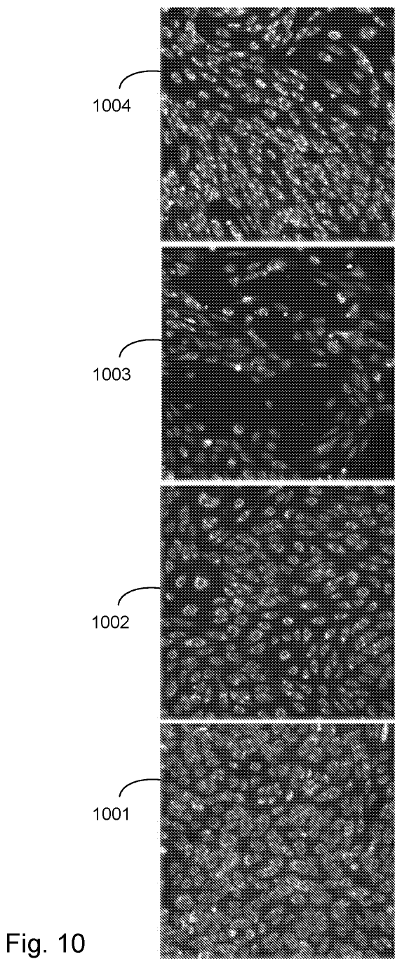

FIG. 9 shows 6-channel faux-colored composite image of HUVEC cells and individual channels: nuclei (blue) 901, endoplasmic reticuli (green) 902, actin (red) 903, nucleoli and cytoplasmic RNA (cyan) 904, mitochondria (magenta) 905, and Golgi (yellow) 906. The similarity in content between some channels is due in part to the spectral overlap between the fluorescent stains used in those channels. The six channels of an image illuminate the different parts of the cell population in the field of view: nuclei, endoplasmic reticuli, actin, nucleoli and cytoplasmic RNA, mitochondria, and Golgi. The images themselves are the result of running 51 different instances of the same type of experiment. Each experiment instance is comprised of four 384-well plates (see FIG. 3), used to isolate populations of cells into wells. The wells are laid out on each plate in a 16×24 grid, but only the wells in the inner 14×22 grid are used. Of these 308 usable wells, one remains untreated to provide a negative control. The rest of the 307 wells receive exactly one small interfering ribonucleic acid, or siRNA, at a fixed concentration (Tuschl, 2001). Each siRNA is designed to knockdown a single target gene via the RNA interference pathway, reducing the expression of the gene and its associated protein. However, siRNAs are known to have significant but consistent off-target effects via the microRNA pathway, creating partial knockdown of many other genes as well. The overall effect of siRNA transfection is to perturb the morphology, count, and distribution of cells in each well, creating a distinct phenotype associated with each siRNA. The phenotype is sometimes visually recognizable from the images, but often the difference in cell morphology is subtle and hard to detect visually (see FIG. 10).

FIG. 10 shows images of four different siRNA phenotypes (1001, 1002, 1003, and 1004). These images are from the same plate in a HUVEC experiment, such as the one described in conjunction with FIG. 9.

In each experiment, the same 30 siRNA appear on every plate as a positive control for the plate. These control siRNA target different genes and produce a variety of phenotypic effects so that, when combined with the single untreated well already mentioned, they provide a set of useful reference wells per plate. The 1,108 remaining wells of each experiment (277 wells×4 plates) receive 1,108 different siRNA, respectively. These non-control siRNA target different genes than each other and the genes of the control siRNA. Notice that while the control siRNA appears on each plate, each non-control siRNA appears at most once in each experiment. Although rare, it happens that either an siRNA is not transferred into its well, resulting in an additional untreated well, or an operational error renders the well unsuitable for inclusion in the dataset.

When the images were originally acquired from the microscope, they were of spatial resolution 2048×2048, but in order to make the dataset more manageable, they were downsampled by a sidelength factor of 2 and cropped to the center 512×512 field of view. The image set contains two non-overlapping 512×512 fields of view per well. Therefore, there could be as many as 125,664 images (=51 experiments×4 plates/experiment×4 wells/plate×2 images/well), but, because of operational errors, 48 wells were removed in total, resulting in 125,568 actual images in the dataset.

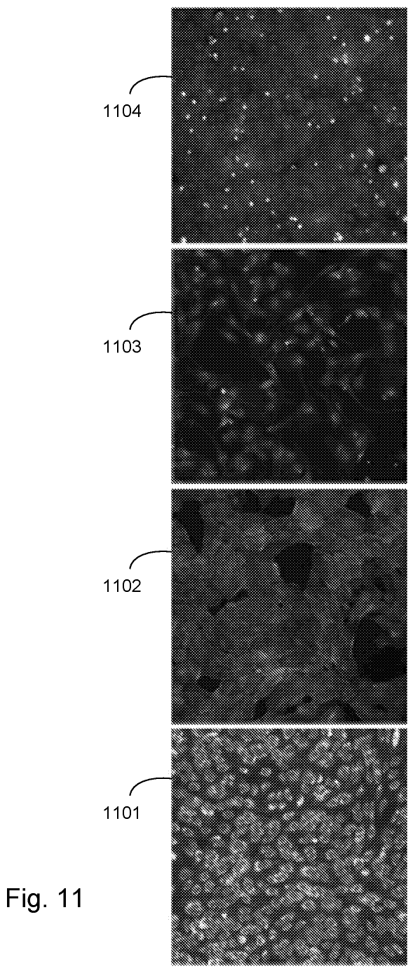

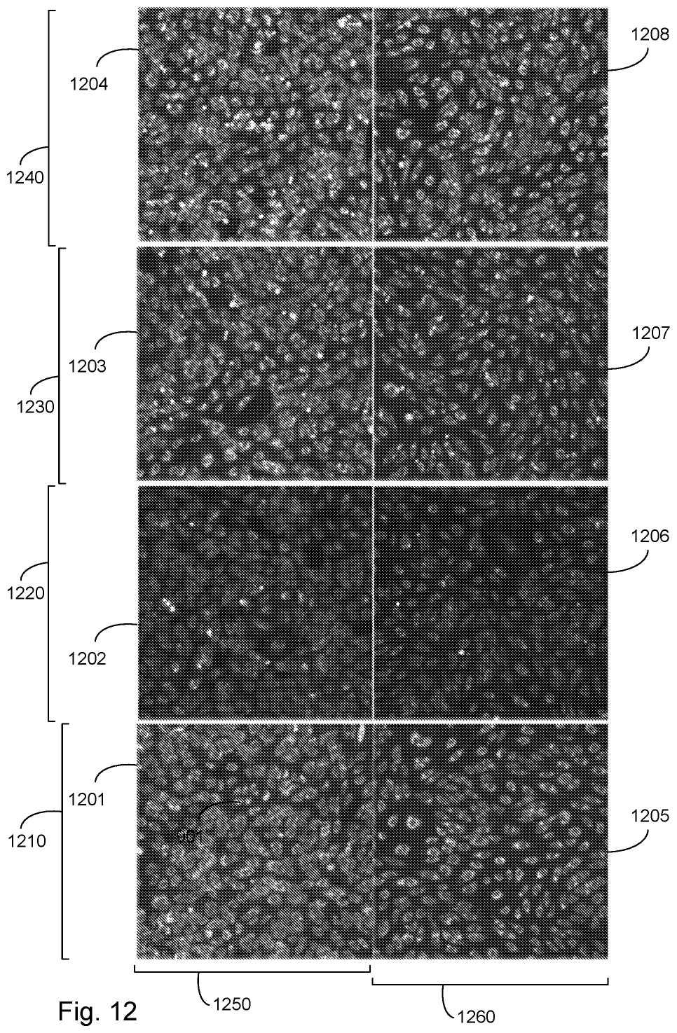

As was mentioned, the entire dataset consists of 51 experiments: 24 in HUVEC, 11 in RPE, 11 in HepG2, and 5 in U2OS. FIG. 11 shows the phenotype of a single siRNA in the four different cell types (1101, 1102, 1103, 1104). Each of the 51 experiments was run in a different batch, and the batches were executed at least one week apart from each other, resulting in images that exhibit technical effects common to their batch and distinct from other batches (see FIG. 12). It is this feature of the dataset that makes it particularly suited for studying batch effects and methods for correcting them.

FIG. 12 shows images of two different siRNA (rows 1250 and 1260) in HUVEC cells across four experimental batches (columns 1210, 1220, 1230, and 1240). Notice the visual similarity of images from the same batch. For example, images 1201 and 1205 are similar; images 1202 and 1206 are similar, images 1203 and 1207 are similar; and images 1204 and 1208 are similar.

In some embodiments, the image set is accompanied by metadata providing the following information about each image: cell type, experiment, plate, well location, and treatment class (1,138 siRNA classes plus one untreated class).

### Example Classification Task and Baseline Results

While the dataset is useful for many studies, the following task for studying batch effect correction is suggested: correctly classify the images of non-control siRNA in a hold-out set of batches. In order for a classifier to generalize well to unseen batches, it must learn to separate biological factors associated with siRNA perturbation from technical factors associated with batch effects in the training batches, and use only the biological factors for classification. In order to provide a baseline for this task, 33 experiments were randomly chosen for training (16 HUVEC, 7 RPE, 7 HepG2, 3 U2OS) and 9 for testing (4 HUVEC, 2 RPE, 2 HepG2, 1 U2OS), and trained a standard ResNet50 (He et al., 2016) on just the 1,108 non-control siRNA in the training set. The images were not preprocessed in any way, nor were any of the control images used at all. While training accuracies all reached near 100%, the average test accuracy was 24.4% over two splits of the data. To assess the extent to which batch effects are affecting these results, we split the data again into training and test sets of sizes similar to the first split, but without taking batch into account. The average test accuracy in this case was 32.8%, which is 34% higher than when we split by batch, demonstrating that batch effects have a significant impact on the models ability to classify. A summary of these results, including results on individual cell types, is presented in Table 3.

TABLE 3
Average Test Accuracies Per Cell Type and Data Split.

| Split | HUVEC | RPE | HepG2 | U2OS | All |
|---|---|---|---|---|---|
| Batch | 45.8% ± 15.5 | 20.0% ± 19.9 | 14.1% ± 4.9 | 0.0% ± 0.0 | 24.4% ± 1.5 |
| Random | 57.9% ± 0.0 | 33.2% ± 0.0 | 16.7% ± 0.0 | 4.2% ± 0.0 | 32.8% ± 0.0 |

### Succinct Descriptions of Various Aspects

In one aspect of a computer system for evaluating an effect of one or more perturbations on cells of a first cell type, the computer system comprising: one or more processors; a memory; and one or more programs, wherein the one or more programs are stored in the memory and are configured to be executed by the one or more processors, the one or more programs include instructions for: obtaining a screen definition for a screen, wherein the screen comprises a cell-based assay that is run on a temporarily contiguous basis using a plurality of multi-well plates, the screen definition identifies a first plurality of control wells and a plurality of data wells in the plurality of multi-well plates, each respective control well in the first plurality of control wells is labeled with a control perturbation label corresponding to a control perturbation in a first plurality of control perturbations that is independently included in the respective control well, each respective data well in the plurality of data wells is labeled with a data perturbation label corresponding to a data perturbation in a plurality of data perturbations that is independently included in the respective data well, and an aliquot of cells of the first cell type is included in each control well in the first plurality of control wells and in each data well in the plurality of data wells. The one or more programs further include instructions for obtaining, for each respective control well in the first plurality of control wells, a corresponding control vector comprising a plurality of elements, each respective element in the plurality of elements of the corresponding control vector including a measurement of a corresponding feature, in a plurality of features, of the aliquot of cells of the first cell type in the respective control well, thereby obtaining a first plurality of control vectors. The one or more programs further include instructions for obtaining, for each respective data well in the plurality of data wells, a corresponding data vector comprising the plurality of elements, each respective element in the plurality of elements of the corresponding data vector including a measurement of a corresponding feature, in the plurality of features, of the aliquot of cells of the first cell type in the respective data well, thereby obtaining a plurality of data vectors. The one or more programs further include instructions for forming a variability model based, at least in part, on all or a portion of a variance across the first plurality of control vectors. The one or more programs further include instructions for embedding each data vector in the plurality of data vectors by applying the variability model, thereby obtaining a set of variability model values for each data vector in the plurality of data vectors. The one or more programs further include instructions for using the set of variability model values and the corresponding data perturbation label of each data well in the plurality of data wells to resolve an effect of at least one data perturbation in the plurality of data perturbations on the first cell type.

In some aspects of the computer system, the first plurality of control wells is in a first subset of the plurality of plates, the plurality of data wells is in a second subset of the plurality of plates, and the second subset of the plurality of plates is other than the first subset of the plurality of plates.

In some aspects of the computer system, the first plurality of control wells consists of between 200 control wells and 1500 control wells in the second subset of the plurality of plates. In some aspects of this computer system, each control perturbation in the first plurality of control perturbations is a different siRNA.

In some aspects of the above described computer system the screen definition further includes a second plurality of control wells, there is an aliquot of cells of the first cell type in each control well in the second plurality of wells, the second plurality of control wells is present in each plate in the plurality of plates, and each respective control well in the second plurality of control wells is labeled with a control perturbation label corresponding to a control perturbation in a second plurality of control perturbations that is independently included in the respective control well and the second plurality of control wells collectively represents each control perturbation in the second plurality of control perturbations, the one or more programs further including instructions that: for each respective plate in the plurality of plates: obtain, for each respective control well in the second plurality of control wells of the respective plate, a corresponding normalization vector comprising the plurality of elements, each respective element in the plurality of elements of the normalization vector including a measurement of a corresponding feature, in the plurality of features, of the aliquot of cells of the first cell type in the respective control well, thereby obtaining a plurality of normalization vectors, and use the plurality of normalization vectors to normalize a set of data wells in the plurality of data wells that are in the respective plate prior to the obtaining. In some aspects of the computer system, the using the plurality of normalization vectors to normalize the set of data wells in the plurality of data wells that are in the respective plate comprises: computing a first measure of central tendency for each respective feature in the plurality of features across each corresponding normalization vector in the plurality of normalization features thereby forming a first plurality of measures of central tendency, each first measure of central tendency in the first plurality of measures of central tendency for a feature in the plurality of features; for each respective data well in the set of data wells in the plurality of data wells that are in the respective plate; for each respective feature in the plurality of features, subtracting a measured value for the respective feature by the first measure of central tendency corresponding to the respective feature and dividing the measured value for the respective feature by a standard deviation in measurement of the respective feature across the plurality of normalization vectors. In some aspects of the this computer system, the variability model is a plurality of dimension reduction components, and wherein the one or more programs further include instructions that: for each respective plate in the plurality of plates: obtain, for each respective control well in the second plurality of control wells of the respective plate, a corresponding dimension reduction normalization vector comprising a dimension reduction component value for each respective dimension reduction component, in the plurality of dimension reduction components by projecting the measurement of the corresponding features, in the plurality of features for the respective plate, specified by the respective dimension reduction component onto the respective dimension reduction component thereby obtaining a plurality of dimension reduction normalization vectors, and use the plurality of dimension reduction normalization vectors to standardize the set of data wells in the plurality of data wells that are in the respective plate prior to the computer. In some aspects this computer system, the using the plurality of dimension reduction normalization vectors to standardize the set of data wells in the plurality of data wells that are in the respective plate comprises: computing a second measure of central tendency for each respective dimension reduction component in the plurality of dimension reduction components across each corresponding dimension reduction normalization vector in the plurality of dimension reduction normalization vectors thereby forming a plurality of second measures of central tendency, each second measure of central tendency in the plurality of second measures of central tendency for a dimension reduction component in the plurality of dimension reduction components; for each respective data well in the set of data wells in the respective plate; for each respective dimension reduction component in the plurality of dimension reduction components, subtracting a measured value for the respective dimension reduction component by the second measure of central tendency corresponding to the respective dimension reduction component across the plurality of dimension reduction normalization vectors. In some aspects, for each respective control well in the second plurality of control wells, the plurality of elements of the corresponding normalization vector further comprises, for each respective feature in the plurality of features, a transform, selected from among a set of transforms in accordance with a feature transform lookup table, of the measurement of the respective feature in the respective control well. In some aspects, a transform in the set of transforms is a natural log transform of the measurement of the respective feature or a natural log transform of the measurement of the respective feature adjusted by a fixed increment.

In some aspects of the computer system described above, the instructions further comprise, prior to the forming, pruning the plurality of features by removing from the plurality of features each feature in the plurality of features that fails to satisfy a complexity threshold across the first plurality of control vectors.

In some aspects of the computer system described above, the variability model is a plurality of dimension reduction components, and wherein the plurality of dimension reduction components account for at least ninety percent of the variance of the plurality of features across the first plurality of control vectors. In some aspects, the plurality of dimension reduction components is a plurality of principal components and wherein the forming comprises applying principal component analysis to the plurality of features across the first plurality of control vectors.

In some aspects of the computer system described above, the variability model is a plurality of dimension reduction components, and wherein the plurality of dimension reduction components account for at least ninety-nine percent of the variance of the plurality of features across the first plurality of control vectors. In some aspects, the plurality of dimension reduction components is a plurality of principal components and wherein the forming comprises applying principal component analysis to the plurality of features across the first plurality of control vectors.

In some aspects of the computer system described above, for each respective control well in the first plurality of control wells, the plurality of elements of the corresponding control vector further comprises, for each respective feature in the plurality of features, a transform, selected from among a set of transforms in accordance with a feature transform lookup table, of the measurement of the respective feature in the respective control well, and for each respective data well in the plurality of data wells, the plurality of elements of the corresponding data vector further comprises, for each respective feature in the plurality of features, a transform, selected from among a set of transforms in accordance with the feature transform lookup table, of the measurement of the respective feature in the respective data well. In some aspects, a transform in the set of transforms is a natural log transform of the measurement of the respective feature or a natural log transform of the measurement of the respective feature adjusted by a fixed.

In some aspects, for each respective control well in the second plurality of control wells, the plurality of elements of the corresponding normalization vector further comprises, for each respective feature in the plurality of features, a transform, selected from among a set of transforms in accordance with a feature transform lookup table, of the measurement of the respective feature in the respective control well. In some aspects, a transform in the set of transforms is a natural log transform of the measurement of the respective feature or a natural log transform of the measurement of the respective feature adjusted by a fixed increment.

In some aspects, a transform in the set of transforms is a natural log transform of the measurement of the respective feature or a natural log transform of the measurement of the respective feature adjusted by a fixed increment. In some aspects, the set of transforms comprises (i) a natural log transform of the measurement of the respective feature, (ii) a natural log transform of the measurement of the respective feature adjusted by a first fixed increment, and (iii) a natural log transform of the measurement of the respective feature adjusted by a second fixed increment. In some aspects, the first fixed increment is 0.1 and the second fixed increment is 1.

In some aspects of the computer system described above, the first measure of central tendency for a respective feature is an arithmetic mean, weighted mean, midrange, midhinge, trimean, Winsorized mean, median, or mode of the respective feature across the plurality of normalization vectors.

In some aspects of the computer system described above the second measure of central tendency for a respective dimension reduction component is an arithmetic mean, weighted mean, midrange, midhinge, trimean, Winsorized mean, median, or mode of the respective dimension reduction component across the plurality of dimension reduction components.

In some aspects of the computer system described above, wherein each feature in the plurality of features represents a color, texture, or size of the cell or an enumerated portion of the cell.

In some aspects of the computer system described above, the obtaining a screen definition for a screen comprises imaging a corresponding well in the plurality of data wells or in the plurality of control wells to form a corresponding two-dimensional pixelated image having a corresponding plurality of native pixel values and wherein a different feature in the plurality of features arises as a result of a convolution or a series convolutions and pooling operators run against native pixel values in the corresponding plurality of native pixel values of the corresponding two-dimensional pixelated image.

In some aspects of the computer system described above, the aliquot of the cells of a respective control well is exposed to the respective control perturbation in the respective control well for at least one hour prior to obtaining the measurement of each feature in the plurality of features.

In some aspects of the computer system described above, the aliquot of the cells of a respective control well is exposed to the respective control perturbation in the respective control well for at least one hour, two hours, three hours, one day, two days, three days, four days, or five days prior to obtaining the measurement of each feature in the plurality of features.

In some aspects of the computer system described above, the aliquot of the cells of a respective data well is exposed to a data perturbation, in a plurality of data perturbations, in the respective data well for at least one hour prior to obtaining the measurement of each feature in the plurality of features. In some aspects, each data perturbation in the plurality of data perturbations is a different siRNA.

In some aspects of the computer system described above, each data perturbation in the plurality of data perturbations is a different siRNA., wherein the aliquot of the cells of a respective data well is exposed to a data perturbation, in a plurality of data perturbations, in the respective data well for at least one hour, two hours, three hours, one day, two days, three days, four days, or five days prior to obtaining the measurement of each feature in the plurality of features. In some aspects, each data perturbation in the plurality of data perturbations is a different siRNA.

In some aspects of the computer system described above, the variability model is a plurality of dimension reduction components that consists of between 100 dimension reduction components and 300 dimension reduction components.

In some aspects of the computer system described above, the variability model is a neural network.

In some aspects of the computer system described above, each feature in the plurality of features is an optical feature that is optically measured.

In some aspects of the computer system described above, a first subset of the plurality of features are optical features that are optically measured and a second subset of the plurality of features are non-optical features.

In some aspects of the computer system described above, each feature in the plurality of features is a feature that is non-optically measured.

In some aspects of the computer system described above, the plurality of control perturbations comprises a toxin, a cytokine, a predetermined drug, a siRNA, an sgRNA, a cell culture condition, or a genetic modification.

In some aspects of the computer system described above, each data perturbation in the plurality of data perturbations is a toxin, a cytokine, a predetermined drug, a siRNA, an sgRNA, a cell culture condition, or a genetic modification.

In one aspect of a method for evaluating an effect of one or more perturbations on cells of a first cell type, the method comprises: obtaining a screen definition for a screen, wherein the screen comprises a cell-based assay that is run on a temporarily contiguous basis using a plurality of multi-well plates, the screen definition identifies a first plurality of control wells and a plurality of data wells in the plurality of multi-well plates, each respective control well in the first plurality of control wells is labeled with a control perturbation label corresponding to a control perturbation in a first plurality of control perturbations that is independently included in the respective control well, each respective data well in the plurality of data wells is labeled with a data perturbation label corresponding to a data perturbation in a plurality of data perturbations that is independently included in the respective data well, and an aliquot of cells of the first cell type is included in each control well in the first plurality of control wells and in each data well in the plurality of data wells; obtaining, for each respective control well in the first plurality of control wells, a corresponding control vector comprising a plurality of elements, each respective element in the plurality of elements of the corresponding control vector including a measurement of a corresponding feature, in a plurality of features, of the aliquot of cells of the first cell type in the respective control well, thereby obtaining a first plurality of control vectors; obtaining, for each respective data well in the plurality of data wells, a corresponding data vector comprising the plurality of elements, each respective element in the plurality of elements of the corresponding data vector including a measurement of a corresponding feature, in the plurality of features, of the aliquot of cells of the first cell type in the respective data well, thereby obtaining a plurality of data vectors; forming a variability model based, at least in part, on all or a portion of a variance across the first plurality of control vectors; embedding each data vector in the plurality of data vectors onto the variability model, thereby obtaining a set of variability model values for each data vector in the plurality of data vectors; and using the set of variability model values and the corresponding data perturbation label of each data well in the plurality of data wells to resolve an effect of at least one data perturbation in the plurality of data perturbations on the first cell type.

With reference to the succinct computer system aspects described above, the various aspects of the method may be described in more detail in a similar manner to like aspects of the computer system.

In one aspect a non-transitory computer readable storage medium includes one or more computer programs embedded therein for evaluating an effect of one or more perturbations on cells of a first cell type. The one or more computer programs comprise instructions which, when executed by a computer system, cause the computer system to perform a method comprising: obtaining a screen definition for a screen, wherein the screen comprises a cell-based assay that is run on a temporarily contiguous basis using a plurality of multi-well plates, the screen definition identifies a first plurality of control wells and a plurality of data wells in the plurality of multi-well plates, each respective control well in the first plurality of control wells is labeled with a control perturbation label corresponding to a control perturbation in a first plurality of control perturbations that is independently included in the respective control well, each respective data well in the plurality of data wells is labeled with a data perturbation label corresponding to a data perturbation in a plurality of data perturbations that is independently included in the respective data well, and an aliquot of cells of the first cell type is included in each control well in the first plurality of control wells and in each data well in the plurality of data wells; obtaining, for each respective control well in the first plurality of control wells, a corresponding control vector comprising a plurality of elements, each respective element in the plurality of elements of the corresponding control vector including a measurement of a corresponding feature, in a plurality of features, of the aliquot of cells of the first cell type in the respective control well, thereby obtaining a first plurality of control vectors; obtaining, for each respective data well in the plurality of data wells, a corresponding data vector comprising the plurality of elements, each respective element in the plurality of elements of the corresponding data vector including a measurement of a corresponding feature, in the plurality of features, of the aliquot of cells of the first cell type in the respective data well, thereby obtaining a plurality of data vectors; forming a variability model based, at least in part, on all or a portion of a variance across the first plurality of control vectors; embedding each data vector in the plurality of data vectors onto the variability model, thereby obtaining a set of variability model values for each data vector in the plurality of data vectors; and using the set of variability model values and the corresponding data perturbation label of each data well in the plurality of data wells to resolve an effect of at least one data perturbation in the plurality of data perturbations on the first cell type.

With reference to the succinct computer system aspects described above, the various aspects of the non-transitory computer readable medium may be described in more detail in a similar manner to like aspects of the computer system.

### CONCLUSION

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. For instance, the computer program product could contain the program modules shown and/or described in any combination of FIGS. 1-7. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A computer system for evaluating an effect of one or more perturbations on cells of a first cell type, the computer system comprising:
one or more processors;
a memory; and
one or more programs, wherein the one or more programs are stored in the memory and are configured to be executed by the one or more processors, the one or more programs including instructions for:
obtaining a screen definition for a screen, wherein:
the screen comprises a cell-based assay that is run on a temporarily contiguous basis using a plurality of multi-well plates,
the screen definition identifies a first plurality of control wells and a plurality of data wells in the plurality of multi-well plates,
each respective control well in the first plurality of control wells is labeled with a control perturbation label corresponding to a control perturbation in a first plurality of control perturbations that is independently included in the respective control well,
each respective data well in the plurality of data wells is labeled with a data perturbation label corresponding to a data perturbation in a plurality of data perturbations that is independently included in the respective data well, and
an aliquot of cells of the first cell type is included in each control well in the first plurality of control wells and in each data well in the plurality of data wells;
obtaining, for each respective control well in the first plurality of control wells, a corresponding control vector comprising a plurality of elements, each respective element in the plurality of elements of the corresponding control vector including a measurement of a corresponding feature, in a plurality of features, of the aliquot of cells of the first cell type in the respective control well, thereby obtaining a first plurality of control vectors;
obtaining, for each respective data well in the plurality of data wells, a corresponding data vector comprising the plurality of elements, each respective element in the plurality of elements of the corresponding data vector including a measurement of a corresponding feature, in the plurality of features, of the aliquot of cells of the first cell type in the respective data well, thereby obtaining a plurality of data vectors;
forming a variability model based, at least in part, on all or a portion of a variance across the first plurality of control vectors;
embedding each data vector in the plurality of data vectors by applying the variability model, thereby obtaining a set of variability model values for each data vector in the plurality of data vectors; and
using the set of variability model values and the corresponding data perturbation label of each data well in the plurality of data wells to resolve an effect of at least one data perturbation in the plurality of data perturbations on the first cell type.

2. The computer system of claim 1, wherein:
the first plurality of control wells is in a first subset of the plurality of multi-well plates,
the plurality of data wells is in a second subset of the plurality of multi-well plates, and
the second subset of the plurality of multi-well plates is other than the first subset of the plurality of multi-well plates.

3. The computer system of claim 2, wherein the first plurality of control wells consists of between 200 control wells and 1500 control wells in the second subset of the plurality of multi-well plates.

4. The computer system of claim 3, wherein each control perturbation in the first plurality of control perturbations is a different siRNA.

5. The computer system of claim 1, wherein
the screen definition further includes a second plurality of control wells,
there is an aliquot of cells of the first cell type in each control well in the second plurality of control wells,
the second plurality of control wells is present in each multi-well plate in the plurality of multi-well plates,
each respective control well in the second plurality of control wells is labeled with a control perturbation label corresponding to a control perturbation in a second plurality of control perturbations that is independently included in the respective control well and the second plurality of control wells collectively represents each control perturbation in the second plurality of control perturbations, and
the one or more programs further including instructions that:
for each respective multi-well plate in the plurality of multi-well plates:
obtain, for each respective control well in the second plurality of control wells of the respective multi-well plate, a corresponding normalization vector comprising the plurality of elements, each respective element in the plurality of elements of the corresponding normalization vector including a measurement of a corresponding feature, in the plurality of features, of the aliquot of cells of the first cell type in the respective control well, thereby obtaining a plurality of normalization vectors, and
use the plurality of normalization vectors to normalize a set of data wells in the plurality of data wells that are in the respective multi-well plate prior to the obtaining the screen definition for the screen.

6. The computer system of claim 5, wherein
the using the plurality of normalization vectors to normalize the set of data wells in the plurality of data wells that are in the respective multi-well plate comprises:
computing a first measure of central tendency for each respective feature in the plurality of features across each corresponding normalization vector in the plurality of normalization vectors thereby forming a first plurality of measures of central tendency, each first measure of central tendency in the first plurality of measures of central tendency for a feature in the plurality of features; and
for each respective data well in the set of data wells in the plurality of data wells that are in the respective multi-well plate;
for each respective feature in the plurality of features, subtracting a measured value for the respective feature by the first measure of central tendency corresponding to the respective feature and dividing the measured value for the respective feature by a standard deviation in measurement of the respective feature across the plurality of normalization vectors.

7. The computer system of claim 6, wherein the variability model is a plurality of dimension reduction components, and wherein the one or more programs further include instructions that:
for each respective multi-well plate in the plurality of multi-well plates:
obtain, for each respective control well in the second plurality of control wells of the respective multi-well plate, a corresponding dimension reduction normalization vector comprising a dimension reduction component value for each respective dimension reduction component, in the plurality of dimension reduction components by projecting the measurement of the corresponding features, in the plurality of features for the respective multi-well plate, specified by the respective dimension reduction component onto the respective dimension reduction component thereby obtaining a plurality of dimension reduction normalization vectors, and
use the plurality of dimension reduction normalization vectors to standardize the set of data wells in the plurality of data wells that are in the respective multi-well plate prior to the computing.

8. The computer system of claim 7, wherein
the using the plurality of dimension reduction normalization vectors to standardize the set of data wells in the plurality of data wells that are in the respective multi-well plate comprises:
computing a second measure of central tendency for each respective dimension reduction component in the plurality of dimension reduction components across each corresponding dimension reduction normalization vector in the plurality of dimension reduction normalization vectors thereby forming a plurality of second measures of central tendency, each second measure of central tendency in the plurality of second measures of central tendency for a dimension reduction component in the plurality of dimension reduction components; and
for each respective data well in the set of data wells in the respective multi-well plate;
for each respective dimension reduction component in the plurality of dimension reduction components, subtracting a measured value for the respective dimension reduction component by the second measure of central tendency corresponding to the respective dimension reduction component across the plurality of dimension reduction normalization vectors.

9. The computer system of claim 1, wherein the instructions further comprise, prior to the forming, pruning the plurality of features by removing from the plurality of features each feature in the plurality of features that fails to satisfy a complexity threshold across the first plurality of control vectors.

10. The computer system of claim 1, wherein the variability model is a plurality of dimension reduction components, and wherein the plurality of dimension reduction components account for at least ninety percent of the variance of the plurality of features across the first plurality of control vectors.

11. The computer system of claim 1, wherein the variability model is a plurality of dimension reduction components, and wherein the plurality of dimension reduction components account for at least ninety-nine percent of the variance of the plurality of features across the first plurality of control vectors.

12. The computer system of claim 11, wherein the plurality of dimension reduction components is a plurality of principal components and wherein the forming comprises applying principal component analysis to the plurality of features across the first plurality of control vectors.

13. The computer system of claim 1, wherein,
for each respective control well in the first plurality of control wells, the plurality of elements of the corresponding control vector further comprises, for each respective feature in the plurality of features, a transform, selected from among a set of transforms in accordance with a feature transform lookup table, of the measurement of the respective feature in the respective control well, and
for each respective data well in the plurality of data wells, the plurality of elements of the corresponding data vector further comprises, for each respective feature in the plurality of features, a transform, selected from among a set of transforms in accordance with the feature transform lookup table, of the measurement of the respective feature in the respective data well.

14. The computer system of claim 1, wherein the variability model is a neural network.

15. The computer system of claim 1, wherein each feature in the plurality of features is an optical feature that is optically measured.

16. A method for evaluating an effect of one or more perturbations on cells of a first cell type, the method comprising:
obtaining a screen definition for a screen, wherein
the screen comprises a cell-based assay that is run on a temporarily contiguous basis using a plurality of multi-well plates,
the screen definition identifies a first plurality of control wells and a plurality of data wells in the plurality of multi-well plates,
each respective control well in the first plurality of control wells is labeled with a control perturbation label corresponding to a control perturbation in a first plurality of control perturbations that is independently included in the respective control well,
each respective data well in the plurality of data wells is labeled with a data perturbation label corresponding to a data perturbation in a plurality of data perturbations that is independently included in the respective data well, and
an aliquot of cells of the first cell type is included in each control well in the first plurality of control wells and in each data well in the plurality of data wells;
obtaining, for each respective control well in the first plurality of control wells, a corresponding control vector comprising a plurality of elements, each respective element in the plurality of elements of the corresponding control vector including a measurement of a corresponding feature, in a plurality of features, of the aliquot of cells of the first cell type in the respective control well, thereby obtaining a first plurality of control vectors;
obtaining, for each respective data well in the plurality of data wells, a corresponding data vector comprising the plurality of elements, each respective element in the plurality of elements of the corresponding data vector including a measurement of a corresponding feature, in the plurality of features, of the aliquot of cells of the first cell type in the respective data well, thereby obtaining a plurality of data vectors;
forming a variability model based, at least in part, on all or a portion of a variance across the first plurality of control vectors;
embedding each data vector in the plurality of data vectors onto the variability model, thereby obtaining a set of variability model values for each data vector in the plurality of data vectors; and
using the set of variability model values and the corresponding data perturbation label of each data well in the plurality of data wells to resolve an effect of at least one data perturbation in the plurality of data perturbations on the first cell type.

17. The method of claim 16, wherein the method further comprises:
prior to the forming the variability model, pruning the plurality of features by removing from the plurality of features each feature in the plurality of features that fails to satisfy a complexity threshold across the first plurality of control vectors.

18. The method of claim 16, wherein the obtaining a screen definition for a screen comprises:
imaging a corresponding well in the plurality of data wells or in the first plurality of control wells to form a corresponding two-dimensional pixelated image having a corresponding plurality of native pixel values and wherein a different feature in the plurality of features arises as a result of a convolution or a series convolutions and pooling operators run against native pixel values in the corresponding plurality of native pixel values of the corresponding two-dimensional pixelated image.

19. The method of claim 16, wherein the variability model is a plurality of dimension reduction components, and the plurality of dimension reduction components is a plurality of principal components and wherein the forming a variability model comprises:
applying principal component analysis to the plurality of features across the first plurality of control vectors.

20. A non-transitory computer readable storage medium and one or more computer programs embedded therein for evaluating an effect of one or more perturbations on cells of a first cell type, the one or more computer programs comprising instructions which, when executed by a computer system, cause the computer system to perform a method comprising:
obtaining a screen definition for a screen, wherein
the screen comprises a cell-based assay that is run on a temporarily contiguous basis using a plurality of multi-well plates,
the screen definition identifies a first plurality of control wells and a plurality of data wells in the plurality of multi-well plates,
each respective control well in the first plurality of control wells is labeled with a control perturbation label corresponding to a control perturbation in a first plurality of control perturbations that is independently included in the respective control well,
each respective data well in the plurality of data wells is labeled with a data perturbation label corresponding to a data perturbation in a plurality of data perturbations that is independently included in the respective data well, and
an aliquot of cells of the first cell type is included in each control well in the first plurality of control wells and in each data well in the plurality of data wells;
obtaining, for each respective control well in the first plurality of control wells, a corresponding control vector comprising a plurality of elements, each respective element in the plurality of elements of the corresponding control vector including a measurement of a corresponding feature, in a plurality of features, of the aliquot of cells of the first cell type in the respective control well, thereby obtaining a first plurality of control vectors;
obtaining, for each respective data well in the plurality of data wells, a corresponding data vector comprising the plurality of elements, each respective element in the plurality of elements of the corresponding data vector including a measurement of a corresponding feature, in the plurality of features, of the aliquot of cells of the first cell type in the respective data well, thereby obtaining a plurality of data vectors;
forming a variability model based, at least in part, on all or a portion of a variance across the first plurality of control vectors;
embedding each data vector in the plurality of data vectors onto the variability model, thereby obtaining a set of variability model values for each data vector in the plurality of data vectors; and
using the set of variability model values and the corresponding data perturbation label of each data well in the plurality of data wells to resolve an effect of at least one data perturbation in the plurality of data perturbations on the first cell type.
